SEQUENCE LISTING (272 sequences)

SEQ ID NO: 1 - protein, 18 aa, Ratus Norvegicus
AADKDNCFATEGPNLVAR

SEQ ID NO: 2 - protein, 11 aa, Ratus Norvegicus
ADAGGELDLAR

SEQ ID NO: 3 - protein, 9 aa, Ratus Norvegicus
AEVQTLVSR

SEQ ID NO: 4 - protein, 9 aa, Ratus Norvegicus
AFEEEQALR

SEQ ID NO: 5 - protein, 13 aa, Ratus Norvegicus
AFPAWADTSILSR

SEQ ID NO: 6 - protein, 10 aa, Ratus Norvegicus
AGFAGDDAPR

SEQ ID NO: 7 - protein, 10 aa, Ratus Norvegicus
AIDVGQGQTR

SEQ ID NO: 8 - protein, 12 aa, Ratus Norvegicus
ALEESNYELEGK

SEQ ID NO: 9 - protein, 10 aa, Ratus Norvegicus
ALGLSNFSSR

SEQ ID NO: 10 - protein, 13 aa, Ratus Norvegicus
ANVDKPGLVDDFK

SEQ ID NO: 11 - protein, 10 aa, Ratus Norvegicus
APDFVFYAPR

SEQ ID NO: 12 - protein, 14 aa, Ratus Norvegicus
APQVSTPTLVEAAR

SEQ ID NO: 13 - protein, 14 aa, Ratus Norvegicus
APQVSTPTLVEAAR

SEQ ID NO: 14 - protein, 14 aa, Ratus Norvegicus
ARPFPDGLAEDIDK

SEQ ID NO: 15 - protein, 15 aa, Ratus Norvegicus
ASAELALGENSEVLK

SEQ ID NO: 16 - protein, 13 aa, Ratus Norvegicus
ASSTANLIFEDCR

SEQ ID NO: 17 - protein, 12 aa, Ratus Norvegicus
ASSVVVSGTPIR

SEQ ID NO: 18 - protein, 11 aa, Ratus Norvegicus
ATDFVVPGPGK

SEQ ID NO: 19 - protein, 10 aa, Ratus Norvegicus
AVAFQNPQTR

SEQ ID NO: 20 - protein, 10 aa, Ratus Norvegicus
AVDSLVPIGR

SEQ ID NO: 21 - protein, 9 aa, Ratus Norvegicus
AVFPSIVGR

SEQ ID NO: 22 - protein, 15 aa, Ratus Norvegicus
AVLVDLEPGTMDSVR

SEQ ID NO: 23 - protein, 12 aa, Ratus Norvegicus
CAVVDVPFGGAK

SEQ ID NO: 24 - protein, 9 aa, Ratus Norvegicus
CCSGSLVER

SEQ ID NO: 25 - protein, 9 aa, Ratus Norvegicus
CCTLPEAQR

SEQ ID NO: 26 - protein, 15 aa, Ratus Norvegicus
CNADPGLSALLSDHR

SEQ ID NO: 27 - protein, 12 aa, Ratus Norvegicus
CNVSEGVAQCTR

SEQ ID NO: 28 - protein, 13 aa, Ratus Norvegicus
DAGHPLYPFNDPY

SEQ ID NO: 29 - protein, 10 aa, Ratus Norvegicus
DAGMQLQGYR

SEQ ID NO: 30 - protein, 12 aa, Ratus Norvegicus
DAGTIAGLNVLR

SEQ ID NO: 31 - protein, 8 aa, Ratus Norvegicus
DAQLFIQR

SEQ ID NO: 32 - protein, 12 aa, Ratus Norvegicus
DDGSWEVIEGYR

SEQ ID NO: 33 - protein, 13 aa, Ratus Norvegicus
DDNGKPYVLPSVR

SEQ ID NO: 34 - protein, 11 aa, Ratus Norvegicus
DDNPNLPPFQR

SEQ ID NO: 35 - protein, 12 aa, Ratus Norvegicus
DFDPAINEYIQR

SEQ ID NO: 36 - protein, 13 aa, Ratus Norvegicus
DLDVAVLVGSMPR

SEQ ID NO: 37 - protein, 14 aa, Ratus Norvegicus
DLGATWVVLGHSER

SEQ ID NO: 38 - protein, 8 aa, Ratus Norvegicus
DMDLYSYR

SEQ ID NO: 39 - protein, 8 aa, Ratus Norvegicus
DNIIDLTK

SEQ ID NO: 40 - protein, 12 aa, Ratus Norvegicus
DNYGELADCCAK

SEQ ID NO: 41 - protein, 8 aa, Ratus Norvegicus
DPQHDLER

SEQ ID NO: 42 - protein, 10 aa, Ratus Norvegicus
DSTLIMQLLR

SEQ ID NO: 43 - protein, 9 aa, Ratus Norvegicus
DTPGFIVNR

SEQ ID NO: 44 - protein, 9 aa, Ratus Norvegicus
DYFISCPGR

SEQ ID NO: 45 - protein, 10 aa, Ratus Norvegicus
EAFNMIDQNR

SEQ ID NO: 46 - protein, 14 aa, Ratus Norvegicus
ECCHGDLLECADDR

SEQ ID NO: 47 - protein, 10 aa, Ratus Norvegicus
EDGGGWWYNR

SEQ ID NO: 48 - protein, 9 aa, Ratus Norvegicus
EESLALAVK

SEQ ID NO: 49 - protein, 10 aa, Ratus Norvegicus
EGASILLDGR

SEQ ID NO: 50 - protein, 10 aa, Ratus Norvegicus
EHLFPTSQVK

SEQ ID NO: 51 - protein, 8 aa, Ratus Norvegicus
EIMIAAQR

SEQ ID NO: 52 - protein, 10 aa, Ratus Norvegicus
EKIEENGSMR

SEQ ID NO: 53 - protein, 8 aa, Ratus Norvegicus
EKIEMELR

SEQ ID NO: 54 - protein, 8 aa, Ratus Norvegicus
ELADIAHR

SEQ ID NO: 55 - protein, 12 aa, Ratus Norvegicus
ELFAQEAFAPFR

SEQ ID NO: 56 - protein, 8 aa, Ratus Norvegicus
ELYLVAYK

SEQ ID NO: 57 - protein, 8 aa, Ratus Norvegicus
ENFSCLTR

SEQ ID NO: 58 - protein, 11 aa, Ratus Norvegicus
ENMAYTVEGIR

SEQ ID NO: 59 - protein, 9 aa, Ratus Norvegicus
EPPFPLSTR

SEQ ID NO: 60 - protein, 10 aa, Ratus Norvegicus
EQIDIFEGIK

SEQ ID NO: 61 - protein, 10 aa, Ratus Norvegicus
ETYLAILMDR

SEQ ID NO: 62 - protein, 10 aa, Ratus Norvegicus
EVAEQFLNIR

SEQ ID NO: 63 - protein, 8 aa, Ratus Norvegicus
EVAGFWVK

SEQ ID NO: 64 - protein, 15 aa, Ratus Norvegicus
EVGVYEALKDDSWLK

SEQ ID NO: 65 - protein, 12 aa, Ratus Norvegicus
EVLDILTAELHR

SEQ ID NO: 66 - protein, 10 aa, Ratus Norvegicus
FAELAQIYAR

SEQ ID NO: 67 - protein, 11 aa, Ratus Norvegicus
FANTMGLVIER

SEQ ID NO: 68 - protein, 10 aa, Ratus Norvegicus
FEELNADLFR

SEQ ID NO: 69 - protein, 11 aa, Ratus Norvegicus
FELTGIPPAPR

SEQ ID NO: 70 - protein, 9 aa, Ratus Norvegicus
FGEPIPISK

SEQ ID NO: 71 - protein, 10 aa, Ratus Norvegicus
FIQSPEDLEK

SEQ ID NO: 72 - protein, 12 aa, Ratus Norvegicus
FLIPNASQPESK

SEQ ID NO: 73 - protein, 11 aa, Ratus Norvegicus
FNPVTGEVPPR

SEQ ID NO: 74 - protein, 13 aa, Ratus Norvegicus
FNSANEDNVTQVR

SEQ ID NO: 75 - protein, 11 aa, Ratus Norvegicus
FNVWDTAGQEK

SEQ ID NO: 76 - protein, 10 aa, Ratus Norvegicus
FPGQLNADLR

SEQ ID NO: 77 - protein, 11 aa, Ratus Norvegicus
FPNAEFAEITK

SEQ ID NO: 78 - protein, 12 aa, Ratus Norvegicus
FPPDNSAPYGAR

SEQ ID NO: 79 - protein, 15 aa, Ratus Norvegicus
FSTVAGESGSADTVR

SEQ ID NO: 80 - protein, 15 aa, Ratus Norvegicus
FTPGTFTNQIQAAFR

SEQ ID NO: 81 - protein, 12 aa, Ratus Norvegicus
FVEGLPINDFSR

SEQ ID NO: 82 - protein, 14 aa, Ratus Norvegicus
FVTVQTISGTGALR

SEQ ID NO: 83 - protein, 10 aa, Ratus Norvegicus
GAEIVADTFR

SEQ ID NO: 84 - protein, 12 aa, Ratus Norvegicus
GATQQILDEAER

SEQ ID NO: 85 - protein, 13 aa, Ratus Norvegicus
GAVHQLCQSLAGK

SEQ ID NO: 86 - protein, 9 aa, Ratus Norvegicus
GDFCIQVGR

SEQ ID NO: 87 - protein, 15 aa, Ratus Norvegicus
GECQSEGVLFFQGNR

SEQ ID NO: 88 - protein, 10 aa, Ratus Norvegicus
GEFITTVQQR

SEQ ID NO: 89 - protein, 10 aa, Ratus Norvegicus
GGGQIIPTAR

SEQ ID NO: 90 - protein, 13 aa, Ratus Norvegicus
GIMGEDSYPYIGK

SEQ ID NO: 91 - protein, 11 aa, Ratus Norvegicus
GLAPEQPVTLR

SEQ ID NO: 92 - protein, 11 aa, Ratus Norvegicus
GLDPYNMLPPK

SEQ ID NO: 93 - protein, 15 aa, Ratus Norvegicus
GLEVTAYSPLGSSDR

SEQ ID NO: 94 - protein, 16 aa, Ratus Norvegicus
GLGTDEDSILNLLTAR

SEQ ID NO: 95 - protein, 20 aa, Ratus Norvegicus
GNDISSGTVLSEYVGSGPPK

SEQ ID NO: 96 - protein, 10 aa, Ratus Norvegicus
GNFNYIEFTR

SEQ ID NO: 97 - protein, 12 aa, Ratus Norvegicus
GNLTDLETNGVR

SEQ ID NO: 98 - protein, 12 aa, Ratus Norvegicus
GQIIQVEAPWIK

SEQ ID NO: 99 - protein, 12 aa, Ratus Norvegicus
GTVTDFSGFDGR

SEQ ID NO: 100 - protein, 10 aa, Ratus Norvegicus
GYSFTTTAER

SEQ ID NO: 101 - protein, 9 aa, Ratus Norvegicus
HAFGAPLTK

SEQ ID NO: 102 - protein, 8 aa, Ratus Norvegicus
HFMAPGVR

SEQ ID NO: 103 - protein, 12 aa, Ratus Norvegicus
HGGPFCAGDATR

SEQ ID NO: 104 - protein, 16 aa, Ratus Norvegicus
HGGTIPVVPTAEFQDR

SEQ ID NO: 105 - protein, 16 aa, Ratus Norvegicus
HGGTIPVVPTAEFQDR

SEQ ID NO: 106 - protein, 11 aa, Ratus Norvegicus
HHPEDVEPAVR

SEQ ID NO: 107 - protein, 10 aa, Ratus Norvegicus
HLFTGPVLSK

SEQ ID NO: 108 - protein, 8 aa, Ratus Norvegicus
HLTGEFEK

SEQ ID NO: 109 - protein, 11 aa, Ratus Norvegicus
HTTIFEVLPQK

SEQ ID NO: 110 - protein, 14 aa, Ratus Norvegicus
HVPGASFFDIEECR

SEQ ID NO: 111 - protein, 10 aa, Ratus Norvegicus
IEVYMDGGVR

SEQ ID NO: 112 - protein, 12 aa, Ratus Norvegicus
IEYFEEAVNYLR

SEQ ID NO: 113 - protein, 10 aa, Ratus Norvegicus
IGAEVYHNLK

SEQ ID NO: 114 - protein, 9 aa, Ratus Norvegicus
IGFPWSEIR

SEQ ID NO: 115 - protein, 11 aa, Ratus Norvegicus
IGGIGTVPVGR

SEQ ID NO: 116 - protein, 15 aa, Ratus Norvegicus
ILGADTSVDLEETGR

SEQ ID NO: 117 - protein, 10 aa, Ratus Norvegicus
INFDDNAEFR

SEQ ID NO: 118 - protein, 11 aa, Ratus Norvegicus
INISEGNCPER

SEQ ID NO: 119 - protein, 9 aa, Ratus Norvegicus
IPSHAVVAR

SEQ ID NO: 120 - protein, 10 aa, Ratus Norvegicus
IQLVEEELDR

SEQ ID NO: 121 - protein, 14 aa, Ratus Norvegicus
IQVLQQQADDAEER

SEQ ID NO: 122 - protein, 10 aa, Ratus Norvegicus
ISEQFTAMFR

SEQ ID NO: 123 - protein, 13 aa, Ratus Norvegicus
ITEIYEGTSEIQR

SEQ ID NO: 124 - protein, 11 aa, Ratus Norvegicus
IWHHTFYNELR

SEQ ID NO: 125 - protein, 13 aa, Ratus Norvegicus
KPPPDGHYVDVVR

SEQ ID NO: 126 - protein, 13 aa, Ratus Norvegicus
KPVDQYEDCYLAR

SEQ ID NO: 127 - protein, 8 aa, Ratus Norvegicus
KYEATLEK

SEQ ID NO: 128 - protein, 12 aa, Ratus Norvegicus
LADMALALESAR

SEQ ID NO: 129 - protein, 11 aa, Ratus Norvegicus
LAQEDPDYGLR

SEQ ID NO: 130 - protein, 10 aa, Ratus Norvegicus
LASDLLEWIR

SEQ ID NO: 131 - protein, 10 aa, Ratus Norvegicus
LAVNMVPFPR

SEQ ID NO: 132 - protein, 9 aa, Ratus Norvegicus
LCEAHGITR

SEQ ID NO: 133 - protein, 9 aa, Ratus Norvegicus
LCVAHGITR

SEQ ID NO: 134 - protein, 7 aa, Ratus Norvegicus
LCVLHEK

SEQ ID NO: 135 - protein, 14 aa, Ratus Norvegicus
LEGTNVQEAQNILK

SEQ ID NO: 136 - protein, 13 aa, Ratus Norvegicus
LFIVGSNSSSSTR

SEQ ID NO: 137 - protein, 9 aa, Ratus Norvegicus
LGVTADDVK

SEQ ID NO: 138 - protein, 14 aa, Ratus Norvegicus
LHFFMPGFAPLTSR

SEQ ID NO: 139 - protein, 14 aa, Ratus Norvegicus
LISWYDNEYGYSNR

SEQ ID NO: 140 - protein, 8 aa, Ratus Norvegicus
LITLEQGK

SEQ ID NO: 141 - protein, 8 aa, Ratus Norvegicus
LLVVTDPR

SEQ ID NO: 142 - protein, 14 aa, Ratus Norvegicus
LNGDWFSIVVASDK

SEQ ID NO: 143 - protein, 14 aa, Ratus Norvegicus
LNGDWFSIVVASNK

SEQ ID NO: 144 - protein, 9 aa, Ratus Norvegicus
LNIMTAGPR

SEQ ID NO: 145 - protein, 14 aa, Ratus Norvegicus
LPCVEDYLSAILNR

SEQ ID NO: 146 - protein, 8 aa, Ratus Norvegicus
LPLQDVYK

SEQ ID NO: 147 - protein, 10 aa, Ratus Norvegicus
LPSDVVTAVR

SEQ ID NO: 148 - protein, 11 aa, Ratus Norvegicus
LQHGSILGFPK

SEQ ID NO: 149 - protein, 13 aa, Ratus Norvegicus
LQSIGTENTEENR

SEQ ID NO: 150 - protein, 13 aa, Ratus Norvegicus
LTFDSSFSPNTGK

SEQ ID NO: 151 - protein, 8 aa, Ratus Norvegicus
LVAMKFLR

SEQ ID NO: 152 - protein, 10 aa, Ratus Norvegicus
LVIIEGDLER

SEQ ID NO: 153 - protein, 10 aa, Ratus Norvegicus
LVQEVTDFAK

SEQ ID NO: 154 - protein, 14 aa, Ratus Norvegicus
LYTLVLTDPDAPSR

SEQ ID NO: 155 - protein, 7 aa, Ratus Norvegicus
LYYFQGR

SEQ ID NO: 156 - protein, 12 aa, Ratus Norvegicus
MAENLGFLGSLK

SEQ ID NO: 157 - protein, 9 aa, Ratus Norvegicus
MGFEPLAYK

SEQ ID NO: 158 - protein, 13 aa, Ratus Norvegicus
MGLALISGYNLFR

SEQ ID NO: 159 - protein, 9 aa, Ratus Norvegicus
MNLGVGAYR

SEQ ID NO: 160 - protein, 10 aa, Ratus Norvegicus
MPINEPAPGR

SEQ ID NO: 161 - protein, 9 aa, Ratus Norvegicus
TEDIITTIR

SEQ ID NO: 162 - protein, 10 aa, Ratus Norvegicus
MPLIGLGTWK

SEQ ID NO: 163 - protein, 8 aa, Ratus Norvegicus
MVEGFFDR

SEQ ID NO: 164 - protein, 13 aa, Ratus Norvegicus
NALANPLYCPDYR

SEQ ID NO: 165 - protein, 13 aa, Ratus Norvegicus
NGETFQAMVLYGR

SEQ ID NO: 166 - protein, 13 aa, Ratus Norvegicus
NGETFQLMVLYGR

SEQ ID NO: 167 - protein, 13 aa, Ratus Norvegicus
NGQGSDPAVTYYR

SEQ ID NO: 168 - protein, 10 aa, Ratus Norvegicus
NHFTVAQNER

SEQ ID NO: 169 - protein, 8 aa, Ratus Norvegicus
NLLSVAYK

SEQ ID NO: 170 - protein, 9 aa, Ratus Norvegicus
NLPVEEAGR

SEQ ID NO: 171 - protein, 10 aa, Ratus Norvegicus
NLQYYDISAK

SEQ ID NO: 172 - protein, 9 aa, Ratus Norvegicus
NPDSLELIR

SEQ ID NO: 173 - protein, 9 aa, Ratus Norvegicus
NPSVLLTLR

SEQ ID NO: 174 - protein, 11 aa, Ratus Norvegicus
NPVTSVDAAFR

SEQ ID NO: 175 - protein, 15 aa, Ratus Norvegicus
NQVAMNPTNTVFDAK

SEQ ID NO: 176 - protein, 7 aa, Ratus Norvegicus
NVPNWHR

SEQ ID NO: 177 - protein, 15 aa, Ratus Norvegicus
NVQAEEMVEFSSGLK

SEQ ID NO: 178 - protein, 13 aa, Ratus Norvegicus
QEYDESGPSIVHR

SEQ ID NO: 179 - protein, 13 aa, Ratus Norvegicus
QFDIQLLTHNDPK

SEQ ID NO: 180 - protein, 10 aa, Ratus Norvegicus
QGEIFLLPAR

SEQ ID NO: 181 - protein, 12 aa, Ratus Norvegicus
QIDDVLSVASVR

SEQ ID NO: 182 - protein, 13 aa, Ratus Norvegicus
QLDEVSASIDALR

SEQ ID NO: 183 - protein, 13 aa, Ratus Norvegicus
QLLTLSNELSQAR

SEQ ID NO: 184 - protein, 9 aa, Ratus Norvegicus
QTALAELVK

SEQ ID NO: 185 - protein, 12 aa, Ratus Norvegicus
RFDDAVVQSDMK

SEQ ID NO: 186 - protein, 8 aa, Ratus Norvegicus
RPEFQALR

SEQ ID NO: 187 - protein, 8 aa, Ratus Norvegicus
RPEVDGVR

SEQ ID NO: 188 - protein, 16 aa, Ratus Norvegicus
RPNSGSLIQVVTTDGK

SEQ ID NO: 189 - protein, 8 aa, Ratus Norvegicus
RWEVAALR

SEQ ID NO: 190 - protein, 7 aa, Ratus Norvegicus
RYEEIVK

SEQ ID NO: 191 - protein, 9 aa, Ratus Norvegicus
SCAHDWVYE

SEQ ID NO: 192 - protein, 12 aa, Ratus Norvegicus
SCWDEPLSITVR

SEQ ID NO: 193 - protein, 12 aa, Ratus Norvegicus
SENEPIENEAAR

SEQ ID NO: 194 - protein, 11 aa, Ratus Norvegicus
SHGQDYLVGNR

SEQ ID NO: 195 - protein, 9 aa, Ratus Norvegicus
SIHTLFGDK

SEQ ID NO: 196 - protein, 10 aa, Ratus Norvegicus
SIQEIQELDK

SEQ ID NO: 197 - protein, 9 aa, Ratus Norvegicus
SLHTLFGDK

SEQ ID NO: 198 - protein, 14 aa, Ratus Norvegicus
SNYNFEKPFLWLAR

SEQ ID NO: 199 - protein, 8 aa, Ratus Norvegicus
SPAQILLR

SEQ ID NO: 200 - protein, 14 aa, Ratus Norvegicus
SQIHDIVLVGGSTR

SEQ ID NO: 201 - protein, 11 aa, Ratus Norvegicus
SRPSLPLPQSR

SEQ ID NO: 202 - protein, 10 aa, Ratus Norvegicus
SSEEIESAFR

SEQ ID NO: 203 - protein, 16 aa, Ratus Norvegicus
STAGDTHLGGEDFDNR

SEQ ID NO: 204 - protein, 11 aa, Ratus Norvegicus
SVSLQYLEAVR

SEQ ID NO: 205 - protein, 14 aa, Ratus Norvegicus
SVTEQGAELSNEER

SEQ ID NO: 206 - protein, 9 aa, Ratus Norvegicus
SWNETFHTR

SEQ ID NO: 207 - protein, 7 aa, Ratus Norvegicus
SWVEENR

SEQ ID NO: 208 - protein, 16 aa, Ratus Norvegicus
SYELPDGQVITIGNER

SEQ ID NO: 209 - protein, 9 aa, Ratus Norvegicus
SYLSWLTER

SEQ ID NO: 210 - protein, 13 aa, Ratus Norvegicus
TATPQQAQEVHEK

SEQ ID NO: 211 - protein, 9 aa, Ratus Norvegicus
TAVCDIPPR

SEQ ID NO: 212 - protein, 13 aa, Ratus Norvegicus
TCVADENAENCDK

SEQ ID NO: 213 - protein, 11 aa, Ratus Norvegicus
TDYMVGSYGPR

SEQ ID NO: 214 - protein, 8 aa, Ratus Norvegicus
TEDIITTI

SEQ ID NO: 215 - protein, 12 aa, Ratus Norvegicus
TENGGWTVIQNR

SEQ ID NO: 216 - protein, 14 aa, Ratus Norvegicus
TEQGPPSSEYIFER

SEQ ID NO: 217 - protein, 10 aa, Ratus Norvegicus
TFESLVDFCK

SEQ ID NO: 218 - protein, 16 aa, Ratus Norvegicus
TGAIVDVPVGDELLGR

SEQ ID NO: 219 - protein, 11 aa, Ratus Norvegicus
TGKPNPDQLLK

SEQ ID NO: 220 - protein, 13 aa, Ratus Norvegicus
TGTAEMSSILEER

SEQ ID NO: 221 - protein, 12 aa, Ratus Norvegicus
TINEVENQILTR

SEQ ID NO: 222 - protein, 11 aa, Ratus Norvegicus
TLADAEGDVFR

SEQ ID NO: 223 - protein, 15 aa, Ratus Norvegicus
TLNEWSSQISPDLVR

SEQ ID NO: 224 - protein, 8 aa, Ratus Norvegicus
TNCELYEK

SEQ ID NO: 225 - protein, 11 aa, Ratus Norvegicus
TPAQFDADELR

SEQ ID NO: 226 - protein, 11 aa, Ratus Norvegicus
TQAMGLWAQPR

SEQ ID NO: 227 - protein, 13 aa, Ratus Norvegicus
TTPSYVAFTDTER

SEQ ID NO: 228 - protein, 13 aa, Ratus Norvegicus
TVEAEAAHGTVTR

SEQ ID NO: 229 - protein, 9 aa, Ratus Norvegicus
TVLPADGPR

SEQ ID NO: 230 - protein, 10 aa, Ratus Norvegicus
TVSVLNGGFR

SEQ ID NO: 231 - protein, 18 aa, Ratus Norvegicus
VAPEEHPVLLTEAPLNPK

SEQ ID NO: 232 - protein, 9 aa, Ratus Norvegicus
VASFEEVVR

SEQ ID NO: 233 - protein, 13 aa, Ratus Norvegicus
VDYGGVTVDELGK

SEQ ID NO: 234 - protein, 11 aa, Ratus Norvegicus
VEIIANDQGNR

SEQ ID NO: 235 - protein, 10 aa, Ratus Norvegicus
VGLGICYDMR

SEQ ID NO: 236 - protein, 17 aa, Ratus Norvegicus
VIVVGNPANTNCLTASK

SEQ ID NO: 237 - protein, 12 aa, Ratus Norvegicus
VLDASWYSPGTR

SEQ ID NO: 238 - protein, 10 aa, Ratus Norvegicus
VLSIGDGIAR

SEQ ID NO: 239 - protein, 9 aa, Ratus Norvegicus
VLTEIIASR

SEQ ID NO: 240 - protein, 10 aa, Ratus Norvegicus
VLTPTQVMNR

SEQ ID NO: 241 - protein, 10 aa, Ratus Norvegicus
VLVAQHDAYK

SEQ ID NO: 242 - protein, 17 aa, Ratus Norvegicus
VMLGETNPADSKPGTIR

SEQ ID NO: 243 - protein, 11 aa, Ratus Norvegicus
VMVAEALDISR

SEQ ID NO: 244 - protein, 15 aa, Ratus Norvegicus
VNQIGSVTESLQACK

SEQ ID NO: 245 - protein, 8 aa, Ratus Norvegicus
VPDFSDYR

SEQ ID NO: 246 - protein, 10 aa, Ratus Norvegicus
VPGATMLLAK

SEQ ID NO: 247 - protein, 11 aa, Ratus Norvegicus
VTQSNFAVGYK

SEQ ID NO: 248 - protein, 8 aa, Ratus Norvegicus
VWVYPPEK

SEQ ID NO: 249 - protein, 15 aa, Ratus Norvegicus
WIDIHNPATNEVVGR

SEQ ID NO: 250 - protein, 11 aa, Ratus Norvegicus
WTEYGLTFTEK

SEQ ID NO: 251 - protein, 8 aa, Ratus Norvegicus
YALSVGYR

SEQ ID NO: 252 - protein, 10 aa, Ratus Norvegicus
YCTDTSIIFR

SEQ ID NO: 253 - protein, 9 aa, Ratus Norvegicus
YEWDVAEAR

SEQ ID NO: 254 - protein, 7 aa, Ratus Norvegicus
YFPVFEK

SEQ ID NO: 255 - protein, 10 aa, Ratus Norvegicus
YHPAQPLHMK

SEQ ID NO: 256 - protein, 12 aa, Ratus Norvegicus
YITPDQLADLYK

SEQ ID NO: 257 - protein, 11 aa, Ratus Norvegicus
YIVPMITVDGK

SEQ ID NO: 258 - protein, 12 aa, Ratus Norvegicus
YLAEVAAGDDKK

SEQ ID NO: 259 - protein, 7 aa, Ratus Norvegicus
YLHEVAR

SEQ ID NO: 260 - protein, 9 aa, Ratus Norvegicus
YLTVAAVFR

SEQ ID NO: 261 - protein, 14 aa, Ratus Norvegicus
YNEVLTQCCTESDK

SEQ ID NO: 262 - protein, 8 aa, Ratus Norvegicus
YNLGLDLR

SEQ ID NO: 263 - protein, 12 aa, Ratus Norvegicus
YQVDPDACFSAK

SEQ ID NO: 264 - protein, 10 aa, Ratus Norvegicus
YTALVDAEEK

SEQ ID NO: 265 - protein, 10 aa, Ratus Norvegicus
YVMFHLINFK

SEQ ID NO: 266 - protein, 10 aa, Ratus Norvegicus
YVMFHLINFK

SEQ ID NO: 267 - protein, 8 aa, Ratus Norvegicus
YYCFQGNK

SEQ ID NO: 268 - protein, 11 aa, Ratus Norvegicus
YLAEFATGNDR

SEQ ID NO: 269 - protein, 12 aa, Ratus Norvegicus
HLIPAANTGESK

SEQ ID NO: 270 - protein, 11 aa, Ratus Norvegicus
EAAENSLVAYK

SEQ ID NO: 271 - protein, 13 aa, Ratus Norvegicus
VAGMDVELTVEER

SEQ ID NO: 272 - protein, 7 aa, Ratus Norvegicus
CNFYDNK

(19) United States
(12) Patent Application Publication
(10) Pub. No.: US 2004/0053309 A1
(43) Pub. Date: Mar. 18, 2004

(54) Proteins, genes and their use for diagnosis and treatment of kidney response

(76) Inventors: Gordon Duane Holt, Oxon (GB); Michael Douglas Kelly, Oxon (GB); Sandra Jane Kennedy, Oxon (GB); Christopher Moyses, Oxon (GB)

Correspondence Address: David A. Jackson, KLAUBER & JACKSON, 4th Floor, 411 Hackensack Street, Hackensack, NJ, 07601, US

(21) Appl. No.: 10/601,837

(22) Filed: Jun. 23, 2003

Related U.S. Application Data
(63) Continuation of application No. PCT/GB01/05777, filed on Dec. 24, 2001.
(60) Provisional application No. 60/260,392, filed on Dec. 29, 2000.

(51) Int. Cl. C12Q 1/68; G01N 33/53

(52) U.S. Cl. 435/6; 435/7.1

(57) ABSTRACT

The present invention provides methods and compositions for screening, diagnosis and prognosis of kidney response, for monitoring the effectiveness of kidney response treatment, identifying patients most likely to respond to a particular therapeutic treatment and for drug development. In particular, the screening of drug candidates for their ability to induce a kidney response. Kidney Response-Associated Features (KRFs), detectable by two-dimensional electrophoresis of blood, serum, plasma or kidney tissue are described. The invention further provides Kidney Response-Associated Protein Isoforms (KRPIs) detectable in blood, serum, plasma or kidney tissue, preparations comprising isolated KRPIs, antibodies immunospecific for KRPIs, and kits comprising the aforesaid.

### INTRODUCTION

The present invention relates to the identification of proteins and protein isoforms that are associated with kidney response to toxic effectors, including its onset and development, and of genes encoding the same, and to their use for clinical screening, diagnosis, prognosis, therapy and prophylaxis, as well as for drug screening and drug development.

### BACKGROUND OF THE INVENTION

The kidney is the primary site for the excretion of endotoxic and exotoxic molecules (e.g., drugs, chemicals, etc), which are defined herein ‘toxic effectors’. All of the kidney's functions are in a state of continual flux as the organ responds to these toxic effectors. Any disruptions in the kidney's responsiveness to environmental changes can lead to serious, often life-threatening, consequences. A wide variety of toxic effectors can be disruptive to the kidney:

Chemical Poisoning

Antibacterials: aminoglycosides, vancomycin (Beringer P M; Wong-Beringer A; Rho J P, 1998, Pharmacoeconomics, 13:35-49)

Antivirals: adefovir, cidofovir (Kahn J; Lagakos S; Wulfsohn M; Cherng D; Miller M; Cherrington J; Hardy D; Beall G; Cooper R; Murphy R; Basgoz N; Ng E; Deeks S; Winslow D; Toole J J; Coakley D, 1999, JAMA 282:2305-12; Plosker G L; Noble S, 1999, Drugs 58:325-45)

Antifungals: Amphotericin B (Brogden R N; Goa K L; Coukell A J, 1998, Drugs 56:365-383)

Imaging contrast agents: iohexol, diatrizoate (Brogden R N; Goa K L; Coukelf A J, 1995, Kidney Int 47:254-61)

Nonsteroidal anti-inflammatory agents: aspirin, acetaminophen, ibuprofen (Lindeman R D, 1999, Geriatr Nephrol Urol 9:3-4)

Immunosuppressive drugs: cyclosporin A, tacrolimus (de Mattos A M; Olyaei A J; Bennett W M, 2000, Am J Kidney Dis 35:333-46)

Diabetic Nephropathy

Nephron damage due to high circulating glucose concentrations. Blood glucose reduction can delay or prevent onset of diabetic nephropathy

High Blood Pressure

High blood pressure damages the capillaries throughout the kidney

Genetic Disease

Polycystic kidney disease

Mechanical Trauma

The kidney is an architecturally complex organ composed of more than a dozen unique cell types. Kidney-disrupting toxic effectors may exclusively affect just one of these cell types, or, more commonly, may interfere with several types simultaneously. Thus, affected areas may range from highly focal to organ-wide lesions, and may spread or refocus over time. The intracellular response to toxic effectors may also change over time, for example beginning with the formation of acidic vascular inclusions and transitioning to a collagen fiber deposition over time. The following major classifications of kidney changes are defined herein as kidney responses to toxic effectors:

Nephron cell metabolic pathway modulation—Nephron cell response to toxic effectors such as drugs, chemicals, and other small molecules by the modulated synthesis of intracellular proteins such as mitochondrial or DNA repair proteins.

Glomerular/proximal tubular nephritis—Inflammation of specific kidney domains associated with antibody binding, complement fixation and/or immune cell infiltration. Chemical toxicants and autoimmune conditions are often associated with nephritis.

Glomerular/papillary necrosis—Localized cell death due to chronic damage such as that induced by high blood pressure, diabetes, and long-term insult by chemical toxicants.

Acute renal failure—Mounting cessation of blood filtration and excretion of waste products into the urine. Acute renal failure generally is caused by short duration, overwhelming insults such as chemical poisoning or mechanical injury. Acute renal failure may be reversed if the kidney damage is not serious.

Chronic renal failure—Mounting cessation of blood filtration and excretion of waste products into the urine. Chronic renal failure generally is caused by long duration, gradual insults such as diabetes or high blood pressure. Chronic renal failure is rarely reversible.

End-stage renal disease—Complete cessation of blood filtration and excretion of waste products into the urine. Patients must undergo dialysis or kidney transplant to survive.

Given the high degree of variability in its causes and classifications, there currently is no specific measure of the kidney response to toxic effectors. The following list outlines currently validated measures of kidney homeostasis:

Nonintrusive assays

serum creatinine and blood urea nitrogen (BUN) levels; creatinine clearance rates

urine creatinine and protein levels

soft tissue imaging including sonography, magnetic resonance imaging, computed tomography

radioisotope metabolic labeling

Intrusive Assays

needle biopsy

surgery

All of the current measures of kidney homeostasis suffer from one or more significant limitations. For example, the non-intrusive assays show poor correlation with kidney histopathology and generally provide no prospective measure of how the kidney will further change over time. The intrusive kidney homeostasis kidney assays also suffer from the limitation that they present significant risk to the test subject. Therefore, they cannot be employed unless the subject's life is already under serious threat in the case of human testing. In addition, the intrusive assays require time-consuming and costly interpretation by expert pathologists and may provide ambiguous results if the tissue changes are not homogeneous across the kidney relative to the sample examined.

The current measures of kidney homeostasis are also severely limited in their usefulness in facilitating the development of new treatments for human disease.

The currently available kidney homeostasis measures also suffer from a poor correlation between animal study results and kidney responses in humans. The noninvasive measures of kidney homeostasis are particularly difficult to correlate in response to toxic effectors compared to humans. The utility of animal-based invasive measures of kidney homeostasis also are quite limited in that they pose unethical risk if they were to be administered during human treatment trials.

A variety of anecdotal studies have shown alterations in the levels of proteins in the kidney or serum in response to toxic effectors. However, we are aware of no systematic effort to correlate these observations with clinically relevant features of kidney damage such as functional assessments, or the rate at which damage is proceeding or recovering to identify statistically significant changes in protein levels.

Due to the costly and time consuming nature of existing, often ambiguous, tests it would be highly desirable to measure a substance or substances in samples of blood or kidney that would lead to a positive diagnosis of kidney response or that would help to exclude kidney response from a differential diagnosis.

The development of new pharmaceutical compositions and/or treatment regimens directed towards the treatment or prophylaxis of diseases, infectious or otherwise, relies heavily on the ability to screen candidate compounds for possible toxic or pathological responses, e.g. kidney response. In drug development, a putative drug is tested in a battery of assays and in laboratory animals to ascertain its safety (i.e. lack of toxicity) and effectiveness. The costs associated with the development of new pharmaceutical reagents are ever increasing, particularly when new compositions enter clinical trials. It is not unheard of for promising pharmaceutical candidates to pass the appropriate laboratory tests and enter the expensive stage of animal and human clinical trials, only to present toxic or pathologic effects in the in vivo setting for the targeted patient, normally humans. The elimination of previously-promising drug candidates at such a late stage in product development is a major factor in the high costs of new effective drugs which ultimately do pass the final clinical trials.

Therefore, a need exists to identify kidney response-associated proteins as sensitive and specific biomarkers for the diagnosis, to assess severity and predict the outcome of kidney response in response subjects and, in particular, to allow the screening of drug candidates for their ability to induce a kidney response. Additionally, there is a clear need for new therapeutic agents for kidney response that work quickly, potently, specifically, and with fewer side effects.

### SUMMARY OF THE INVENTION

The present invention provides methods and compositions for clinical screening, diagnosis, prognosis, therapy and prophylaxis of kidney response, in particular, the screening of drug candidates for their ability to induce a kidney response. For monitoring the effectiveness of kidney response treatment, for selecting participants in clinical trials, for identifying patients most likely to respond to a particular therapeutic treatment and for screening and development of drugs for treatment of kidney response.

A first aspect of the invention provides methods for diagnosis of kidney response that comprise analyzing a sample of blood or kidney tissue by two-dimensional electrophoresis to detect the presence or level of at least one Kidney Response-Associated Feature (KRF), e.g., one or more of the KRFs disclosed herein or any combination thereof. These methods are also suitable for clinical screening, prognosis, monitoring the results of therapy, identifying patients most likely to respond to a particular therapeutic treatment, for drug screening and development, and identification of new targets for drug treatment.

A second aspect of the invention provides methods for diagnosis of kidney response that comprise detecting in a sample of blood or kidney tissue the presence or level of at least one Kidney Response-Associated Protein Isoform (KRPI), e.g., one or more of the KRPIs disclosed herein or any combination thereof. These methods are also suitable for clinical screening, prognosis, monitoring the results of therapy, identifying patients most likely to respond to a particular therapeutic treatment, drug screening and development, and identification of new targets for drug treatment.

A third aspect of the invention provides antibodies, e.g. monoclonal and polyclonal chimeric (bispecific) antibodies capable antibodies of immunospecific binding to a KRPI, e.g., a KRPI disclosed herein.

A fourth aspect of the invention provides a preparation comprising an isolated KRPI, i.e., a KRPI substantially free from proteins or protein isoforms having a significantly different isoelectric point or a significantly different apparent molecular weight from the KRPI.

A fifth aspect of the invention provides kits that may be used in the above recited methods and that may comprise single or multiple preparations, or antibodies, together with other reagents, labels, substrates, if needed, and directions for use. The kits may be used for diagnosis of disease, or may be assays for the identification of new diagnostic and/or therapeutic agents.

A sixth aspect of the invention provides methods of treating kidney response, comprising administering to a subject a therapeutically effective amount of an agent that modulates (e.g., upregulates or downregulates) the expression or activity (e.g. enzymatic or binding activity), or both, of a KRF or KRPI in subjects having kidney response, in order to prevent or delay the onset or development of kidney response, to prevent or delay the progression of kidney response, or to ameliorate the symptoms of kidney response.

A seventh aspect of the invention provides methods of screening for agents that modulate (e.g., upregulate or downregulate) a characteristic of, e.g., the expression or the enzymatic or binding activity, of a KRF, a KRPI, a KRPI analog, or a KRPI-related polypeptide. This aspect of the invention being particularly useful in determining the ability of drug candidates to induce a kidney response.

### BRIEF DESCRIPTION OF THE FIGURES

FIG. 1 is a flow chart depicting the characterization of a KRF and relationship of a KRF and KRPI. A KRF may be further characterized as or by a KRPI having a particular peptide sequence associated with its pI and MW. As depicted herein, a KRF may comprise one or more KRPIs, which have indistinguishable pI and MWs using the Preferred Technology, but which comprise distinct peptide sequences. The peptide sequence(s) of the KRPI can be utilized to search database(s) for previously-identified proteins comprising such peptide sequence(s). In some instances, it can be ascertained whether a commercially-available antibody exists which may recognize the previously identified protein and/or variant thereof. It should be noted that the KRPI may correspond to the previously-identified protein, be a variant of the previously-identified protein, or be a previously unknown protein.

FIG. 2 is an image obtained from 2-dimensional electrophoresis of rat kidney cortex, which has been annotated to identify twelve landmark features.

FIG. 3 is an image obtained from 2-dimensional electrophoresis of rat blood, which has been annotated to identify ten landmark features.

### DETAILED DESCRIPTION OF THE INVENTION

The present invention described in detail below provides methods, compositions and kits useful, e.g., for screening, diagnosis and treatment of kidney response in a mammalian subject, and for drug screening and drug development. When the invention is used to determine the ability of drug candidiates to induce a kidney response, the body tissue or body fluid which is analysed for the presence or level of at least one kidney response feature is preferably from a non-human mammal. The non-human mammal is preferably one in which the induction of a kidney response by endogenous and/or exogenous effector agents is predictive of the induction of such a response in humans. The rat is a particularly suitable mammal for use in this aspect of the invention.

The invention also encompasses the administration of therapeutic compositions to a mammalian subject to treat or prevent kidney response. The mammalian subject may be a non-human mammal, but is preferably human, more preferably a human adult, i.e. a human subject at least 21 (more preferably at least 35, at least 50, at least 60, at least 70, or at least 80) years old. For clarity of disclosure, and not by way of limitation, the invention will be described with respect to the analysis of blood samples and to kidney tissue samples. However, as one skilled in the art will appreciate, the assays and techniques described below can be applied to other types of samples, including a body fluid (e.g. spinal fluid, plasma, saliva or urine), a tissue sample from a subject at risk of having or developing kidney response (e.g. a biopsy such as a kidney biopsy) or homogenate thereof. The methods and compositions of the present invention are useful for screening, diagnosis and prognosis of a living subject, but may also be used for postmortem diagnosis in a subject, for example, to identify family members of the subject who are at risk of developing the same disease.

Definitions

“Kidney Response” refers to and includes alteration in kidney function, and/or other organ or cellular function and/or any condition, that comes about from the interaction of the kidney with toxic effectors. Kidney response includes but is not limited to any aspect or phase of nephron cell metabolic pathway modulation, glomerular/proximal tubular nephritis, glomerular/papillary necrosis, acute renal failure, chronic renal failure, and end-stage renal disease. ‘toxic effectors’ include but are not limited to xenobiotics, chemical poisoning, diabetic nephropathy, high blood pressure, genetic disease, mechanical trauma, viruses and other biological agents.

“Feature” refers to a spot detected in a 2D gel, and the term “Kidney Response—Associated Feature” (KRF) refers to a feature that is differentially present in a sample from a subject having kidney response compared with a sample from a subject free from kidney response. A feature or spot detected in a 2D gel is characterized by its isoelectric point (pI) and molecular weight (MW) as determined by 2D gel electrophoresis, particularly utilizing the Preferred Technology described herein. As used herein, a feature is “differentially present” in a first sample with respect to a second sample when a method for detecting the said feature (e.g., 2D electrophoresis) gives a different signal when applied to the first and second samples. A KRF, (or a protein isoform, i.e. KRPI, as defined infra) is “increased” in the first sample with respect to the second if the method of detection indicates that the KRF, or KRPI is more abundant in the first sample than in the second sample, or if the KRF, or KRPI is detectable in the first sample and substantially undetectable in the second sample. Conversely, a KRF, or KRPI is “decreased” in the first sample with respect to the second if the method of detection indicates that the KRF, or KRPI is less abundant in the first sample than in the second sample or if the KRF, or KRPI is undetectable in the first sample and detectable in the second sample.

Preferably, the relative abundance of a feature in two samples is determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed below, or (c) more preferably to the total signal detected as the sum of each of all proteins in the sample.

Secondly, the normalized signal for the feature in one sample or sample set is compared with the normalized signal for the same feature in another sample or sample set in order to identify features that are “differentially present” in the first sample (or sample set) with respect to the second.

“Fold change” includes “fold increase” and “fold decrease” and refers to the relative increase or decrease in abundance of a KRF or the relative increase or decrease in expression or activity of a polypeptide (e.g. a KRPI, as defined infra.) in a first sample or sample set compared to a second sample (or sample set). A KRF or polypeptide fold change may be measured by any technique known to those of skill in the art, albeit the observed increase or decrease will vary depending upon the technique used. Preferably, fold change is determined herein as described in the Examples infra.

“Kidney Response-Associated Protein Isoform” (KRPI) refers to a protein isoform that is differentially present in a sample from a subject having kidney response compared with a sample from a subject free from any kidney response or that is differentially present in a sample from a subject having one or more particular kidney response compared with a sample from a subject free from such one or more particular kidney response or having a distinct kidney response. As used herein, a KRPI is “differentially present” in a first sample with respect to a second sample when a method for detecting the said feature, (e.g., 2D electrophoresis or immunoassay) gives a different signal when applied to the first and second samples (refer to KRF definition).

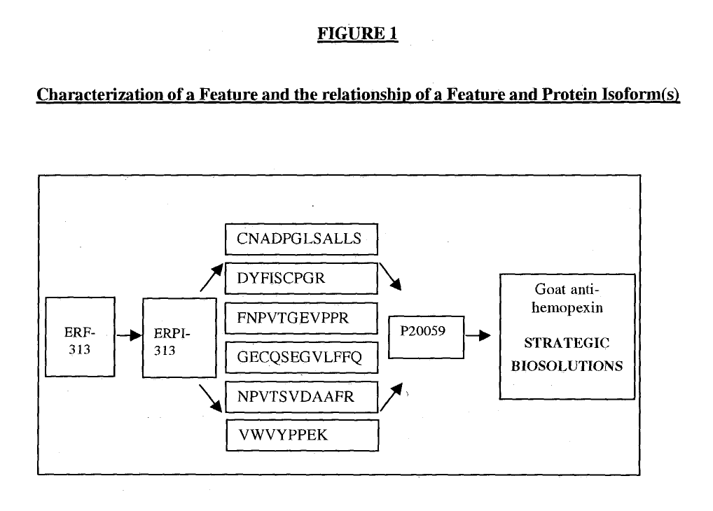

A KRPI is characterised by one or more peptide sequences of which it is comprised, and further by a pI and MW, preferably determined by 2D electrophoresis, particularly utilising the Preferred Technology as described herein. Typically, KRPIs are identified or characterized by the amino acid sequencing of KRFs (FIG. 1).

FIG. 1 is a flow chart depicting the characterization of a KRF and relationship of a KRF and KRPI(s). A KRF may be further characterized as or by a KRPI having a particular peptide sequence associated with its pI and MW. As depicted herein, a KRF may comprise one or more KRPIs, which have indistinguishable pI and MWs using the Preferred Technology, but which comprise distinct peptide sequences. The peptide sequence(s) of the KRPI can be utilized to search database(s) for previously-identified proteins comprising such peptide sequence(s). In some instances, it can be ascertained whether a commercially-available antibody exists which may recognize the previously identified protein and/or variant thereof. It should be noted that the KRPI may correspond to the previously-identified protein, be a variant of the previously-identified protein, or be a previously unknown protein.

“Variant” as used herein refers to a polypeptide which is a member of a family of polypeptides that are encoded by a single gene or from a gene sequence within a family of related genes and which differ in their pI or MW, or both. Such variants can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation).

“Modulate” in reference to expression or activity of a KRF, KRPI or a KRPI-related polypeptide refers to any change, e.g., upregulation or downregulation, increase or decrease, of the expression or activity of the KRF, KRPI or a KRPI-related polypeptide. Those skilled in the art, based on the present disclosure, will understand that such modulation can be determined by assays known to those of skill in the art.

“KRPI analog” refers to a polypeptide that possesses similar or identical function(s) as a KRPI but need not necessarily comprise an amino acid sequence that is similar or identical to the amino acid sequence of the KRPI, or possess a structure that is similar or identical to that of the KRPI. As used herein, an amino acid sequence of a polypeptide is “similar” to that of a KRPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the KRPI; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the KRPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the KRPI. As used herein, a polypeptide with “similar structure” to that of a KRPI refers to a polypeptide that has a similar secondary, tertiary or quarternary structure as that of the KRPI. The structure of a polypeptide can determined by methods known to those skilled in the art, including but not limited to, X-ray crystallography, nuclear magnetic resonance, and crystallographic electron microscopy.

“KRPI fusion protein” refers to a polypeptide that comprises (i) an amino acid sequence of a KRPI, a KRPI fragment, a KRPI-related polypeptide or a fragment of a KRPI-related polypeptide and (ii) an amino acid sequence of a heterologous polypeptide (i.e., a non-KRPI, non-KRPI fragment or non-KRPI-related polypeptide).

“KRPI homolog” refers to a polypeptide that comprises an amino acid sequence similar to that of a KRPI but does not necessarily possess a similar or identical function as the KRPI.

“KRPI ortholog” refers to a non-rat polypeptide that (i) comprises an amino acid sequence similar to that of a KRPI and (ii) possesses a similar or identical function to that of the KRPI. It will be appreciated that the specific KRPIs identified in the description were derived from the rat. The skilled person will recognise that in various aspects of the invention it will be necessary to substitute the rat KRPI for the KRPI ortholog from another mammal e.g. a human. KRPI orthologs can be identified using techniques well known to those skilled in the art for example using homology searching e.g. as described below in relation to the determination of percent identitiy of two amino acid sequences. The similarity between the KRPIs identified and their human orthologs is on average 85% (S.E.M.=2.4) allowing for conservative substitutions (see section 5.7). It will be appreciated that in various aspects of the claimed invention, e.g. methods of treatment, it will be necessary to substitute a KRPI with a KRPI ortholog depending on the identity of the mammal to be treated.

“KRPI-related polypeptide” refers to a KRPI homolog, a KRPI analog, a variant of KRPI, a KRPI ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.)

“Derivative” refers to a polypeptide that comprises an amino acid sequence of a second polypeptide that has been altered by the introduction of amino acid residue substitutions, deletions or additions. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a KRPI may or may not possess a functional activity of the second polypeptide.

The “percent identity” of two amino acid sequences or of two nucleic acid sequences can be or is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res.25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

“Diagnosis” refers to diagnosis, prognosis, monitoring, characterizing, selecting patients, including participants in clinical trials, and identifying patients at risk for or having a particular disorder or those most likely to respond to a particular therapeutic treatment, or for assessing or monitoring a patient's response to a particular therapeutic treatment.

“Treatment” refers to therapy, prevention and prophylaxis and particularly refers to the administration of medicine or the performance of medical procedures with respect to a patient, for either prophylaxis (prevention) or to cure the infirmity or malady in the instance where the patient is afflicted.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

‘Blood’ as used herein includes serum and plasma. ‘Serum’ refers to the supernatant fluid produced by clotting and centrifugal sedimentation of a blood sample. ‘Plasma’ refers to the supernatant fluid produced by inhibition of clotting (for example, by citrate or EDTA) and centrifugal sedimentation of a blood sample.

As used herein, the term ‘kidney tissue’ refers to the cell layers that line the kidney.

Kidney Response-Associated Features (KRFs)

In one aspect of the invention, two-dimensional electrophoresis is used to analyze blood or kidney tissue from a subject, preferably a living subject, in order to detect or quantify the expression of one or more Kidney Response-Associated Features (KRFs) for screening, prevention or diagnosis of kidney response, to determine the prognosis of a subject having kidney response, to monitor progression of kidney response, to monitor the effectiveness of kidney response therapy, for identifying patients most likely to respond to a particular therapeutic treatment, or for drug development, and, in particular, to determine the potential for drug candidates to induce a kidney response.

By way of example and not of limitation, using the Preferred Technology, a number of samples from subjects having kidney response and samples from subjects free from kidney response are separated by two-dimensional electrophoresis, and the fluorescent digital images of the resulting gels are matched to a chosen representative primary master gel image. This process allows any gel feature, characterised by its pI and MW, to be identified and examined on any gel of the study. In particular, the amount of protein present in a given feature can be measured in each gel; this feature abundance can be averaged amongst gels from similar samples (e.g. gels from samples from subjects having kidney response). Finally, statistical analyses can be conducted on the thus created sample sets, in order to compare 2 or more sample sets to each other.

As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97 GB3307 (published as WO 98/23950) and in U.S. Pat. No. 6,064,754, each of which is incorporated herein by reference in its entirety with particular reference to the protocol at pages 23-35. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A preferred scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

As used herein, the term “feature” refers to a spot detected in a 2D gel, and the term “Kidney Response-Associated Feature” (KRF) refers to a feature that is differentially present in a sample (e.g. a sample of tissue) from a subject having kidney response compared with a sample (e.g. a sample of tissue) from a subject free from kidney response.

In accordance with an aspect of the present invention, the KRFs disclosed herein have been identified by comparing blood or kidney tissue from subjects having kidney response against blood or kidney tissue from subjects free from kidney response. In the experiments conducted on samples of kidney tissue, comparisons were made between subjects free from kidney response and subjects having kidney response induced by the following dosage levels of gentamicin: 0.1, 1.0, 10, 40 or 60 mg/kg/day, after two time points (i.e. after day 8 and day 22 of the treatment) as described in the Examples infra. In the experiments conducted on samples of blood, comparisons were made between subjects free from kidney response and subjects having kidney response induced by a 40 mg/kg/day dosage level of gentamicin taken after 8 days of the treatment as described in the Examples infra.

Four groups of KRFs have been identified through the methods and apparatus of the Preferred Technology. The first group consists of KRFs that are decreased in the kidney tissue of subjects having kidney response as compared with the kidney tissue of subjects free from kidney response. These KRFs can be described by apparent molecular weight (MW) and isoelectric point (pI) as provided in Table I.

TABLE I
KRFs Decreased in Tissue of Subjects Having Kidney Response

| KRF | pI | MW (Da) |
|---|---|---|
| KRF-1 | 5.1 | 43,557 |
| KRF-2 | 7.3 | 35,621 |
| KRF-3 | 4.9 | 39,951 |
| KRF-4 | 5.1 | 101,577 |
| KRF-5 | 4.9 | 33,363 |
| KRF-6 | 5.3 | 67,007 |
| KRF-7 | 5.4 | 28,601 |
| KRF-8 | 5.0 | 24,350 |
| KRF-9 | 6.5 | 37,386 |
| KRF-10 | 7.2 | 46,674 |
| KRF-11 | 5.4 | 41,863 |
| KRF-12 | 5.1 | 63,105 |
| KRF-13 | 5.4 | 21,765 |
| KRF-14 | 6.8 | 12,639 |
| KRF-15 | 5.0 | 25,902 |
| KRF-16 | 5.2 | 21,913 |
| KRF-17 | 5.9 | 33,673 |
| KRF-18 | 5.2 | 81,710 |
| KRF-19 | 7.0 | 21,399 |
| KRF-20 | 6.1 | 26,255 |
| KRF-21 | 5.4 | 80,627 |
| KRF-22 | 5.2 | 39,194 |
| KRF-23 | 7.2 | 20,698 |
| KRF-24 | 8.0 | 23,594 |
| KRF-25 | 5.3 | 20,828 |
| KRF-26 | 7.8 | 31,756 |
| KRF-27 | 4.9 | 31,623 |
| KRF-28 | 5.6 | 42,298 |
| KRF-29 | 5.6 | 38,745 |
| KRF-30 | 5.5 | 17,155 |
| KRF-31 | 5.1 | 65,723 |
| KRF-32 | 5.7 | 18,083 |
| KRF-33 | 5.2 | 18,968 |
| KRF-34 | 5.6 | 35,836 |
| KRF-35 | 5.7 | 34,167 |
| KRF-36 | 5.6 | 58,058 |
| KRF-37 | 4.7 | 14,017 |
| KRF-38 | 5.2 | 16,833 |
| KRF-39 | 5.7 | 25,316 |
| KRF-40 | 5.3 | 80,900 |
| KRF-41 | 5.8 | 43,502 |
| KRF-42 | 5.8 | 39,836 |
| KRF-43 | 6.8 | 21,939 |
| KRF-44 | 5.3 | 41,834 |
| KRF-45 | 7.1 | 23,849 |
| KRF-46 | 7.3 | 23,602 |
| KRF-47 | 6.1 | 37,336 |
| KRF-48 | 5.0 | 59,778 |
| KRF-49 | 6.1 | 42,207 |
| KRF-50 | 7.7 | 49,647 |
| KRF-51 | 6.9 | 34,872 |
| KRF-52 | 7.1 | 14,187 |
| KRF-53 | 6.7 | 28,930 |
| KRF-54 | 7.7 | 26,100 |
| KRF-55 | 5.0 | 18,626 |
| KRF-56 | 6.0 | 43,514 |
| KRF-57 | 6.8 | 11,462 |
| KRF-58 | 5.9 | 80,299 |
| KRF-59 | 5.7 | 27,218 |
| KRF-60 | 5.3 | 20,135 |
| KRF-61 | 4.7 | 12,754 |
| KRF-62 | 6.0 | 22,665 |
| KRF-63 | 6.4 | 32,486 |
| KRF-64 | 6.5 | 38,483 |
| KRF-65 | 5.9 | 38,705 |
| KRF-66 | 6.9 | 22,363 |
| KRF-67 | 7.6 | 45,480 |
| KRF-68 | 6.1 | 49,829 |
| KRF-69 | 7.4 | 21,692 |
| KRF-70 | 7.7 | 20,347 |
| KRF-71 | 6.5 | 23,591 |
| KRF-72 | 7.6 | 37,026 |
| KRF-73 | 7.3 | 27,831 |
| KRF-74 | 5.0 | 11,914 |
| KRF-75 | 5.3 | 59,546 |
| KRF-76 | 7.0 | 24,556 |
| KRF-77 | 6.2 | 53,362 |
| KRF-78 | 8.3 | 33,363 |
| KRF-79 | 5.7 | 22,899 |
| KRF-80 | 6.7 | 13,087 |
| KRF-81 | 5.2 | 64,776 |
| KRF-82 | 5.7 | 43,557 |
| KRF-83 | 7.1 | 20,828 |
| KRF-84 | 6.3 | 21,397 |
| KRF-85 | 7.3 | 18,969 |
| KRF-86 | 5.6 | 11,175 |
| KRF-87 | 6.0 | 62,820 |
| KRF-88 | 7.7 | 18,953 |
| KRF-89 | 6.5 | 21,473 |
| KRF-90 | 8.5 | 16,508 |
| KRF-91 | 6.0 | 13,898 |
| KRF-92 | 5.0 | 58,397 |
| KRF-93 | 5.8 | 38,705 |
| KRF-94 | 5.3 | 16,190 |
| KRF-95 | 6.2 | 70,946 |
| KRF-96 | 8.0 | 27,637 |
| KRF-97 | 5.4 | 12,570 |
| KRF-98 | 6.1 | 20,618 |
| KRF-99 | 5.2 | 36,031 |
| KRF-100 | 7.6 | 24,966 |
| KRF-101 | 7.7 | 24,269 |
| KRF-102 | 5.6 | 25,071 |
| KRF-103 | 5.9 | 45,139 |
| KRF-104 | 7.1 | 26,948 |
| KRF-105 | 9.4 | 34,066 |
| KRF-106 | 7.5 | 31,908 |
| KRF-107 | 7.1 | 12,919 |
| KRF-108 | 7.9 | 12,011 |
| KRF-109 | 6.1 | 55,825 |
| KRF-110 | 6.8 | 20,454 |
| KRF-111 | 5.8 | 18,533 |
| KRF-112 | 5.9 | 36,106 |
| KRF-113 | 7.1 | 35,304 |
| KRF-114 | 5.0 | 19,067 |
| KRF-115 | 7.7 | 40,678 |
| KRF-116 | 6.9 | 34,066 |
| KRF-117 | 6.8 | 10,596 |
| KRF-118 | 6.1 | 37,985 |
| KRF-119 | 7.6 | 17,845 |
| KRF-120 | 5.7 | 40,982 |
| KRF-121 | 4.8 | 46,728 |
| KRF-122 | 5.3 | 11,763 |
| KRF-123 | 5.0 | 44,701 |
| KRF-124 | 5.6 | 33,463 |
| KRF-125 | 7.2 | 22,363 |
| KRF-126 | 5.2 | 64,776 |
| KRF-127 | 4.6 | 38,483 |
| KRF-128 | 7.5 | 28,930 |
| KRF-129 | 6.3 | 19,571 |
| KRF-130 | 7.3 | 23,929 |
| KRF-131 | 7.9 | 37,143 |
| KRF-132 | 7.0 | 36,051 |
| KRF-133 | 4.6 | 27,322 |
| KRF-134 | 5.6 | 24,011 |
| KRF-135 | 5.2 | 31,880 |
| KRF-136 | 4.5 | 13,709 |
| KRF-137 | 6.4 | 40,102 |
| KRF-138 | 7.6 | 35,652 |
| KRF-139 | 7.1 | 27,742 |
| KRF-140 | 7.1 | 34,055 |
| KRF-141 | 8.6 | 33,255 |
| KRF-142 | 6.0 | 78,163 |
| KRF-143 | 7.7 | 26,909 |
| KRF-144 | 6.8 | 23,369 |
| KRF-145 | 7.2 | 22,977 |
| KRF-146 | 7.9 | 30,881 |
| KRF-147 | 5.8 | 25,350 |
| KRF-148 | 6.2 | 51,783 |
| KRF-149 | 7.4 | 51,414 |
| KRF-150 | 7.4 | 39,580 |
| KRF-151 | 6.5 | 59,042 |
| KRF-152 | 5.2 | 57,842 |
| KRF-153 | 5.7 | 55,401 |
| KRF-154 | 8.0 | 41,346 |
| KRF-155 | 5.4 | 75,406 |
| KRF-156 | 5.3 | 27,323 |
| KRF-157 | 7.5 | 27,600 |
| KRF-158 | 5.5 | 67,349 |
| KRF-159 | 6.9 | 40,414 |
| KRF-160 | 5.1 | 34,378 |
| KRF-161 | 7.9 | 48,455 |
| KRF-162 | 6.9 | 54,354 |
| KRF-163 | 6.0 | 79,341 |
| KRF-164 | 5.9 | 36,047 |
| KRF-165 | 6.3 | 23,223 |
| KRF-166 | 6.0 | 55,886 |
| KRF-167 | 5.0 | 38,259 |
| KRF-168 | 8.9 | 24,933 |
| KRF-169 | 5.5 | 17,857 |
| KRF-170 | 8.8 | 26,806 |
| KRF-171 | 5.5 | 48,755 |
| KRF-172 | 5.6 | 38,758 |
| KRF-173 | 8.3 | 20,702 |
| KRF-174 | 5.8 | 56,049 |
| KRF-175 | 6.0 | 72,833 |
| KRF-176 | 6.9 | 53,667 |
| KRF-177 | 5.2 | 60,527 |
| KRF-178 | 6.6 | 22,591 |
| KRF-179 | 8.7 | 27,848 |
| KRF-180 | 5.5 | 57,804 |

The second group consists of KRFs that are increased in the kidney tissue of subjects having kidney response as compared with the kidney tissue of subjects free from kidney response. These KRFs can be described by MW and pI as provided in Table II.

TABLE II
KRFs Increased in Kidney Tissue of Subjects
Having Kidney Response

| KRF | pI | MW (Da) |
|---|---|---|
| KRF-8 | 5.0 | 24,350 |
| KRF-22 | 5.2 | 39,194 |
| KRF-27 | 4.9 | 31,623 |
| KRF-28 | 5.6 | 42,298 |
| KRF-30 | 5.5 | 17,155 |
| KRF-36 | 5.6 | 58,058 |
| KRF-38 | 5.2 | 16,833 |
| KRF-47 | 6.1 | 37,336 |
| KRF-51 | 6.9 | 34,872 |
| KRF-54 | 7.7 | 26,100 |
| KRF-67 | 7.6 | 45,480 |
| KRF-68 | 6.1 | 49,829 |
| KRF-97 | 5.4 | 12,570 |
| KRF-111 | 5.8 | 18,533 |
| KRF-112 | 5.9 | 36,106 |
| KRF-116 | 6.9 | 34,066 |
| KRF-140 | 7.1 | 34,055 |
| KRF-141 | 8.6 | 33,255 |
| KRF-142 | 6.0 | 78,163 |
| KRF-144 | 6.8 | 23,369 |
| KRF-145 | 7.2 | 22,977 |
| KRF-147 | 5.8 | 25,350 |
| KRF-148 | 6.2 | 51,783 |
| KRF-149 | 7.4 | 51,414 |
| KRF-150 | 7.4 | 39,580 |
| KRF-151 | 6.5 | 59,042 |
| KRF-158 | 5.5 | 67,349 |
| KRF-162 | 6.9 | 54,354 |
| KRF-181 | 8.1 | 19,167 |
| KRF-182 | 5.6 | 49,449 |
| KRF-183 | 7.9 | 34,066 |
| KRF-184 | 6.2 | 45,875 |
| KRF-185 | 5.7 | 44,444 |
| KRF-186 | 6.2 | 35,095 |
| KRF-187 | 6.3 | 23,924 |
| KRF-188 | 6.3 | 42,667 |
| KRF-189 | 7.5 | 37,358 |
| KRF-190 | 4.9 | 35,233 |
| KRF-191 | 6.4 | 56,575 |
| KRF-192 | 6.8 | 22,439 |
| KRF-193 | 5.9 | 94,481 |
| KRF-194 | 7.0 | 27,848 |
| KRF-195 | 6.9 | 35,471 |
| KRF-196 | 4.7 | 26,603 |
| KRF-197 | 6.0 | 24,011 |
| KRF-198 | 6.8 | 70,766 |
| KRF-199 | 6.1 | 50,793 |
| KRF-200 | 6.1 | 31,963 |
| KRF-201 | 6.0 | 46,540 |
| KRF-202 | 5.5 | 31,104 |
| KRF-203 | 7.5 | 30,601 |
| KRF-204 | 5.2 | 40,414 |
| KRF-205 | 7.1 | 81,188 |
| KRF-206 | 7.6 | 54,603 |
| KRF-207 | 7.5 | 81,314 |
| KRF-208 | 4.8 | 15,906 |
| KRF-209 | 5.7 | 95,301 |
| KRF-210 | 8.0 | 35,549 |
| KRF-211 | 6.3 | 64,776 |
| KRF-212 | 5.7 | 67,595 |
| KRF-213 | 8.0 | 30,983 |
| KRF-214 | 6.1 | 51,951 |
| KRF-215 | 8.2 | 27,487 |
| KRF-216 | 5.6 | 54,508 |
| KRF-217 | 5.7 | 64,234 |
| KRF-218 | 5.9 | 48,123 |
| KRF-219 | 7.4 | 13,463 |
| KRF-220 | 6.5 | 12,044 |
| KRF-221 | 7.7 | 57,174 |
| KRF-222 | 7.5 | 57,015 |
| KRF-223 | 6.7 | 48,914 |
| KRF-224 | 7.7 | 48,686 |
| KRF-225 | 6.0 | 50,369 |
| KRF-226 | 6.2 | 49,593 |
| KRF-227 | 7.5 | 60,995 |
| KRF-228 | 6.3 | 46,688 |
| KRF-229 | 7.5 | 22,173 |
| KRF-230 | 9.0 | 29,375 |
| KRF-231 | 5.8 | 53,501 |
| KRF-232 | 7.1 | 40,809 |
| KRF-233 | 5.5 | 68,054 |
| KRF-234 | 4.9 | 18,919 |
| KRF-235 | 7.1 | 43,682 |
| KRF-236 | 5.5 | 13,445 |
| KRF-237 | 9.1 | 23,172 |
| KRF-238 | 7.6 | 60,624 |
| KRF-239 | 7.8 | 59,197 |
| KRF-240 | 7.5 | 22,637 |
| KRF-241 | 5.3 | 73,537 |
| KRF-242 | 7.6 | 69,306 |
| KRF-243 | 5.5 | 34,330 |
| KRF-244 | 6.8 | 63,473 |
| KRF-245 | 4.7 | 43,086 |
| KRF-246 | 6.3 | 35,903 |
| KRF-247 | 7.3 | 59,544 |
| KRF-248 | 4.8 | 18,268 |
| KRF-249 | 5.4 | 70,401 |
| KRF-250 | 7.6 | 59,990 |
| KRF-251 | 7.0 | 53,029 |
| KRF-252 | 4.9 | 53,963 |
| KRF-253 | 9.6 | 48,151 |
| KRF-254 | 6.7 | 87,067 |
| KRF-255 | 4.8 | 12,818 |
| KRF-256 | 5.3 | 13,604 |
| KRF-257 | 4.7 | 12,867 |
| KRF-258 | 5.9 | 16,238 |
| KRF-259 | 5.6 | 86,368 |
| KRF-260 | 5.5 | 58,378 |
| KRF-261 | 5.4 | 47,412 |
| KRF-262 | 7.8 | 23,749 |
| KRF-263 | 7.7 | 42,563 |
| KRF-264 | 5.4 | 31,429 |
| KRF-265 | 6.1 | 43,075 |
| KRF-266 | 5.5 | 23,258 |
| KRF-267 | 5.6 | 28,492 |
| KRF-268 | 5.7 | 21,058 |
| KRF-269 | 6.0 | 38,864 |
| KRF-270 | 6.7 | 47,112 |
| KRF-271 | 6.9 | 30,062 |
| KRF-272 | 6.1 | 40,034 |
| KRF-273 | 4.7 | 31,342 |
| KRF-274 | 5.6 | 27,218 |
| KRF-275 | 4.9 | 21,618 |
| KRF-276 | 6.5 | 60,624 |
| KRF-277 | 4.6 | 37,808 |
| KRF-278 | 7.2 | 78,547 |
| KRF-279 | 5.8 | 46,599 |
| KRF-280 | 6.5 | 43,914 |
| KRF-281 | 4.9 | 30,750 |
| KRF-282 | 4.7 | 15,768 |
| KRF-283 | 5.0 | 28,061 |
| KRF-284 | 6.0 | 26,976 |
| KRF-285 | 5.8 | 46,740 |
| KRF-286 | 5.6 | 22,363 |
| KRF-287 | 5.5 | 36,325 |
| KRF-288 | 5.1 | 40,583 |
| KRF-289 | 5.5 | 20,307 |

The third group consists of KRFs that are decreased in the blood of subjects having kidney response as compared with the blood of subjects free from kidney response. These KRFs can be described by apparent molecular weight (MW) and isoelectric point (pI) as provided in Table III.

TABLE III
KRFs Decreased in Blood of Subjects Having Kidney Response

| KRF | PI | MW (Da) |
|---|---|---|
| KRF-290 | 5.3 | 124,107 |
| KRF-291 | 8.7 | 69,580 |
| KRF-292 | 7.3 | 81,357 |
| KRF-293 | 5.6 | 136,203 |
| KRF-294 | 5.7 | 135,486 |
| KRF-295 | 5.7 | 123,856 |
| KRF-296 | 5.3 | 99,803 |
| KRF-297 | 5.3 | 23,260 |
| KRF-298 | 7.0 | 87,673 |
| KRF-299 | 4.8 | 52,986 |
| KRF-300 | 6.1 | 134,812 |
| KRF-301 | 4.9 | 52,180 |
| KRF-302 | 4.8 | 53,467 |
| KRF-303 | 5.0 | 77,747 |
| KRF-304 | 6.9 | 53,475 |
| KRF-305 | 7.2 | 50,919 |
| KRF-306 | 4.8 | 78,125 |
| KRF-307 | 6.3 | 136,964 |
| KRF-308 | 4.8 | 59,584 |
| KRF-309 | 6.8 | 49,184 |
| KRF-310 | 5.6 | 95,157 |
| KRF-311 | 5.3 | 114,923 |
| KRF-312 | 5.7 | 17,513 |
| KRF-313 | 4.9 | 53,018 |

The fourth group consists of KRFs that are increased in the blood of subjects having kidney response as compared with the blood of subjects free from kidney response. These KRFs can be described by apparent molecular weight (MW) and isoelectric point (pI) as provided in Table IV.

TABLE IV
KRFs Increased in Blood of Subjects Having Kidney Response

| KRF | pI | MW (Da) |
|---|---|---|
| KRF-314 | 5.7 | 35,921 |
| KRF-315 | 6.2 | 88,662 |
| KRF-316 | 5.4 | 65,170 |
| KRF-317 | 6.3 | 87,681 |
| KRF-318 | 5.6 | 33,267 |
| KRF-319 | 4.7 | 33,621 |
| KRF-320 | 6.1 | 89,623 |
| KRF-321 | 6.0 | 58,883 |
| KRF-322 | 5.9 | 70,153 |
| KRF-323 | 5.9 | 32,933 |
| KRF-324 | 6.1 | 56,989 |
| KRF-325 | 5.4 | 24,595 |
| KRF-326 | 5.6 | 15,368 |
| KRF-327 | 5.9 | 47,074 |
| KRF-328 | 5.9 | 22,165 |
| KRF-329 | 5.7 | 100,420 |
| KRF-330 | 5.1 | 79,642 |
| KRF-331 | 7.1 | 47,142 |
| KRF-332 | 5.9 | 66,491 |
| KRF-333 | 5.8 | 67,137 |
| KRF-334 | 4.4 | 12,184 |
| KRF-335 | 5.9 | 95,725 |
| KRF-336 | 5.9 | 23,420 |
| KRF-337 | 5.8 | 97,397 |
| KRF-338 | 5.8 | 71,160 |
| KRF-339 | 6.4 | 44,084 |
| KRF-340 | 6.0 | 51,612 |
| KRF-341 | 5.8 | 48,456 |
| KRF-342 | 6.1 | 24,316 |
| KRF-343 | 7.8 | 46,948 |
| KRF-344 | 5.8 | 24,239 |
| KRF-345 | 5.6 | 91,497 |
| KRF-346 | 5.8 | 58,085 |
| KRF-347 | 4.6 | 67,652 |
| KRF-348 | 4.8 | 115,177 |
| KRF-349 | 5.3 | 49,677 |
| KRF-350 | 8.3 | 63,976 |
| KRF-351 | 8.5 | 49,211 |
| KRF-352 | 7.8 | 66,706 |

For any given KRF, the signal obtained upon analyzing a sample (e.g., blood or kidney tissue) from subjects having kidney response relative to the signal obtained upon analyzing a sample (e.g., blood or kidney tissue) from subjects free from kidney response will depend upon the particular analytical protocol and detection technique that is used. Accordingly, the present invention contemplates that each laboratory will, based on the present description, establish a reference range for each KRF in subjects free from kidney response according to the analytical protocol and detection technique in use, as is conventional in the diagnostic art. Preferably, at least one control positive sample (e.g., blood or kidney tissue) from a subject known to have kidney response or at least one control negative sample (e.g., blood or kidney tissue) from a subject known to be free from kidney response (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no discernable protein feature.

In a preferred embodiment, the signal associated with a KRF in the kidney tissue of a subject (e.g., a subject suspected of having or known to have kidney response) is normalized with reference to one or more ERFs detected in the same 2D gel. As will be apparent to one of ordinary skill in the art, such ERFs may readily be determined by comparing different samples using the Preferred Technology. Suitable ERFs include (but are not limited to) that described in the following table.

TABLE V
Expression Reference Features in Kidney Tissue

| ERF | pI | MW (Da) |
|---|---|---|
| ERF-1 | 5.32 | 20135 |
| ERF-2 | 4.61 | 28930 |

In another preferred embodiment, the signal associated with a KRF in the blood of a subject (e.g., a subject suspected of having or known to have kidney response) is normalized with reference to one or more ERFs detected in the same 2D gel. As will be apparent to one of ordinary skill in the art, such ERFs may readily be determined by comparing different samples using the Preferred Technology. Suitable ERFs include (but are not limited to) that described in the following table.

TABLE VI
Expression Reference Features in Blood

| ERF | pI | MW (Da) |
|---|---|---|
| ERF-3 | 5.5 | 36283 |
| ERF-4 | 5.31 | 36739 |
| ERF-5 | 5.47 | 70662 |

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight and the apparent isoelectric point of a feature or protein isoform as measured in exact accordance with the Reference Protocol identified in Section 6 below. When the Reference Protocol is followed and when samples are run in duplicate or a higher number of replicates, variation in the measured mean pI of a KRF or KRPI is typically less than 3% and variation in the measured mean MW of a KRFor KRPI is typically less than 5%. Where the skilled artisan wishes to deviate from the Reference Protocol, calibration experiments should be performed to compare the MW and pI for each KRF or protein isoform as detected (a) by the Reference Protocol and (b) by the deviant protocol.

KRFs can be used for detection, prognosis, diagnosis, or monitoring of kidney response, or for identifying patients most likely to respond to a specific therapeutic treatment, or for drug development. In one embodiment of the invention, kidney tissue from a subject (e.g., a subject treated with a drug candidate or suspected of having kidney response) is analyzed by 2D electrophoresis for quantitative detection of one or more of the following KRFs: KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-105, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180. A decreased abundance of said one or more KRFs in the kidney tissue from the subject relative to kidney tissue from a subject or subjects free from kidney response (e.g., a control sample or a previously determined reference range) indicates the presence of kidney response.

In another embodiment of the invention, kidney tissue from a subject is analyzed by 2D electrophoresis for quantitative detection of one or more of the following KRFs: KRF-8, KRF-9, KRF-22, KRF-27, KRF-28, KRF-30, KRF-36, KRF-38, KRF-47, KRF-51, KRF-54, KRF-67, KRF-68, KRF-97, KRF-111, KRF-112, KRF-116, KRF-140, KRF-141, KRF-142, KRF-144, KRF-145, KRF-147, KRF-148, KRF-149, KRF-150, KRF-151, KRF-158, KRF-162, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288, KRF-289. An increased abundance of said one or more KRFs in the kidney tissue from the subject relative to kidney tissue from a subject or subjects free from kidney response (e.g., a control sample or a previously determined reference range) indicates the presence of kidney response.

In yet another embodiment, kidney tissue from a subject is analyzed by 2D electrophoresis for quantitative detection of (a) one or more KRFs or any combination of them, whose decreased abundance indicates the presence of kidney response, i.e., KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-105, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180; and (b) one or more KRFs or any combination of them, whose increased abundance indicates the presence of kidney response i.e., KRF-8, KRF-9, KRF-22, KRF-27, KRF-28, KRF-30, KRF-36, KRF-38, KRF-47, KRF-51, KRF-54, KRF-67, KRF-68, KRF-97, KRF-111, KRF-112, KRF-116, KRF-140, KRF-141, KRF-142, KRF-144, KRF-145, KRF-147, KRF-148, KRF-149, KRF-150, KRF-151, KRF-158, KRF-162, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288, KRF-289.

In yet another embodiment of the invention, kidney tissue from a subject is analyzed by 2D electrophoresis for quantitative detection of one or more of the following KRFs: KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-15, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288, KRF-289 wherein the ratio of the one or more KRFs relative to an Expression Reference Feature (ERF) indicates whether kidney response is present. In a specific embodiment, a decrease in one or more KRF/ERF ratios in a test sample relative to the KRF/ERF ratios in a control sample or a reference range indicates the presence of kidney response; KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-105, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180 are suitable KRFs for this purpose. In another specific embodiment, an increase in one or more KRF/ERF ratios in a test sample relative to the KRF/ERF ratios in a control sample or a reference range indicates the presence of kidney response; KRF-8, KRF-9, KRF-22, KRF-27, KRF-28, KRF-30, KRF-36, KRF-38, KRF-47, KRF-51, KRF-54, KRF-67, KRF-68, KRF-97, KRF-111, KRF-112, KRF-116, KRF-140, KRF-141, KRF-142, KRF-144, KRF-145, KRF-147, KRF-148, KRF-149, KRF-150, KRF-151, KRF-158, KRF-162, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288, KRF-289 are suitable KRFs for this purpose.

In a further embodiment of the invention, kidney tissue from a subject is analyzed by 2D electrophoresis for quantitative detection of (a) one or more KRFs, or any combination of them, whose decreased KRF/ERF ratio(s) in a test sample relative to the KRF/ERF ratio(s) in a control sample indicates the presence of kidney response, i.e., KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-105, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180; (b) one or more KRFs, or any combination of them, whose increased KRF/ERF ratio(s) in a test sample relative to the KRF/ERF ratio(s) in a control sample indicates the presence of kidney response, i.e., KRF-8, KRF-9, KRF-22, KRF-27, KRF-28, KRF-30, KRF-36, KRF-38, KRF-47, KRF-51, KRF-54, KRF-67, KRF-68, KRF-97, KRF-111, KRF-112, KRF-116, KRF-140, KRF-141, KRF-142, KRF-144, KRF-145, KRF-147, KRF-148, KRF-149, KRF-150, KRF-151, KRF-158, KRF-162, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288, KRF-289.

In a preferred embodiment, kidney tissue from a subject is analyzed for quantitative detection of a plurality of KRFs.

In another embodiment of the invention, blood from a subject (e.g., a subject suspected of having kidney response) is analyzed by 2D electrophoresis for quantitative detection of one or more of the following KRFs: KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313. A decreased abundance of said one or more KRFs in the blood from the subject relative to blood from a subject or subjects free from kidney response (e.g., a control sample or a previously determined reference range) indicates the presence of kidney response.

In another embodiment of the invention, blood from a subject is analyzed by 2D electrophoresis for quantitative detection of one or more of the following KRFs: KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351, KRF-352. An increased abundance of said one or more KRFs in the blood from the subject relative to blood from a subject or subjects free from kidney response (e.g., a control sample or a previously determined reference range) indicates the presence of kidney response.

In yet another embodiment, blood from a subject is analyzed by 2D electrophoresis for quantitative detection of (a) one or more KRFs or any combination of them, whose decreased abundance indicates the presence of kidney response, i.e., KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313; and (b) one or more KRFs or any combination of them, whose increased abundance indicates the presence of kidney response i.e., KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351, KRF-352.

In yet another embodiment of the invention, blood from a subject is analyzed by 2D electrophoresis for quantitative detection of one or more of the following KRFs: KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313, KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351, KRF-352 wherein the ratio of the one or more KRFs relative to an Expression Reference Feature (ERF) indicates whether kidney response is present. In a specific embodiment, a decrease in one or more KRF/ERF ratios in a test sample relative to the KRF/ERF ratios in a control sample or a reference range indicates the presence of kidney response; KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313 are suitable KRFs for this purpose.

In another specific embodiment, an increase in one or more KRF/ERF ratios in a test sample relative to the KRF/ERF ratios in a control sample or a reference range indicates the presence of kidney response; KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351, KRF-352 are suitable KRFs for this purpose.

In a further embodiment of the invention, blood from a subject is analyzed by 2D electrophoresis for quantitative detection of (a) one or more KRFs, or any combination of them, whose decreased KRF/ERF ratio(s) in a test sample relative to the KRF/ERF ratio(s) in a control sample indicates the presence of kidney response, i.e., KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313; (b) one or more KRFs, or any combination of them, whose increased KRF/ERF ratio(s) in a test sample relative to the KRF/ERF ratio(s) in a control sample indicates the presence of kidney response, i.e., KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351, KRF-352.

In a preferred embodiment, blood from a subject is analyzed for quantitative detection of a plurality of KRFs.

Kidney Response-Associated Protein Isoforms (KRPIs)

In another aspect of the invention, blood or kidney tissue from a subject, preferably a living subject, is analyzed for quantitative detection of one or more Kidney Response-Associated Protein Isoforms (KRPIs) for screening or diagnosis of kidney response, to determine the prognosis of a subject having kidney response, to monitor the effectiveness of kidney response therapy, for identifying patients most likely to respond to a particular therapeutic treatment or for drug development and, in particular, to determine the potential for drug candidates to induce a kidney response. As is well known in the art, a given protein may be expressed as one or more variants that differ in their amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pI, MW, or both. It follows that differential presence of a protein variant does not require differential expression of the gene encoding the protein in question. As used herein, the term “Kidney Response-Associated Protein Isoform” refers to a protein that is differentially present in a sample of blood or kidney tissue from a subject having kidney response compared with sample of blood or kidney tissue from a subject free from kidney response.

Four groups of KRPIs have been identified by amino acid sequencing of KRFs. KRPIs were isolated, subjected to proteolysis, and analyzed by mass spectrometry using the methods and apparatus of the Preferred Technology. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.ch/, and the European Molecular Biology Laboratory web site at www.mann.embl-heidelberg.de/Services/PeptideSearch/. Identification of KRPIs was performed primarily using the SEQUEST search program (Eng et al., 1994, J. Am. Soc. Mass Spectrom. 5:976-989) and the method described in PCT Application No. PCT/GB01/04034, which is incorporated herein by reference in its entirety.

The first group consists of KRPIs that are decreased in the kidney tissue of subjects having kidney response as compared with the kidney tissue of subjects free from kidney response, where the differential presence is significant. The amino acid sequences of tryptic digest peptides of these KRPIs identified by tandem mass spectrometry and database searching as described in the Examples, infra are listed in Table VII in addition to the pIs and MWs of these KRPIs.

TABLE VII

KRPIs Decreased in Kidney Tissue of Subjects
Having Kidney Response

| Table | | | | Amino Acid Sequences of | |
|---|---|---|---|---|---|
| VII KRF | KRPI | pI | MW (Da) | Tryptic Digest Peptides | Seq ID |
| KRF-2 | KRPI-2 | 7.3 | 35,621 | ALGLSNFSSR | SEQ ID 9 |
| | | | | SPAQILLR | SEQ ID 199 |
| | | | | YALSVGYR | SEQ ID 251 |
| KRF-8 | KRPI-8 | 5.0 | 24,350 | SIQEIQELDK | SEQ ID 196 |
| | | | | TDYMVGSYGPR | SEQ ID 213 |
| KRF-11 | KRPI-11 | 5.4 | 41,863 | DFDPAINEYIQR | SEQ ID 35 |
| | | | | FPPDNSAPYGAR | SEQ ID 78 |
| | | | | SRPSLPLPQSR | SEQ ID 201 |
| KRF-13 | KRPI-13 | 5.4 | 21,765 | DDNPNLPPFQR | SEQ ID 34 |
| | | | | DNYGELADCCAK | SEQ ID 40 |
| | | | | LVQEVTDFAK | SEQ ID 153 |
| | | | | TCVADENAENCDK | SEQ ID 212 |
| | | | | TNEVLTQCCTESDK | SEQ ID 261 |
| KRF-14 | KRPI-14 | 6.8 | 12,639 | HVPGASFFDIEECR | SEQ ID 110 |
| | | | | TVSVLNGGFR | SEQ ID 230 |
| | | | | VLDASWYSPGTR | SEQ ID 237 |
| KRF-15 | KRPI-15 | 5.0 | 25,902 | LFIVGSNSSSSTR | SEQ ID 136 |
| | | | | QFDIQLLTHNDPK | SEQ ID 179 |
| | | | | TLNEWSSQISPDLVR | SEQ ID 223 |
| KRF-16 | KRPI-16 | 5.2 | 21,913 | SLHTLFGDK | SEQ ID 197 |
| KRF-19 | KRPI-19 | 7.0 | 21,399 | EESLALAVK | SEQ ID 48 |
| | | | | KPPPDGHYVDVVR | SEQ ID 125 |
| | | | | LYYFQGR | SEQ ID 155 |
| | | | | YFPVFEK | SEQ ID 254 |
| KRF-21 | KRPI-21 | 5.4 | 80,627 | LASDLLEWIR | SEQ ID 130 |
| | | | | TINEVENQILTR | SEQ ID 221 |
| KRF-23 | KRPI-23 | 7.2 | 20,698 | ILGADTSVDLEETGR | SEQ ID 116 |
| | | | | VLSIGDGIAR | SEQ ID 238 |
| KRF-27 | KRPI-27 | 4.9 | 31,623 | GLGTDEDSILNLLTAR | SEQ ID 94 |
| | | | | GTVTDFSGFDGR | SEQ ID 99 |
| | | | | VLTEIIASR | SEQ ID 239 |
| KRF-28 | KRPI-28 | 5.6 | 42,298 | SENEPIENEAAR | SEQ ID 193 |
| | | | | INFDDNAEFR | SEQ ID 117 |
| KRF-35 | KRPI-35 | 5.7 | 34,167 | GNLTDLETNGVR | SEQ ID 97 |
| | | | | MPINEPAPGR | SEQ ID 160 |
| | | | | TEDIITTI | SEQ ID 214 |
| KRF-40 | KRPI-40 | 5.3 | 80,900 | TINEVENQILTR | SEQ ID 221 |
| KRF-41 | KRPI-41 | 5.8 | 43,502 | APQVSTPTLVEAAR | SEQ ID 12 |
| | | | | ECCHGDLLECADDR | SEQ ID 46 |
| | | | | FPNAEFAEITK | SEQ ID 77 |
| | | | | LPCVEDYLSAILNR | SEQ ID 145 |
| KRF-42 | KRPI-42 | 5.8 | 39,836 | IGAEVYHNLK | SEQ ID 113 |
| | | | | VNQIGSVTESLQACK SEQ ID 244 | |
| | | | | YITPDQLADLYK | SEQ ID 256 |
| KRF-43 | KRPI-43 | 6.8 | 21,939 | FVEGLPINDFSR | SEQ ID 81 |
| | | | | GEFITTVQQR | SEQ ID 88 |
| KRF-45 | KRPI-45.1 | 7.1 | 23,849 | GAVHQLCQSLAGK | SEQ ID 85 |
| | | | | RPNSGSLIQVVTTDGK | SEQ ID 188 |
| KRF-45 | KRPI-45.2 | 7.1 | 23,849 | YNLGLDLR | SEQ ID 262 |
| | | | | HGGTIPVVPTAEFQDR | SEQ ID 105 |
| KRF-57 | KRPI-57 | 6.8 | 11,462 | EVLDILTAELHR | SEQ ID 65 |
| | | | | IEVYMDGGVR | SEQ ID 111 |
| | | | | QLDEVSASIDALR | SEQ ID 182 |
| KRF-59 | KRPI-59 | 5.7 | 27,218 | TQAMGLWAQPR | SEQ ID 226 |
| | | | | GNDISSGTVLSEYVGSGP | |
| KRF-60 | KRPI-60 | 5.3 | 20,135 | PK | SEQ ID 95 |
| | | | | LYTLVLTDPDAPSR | SEQ ID 154 |
| | | | | VDYGGVTVDELGK | SEQ ID 233 |
| | | | | VLTPTQVMNR | SEQ ID 240 |
| KRF-63 | KRPI-63 | 6.4 | 32,486 | ARPFPDGLAEDIDK | SEQ ID 14 |
| | | | | GGGQIIPTAR | SEQ ID 89 |
| | | | | YEWDVAEAR | SEQ ID 253 |
| KRF-70 | KRPI-70 | 7.7 | 20,347 | AVAFQNPQTR | SEQ ID 19 |
| | | | | ENMAYTVEGIR | SEQ ID 58 |
| | | | | EVAEQFLNIR | SEQ ID 62 |
| | | | | NALANPLYCPDYR | SEQ ID 164 |
| | | | | RWEVAALR | SEQ ID 189 |
| KRF-72 | KRPI-72 | 7.6 | 37,026 | AGLSNFSSR | SEQ ID 9 |
| | | | | DAGHPLYPFNDPY | SEQ ID 28 |
| | | | | GLEVTAYSPLGSSDR | SEQ ID 93 |
| | | | | HHPEDVEPAVR | SEQ ID 106 |
| | | | | MPLIGLGTWK | SEQ ID 162 |
| | | | | QIDDVLSVASVR | SEQ ID 181 |
| | | | | YALSVGYR | SEQ ID 251 |
| KRF-73 | KRPI-73 | 7.3 | 27,831 | AFPAWADTSILSR | SEQ ID 5 |
| | | | | DMDLYSYR | SEQ ID 38 |
| | | | | VPGATMLLAK | SEQ ID 246 |
| | | | | WIDIHNPATNEVVGR | SEQ ID 249 |
| KRF-76 | KRPI-76 | 7.0 | 24,556 | DDGSWEVIEGYR | SEQ ID 32 |
| | | | | MVEGFFDR | SEQ ID 163 |
| KRF-84 | KRPI-84 | 6.3 | 21,397 | AVDSLVPIGR | SEQ ID 20 |
| | | | | ILGADTSVDLEETGR | SEQ ID 116 |
| | | | | NVQAEEMVEFSSGLK SEQ ID 177 | |
| | | | | TGAIVDVPVGDELLGR | SEQ ID 218 |
| | | | | TGTAEMSSILEER | SEQ ID 220 |
| | | | | VLSIGDGIAR | SEQ ID 238 |
| KRF-85 | KRPI-85 | 7.3 | 18,969 | DMDLYSYR | SEQ ID 38 |
| | | | | LITLEQGK | SEQ ID 140 |
| KRF-86 | KRPI-86 | 5.6 | 11,175 | EVLDILTAELHR | SEQ ID 65 |
| | | | | IEVYMDGGVR | SEQ ID 111 |
| | | | | QLDEVSASIDALR | SEQ ID 182 |
| KRF-88 | KRPI-88 | 7.7 | 18,953 | ALGLSNFSSR | SEQ ID 9 |
| | | | | GLEVTAYSPLGSSDR | SEQ ID 93 |
| | | | | QIDDVLSVASVR | SEQ ID 181 |
| KRF-90 | KRPI-90 | 8.5 | 16,508 | ASAELALGENSEVLK | SEQ ID 15 |
| | | | | DAGMQLQGYR | SEQ ID 29 |
| | | | | DDNGKPYVLPSVR | SEQ ID 33 |
| | | | | FVTVQTISGTGALR | SEQ ID 82 |
| | | | | MNLGVGAYR | SEQ ID 159 |
| KRF-91 | KRPI-91 | 6.0 | 13,898 | ELY LVAYK | SEQ ID 56 |
| | | | | LCEAHGITR | SEQ ID 132 |
| KRF-98 | KRPI-98 | 6.1 | 20,618 | ANVDKPGLVDDFK | SEQ ID 10 |
| | | | | SWNETFHTR | SEQ ID 206 |
| | | | | YTALVDAEEK | SEQ ID 264 |
| KRF-101 | KRPI-101 | 7.7 | 24,269 | APQVSTPTLVEAAR | SEQ ID 12 |
| | | | | CCTLPEAQR | SEQ ID 25 |
| | | | | KYEATLEK | SEQ ID 127 |
| | | | | LPCVEDYLSAILNR | SEQ ID 145 |
| | | | | QTALAELVK | SEQ ID 184 |
| | | | | TNCELYEK | SEQ ID 224 |
| KRF-104 | KRPI-104 | 7.1 | 26,948 | CAVVDVPFGGAK | SEQ ID 23 |
| | | | | DDGSWEVIEGYR | SEQ ID 32 |
| KRF-105 | KRPI-105 | 9.4 | 34,066 | IGGIGTVPVGR | SEQ ID 115 |
| | | | | LPLQDVYK | SEQ ID 146 |
| | | | | RYEEIVK | SEQ ID 190 |
| KRF-113 | KRPI-113 | 7.1 | 35,304 | SYLSWLTER | SEQ ID 209 |
| | | | | VASFEEVVR | SEQ ID 232 |
| KRF-122 | KRPI-122 | 5.3 | 11,763 | EIMIAAQR | SEQ ID 51 |
| | | | | GLDPYNMLPPK | SEQ ID 92 |
| KRF-123 | KRPI-123 | 5.0 | 44,701 | SSEEIESAFR | SEQ ID 202 |
| KRF-128 | KRPI-128 | 7.5 | 28,930 | DTPGFIVNR | SEQ ID 43 |
| | | | | TFESLVDFCK | SEQ ID 217 |
| KRF-131 | KRPI-131 | 7.9 | 37,143 | DAGTIAGLNVLR | SEQ ID 30 |
| | | | | FEELNADLFR | SEQ ID 68 |
| | | | | RFDDAVVQSDMK | SEQ ID 185 |
| | | | | SQIHDIVLVGGSTR | SEQ ID 200 |
| | | | | STAGDTHLGGEDFDNR | SEQ ID 203 |
| | | | | TTPSYVAFTDTER | SEQ ID 227 |
| KRF-132 | KRPI-132 | 7.0 | 36,051 | HGGTIPVVPTAEFQDR | SEQ ID 104 |
| | | | | LQHGSILGFPK | SEQ ID 148 |
| | | | | YNLGLDLR | SEQ ID 262 |
| KRF-134 | KRPI-134 | 5.6 | 24,011 | TPAQFDADELR | SEQ ID 225 |
| KRF-138 | KRPI-138 | 7.6 | 35,652 | INISEGNCPER | SEQ ID 118 |
| KRF-139 | KRPI-139 | 7.1 | 27,742 | ALEESNYELEGK | SEQ ID 8 |
| KRF-142 | KRPI-142 | 6.0 | 78,163 | APDFVFYAPR | SEQ ID 11 |
| | | | | IGFPWSEIR | SEQ ID 114 |
| | | | | QLLTLSNELSQAR | SEQ ID 183 |
| KRF-143 | KRPI-143 | 7.7 | 26,909 | HTTIFEVLPQK | SEQ ID 109 |
| | | | | KPVDQYEDCYLAR | SEQ ID 126 |
| | | | | TVLPADGPR | SEQ ID 229 |
| KRF-144 | KRPI-144 | 6.8 | 23,369 | GIMGEDSYPYIGK | SEQ ID 90 |
| KRF-149 | KRPI-149 | 7.4 | 51,414 | DDGSWEVIEGYR | SEQ ID 32 |
| | | | | HGGTIPVVPTAEFQDR | SEQ ID 104 |
| | | | | MVEGFFDR | SEQ ID 163 |
| | | | | YNLGLDLR | SEQ ID 262 |
| KRF-152 | KRPI-152 | 5.2 | 57,842 | AGFAGDDAPR | SEQ ID 6 |
| | | | | AVFPSIVGR | SEQ ID 21 |
| | | | | GYSFTTTAER | SEQ ID 100 |
| | | | | IWHHTFYNELR | SEQ ID 124 |
| | | | | QEYDESGPSIVHR | SEQ ID 178 |
| | | | | SYELPDGQVITIGNER | SEQ ID 208 |
| | | | | VAPEEHPVLLTEAPLNPK | SEQ ID 231 |
| KRF-153 | KRPI-153 | 5.7 | 55,401 | NPSVLLTLR | SEQ ID 173 |
| | | | | YCTDTSIIFR | SEQ ID 252 |
| KRF-158 | KRPI-158 | 5.5 | 67,349 | APQVSTPTLVEAAR | SEQ ID 13 |
| KRF-159 | KRPI-159 | 6.9 | 40,414 | ASSTANLIFEDCR | SEQ ID 16 |
| | | | | EHLFPTSQVK | SEQ ID 50 |
| | | | | HAFGAPLTK | SEQ ID 101 |
| | | | | ITEIYEGTSEIQR | SEQ ID 123 |
| | | | | LADMALALESAR | SEQ ID 128 |
| KRF-168 | KRPI-168 | 8.9 | 24,933 | ENFSCLTR | SEQ ID 57 |
| | | | | EVGVYEALKDDSWLK | SEQ ID 64 |
| | | | | FVEGLPINDFSR | SEQ ID 81 |
| | | | | GEFITTVQQR | SEQ ID 88 |
| | | | | LGVTADDVK | SEQ ID 137 |
| | | | | VIVVGNPANTNCLTASK | SEQ ID 236 |
| KRF-170 | KRPI-170 | 8.8 | 26,806 | LTFDSSFSPNTGK | SEQ ID 150 |
| | | | | VTQSNFAVGYK | SEQ ID 247 |
| | | | | YQVDPDACFSAK | SEQ ID 263 |
| KRF-178 | KRPI-178 | 6.6 | 22,591 | CNVSEGVAQCTR | SEQ ID 27 |
| | | | | DLGATWVVLGHSER | SEQ ID 37 |
| | | | | TATPQQAQEVHEK | SEQ ID 210 |
| KRF-179 | KRPI-179 | 8.7 | 27,848 | AVDSLVPIGR | SEQ ID 20 |
| | | | | TGAIVDVPVGDELLGR | SEQ ID 218 |
| KRF-139 | KRPI-285 | 7.1 | 27,742 | FAELAQIYAR | SEQ ID 66 |
| | | | | VGLGICYDMR | SEQ ID 235 |
| KRF-139 | KRPI-286 | 7.1 | 27,742 | EVLDILTAELHR | SEQ ID 65 |
| | | | | QLDEVSASIDALR | SEQ ID 182 |

The second group comprises KRPIs that are increased in the kidney tissue of subjects having kidney response as compared with the kidney tissue of subjects free from kidney response, where the differential presence is significant. The amino acid sequences of tryptic digest peptides of these KRPIs identified by tandem mass spectrometry and database searching are listed in Table VIII in addition to the pIs and MWs of these KRPIs.

TABLE VIII

KRPIs Increased in Kidney Tissue of Subjects Having Kidney Response

| Table | | | | Amino Acid Sequences of | |
|---|---|---|---|---|---|
| VIII KRF | KRPI | pI | MW (Da) | Tryptic Digest Peptides | Seq ID |
| KRF-8 | KRPI-8 | 5.0 | 24,350 | SIQEIQELDK | SEQ ID 196 |
| | | | | TDYMVGSYGPR | SEQ ID 213 |
| KRF-27 | KRPI-27 | 4.9 | 31,623 | GLGTDEDSILNLLTAR | SEQ ID 94 |
| | | | | GTVTDFSGFDGR | SEQ ID 99 |
| | | | | VLTEIIASR | SEQ ID 239 |
| KRF-28 | KRPI-28 | 5.6 | 42,298 | SENEPIENEAAR | SEQ ID 193 |
| | | | | INFDDNAEFR | SEQ ID 117 |
| KRF-140 | KRPI-140 | 7.1 | 34,055 | NHFTVAQNER | SEQ ID 168 |
| KRF-142 | KRPI-142 | 6.0 | 78,163 | APDFVFYAPR | SEQ ID 11 |
| | | | | IGFPWSEIR | SEQ ID 114 |
| | | | | QLLTLSNELSQAR | SEQ ID 183 |
| KRF-144 | KRPI-144 | 6.8 | 23,369 | GIMGEDSYPYIGK | SEQ ID 90 |
| KRF-149 | KRPI-149 | 7.4 | 51,414 | DDGSWEVIEGYR | SEQ ID 32 |
| | | | | HGGTIPVVPTAEFQDR | SEQ ID 104 |
| | | | | MVEGFFDR | SEQ ID 163 |
| | | | | YNLGLDLR | SEQ ID 262 |
| KRF-158 | KRPI-158 | 5.5 | 67,349 | APQVSTPTLVEAAR | SEQ ID 13 |
| KRF-183 | KRPI-183 | 7.9 | 34,066 | ELADIAHR | SEQ ID 54 |
| | | | | LQSIGTENTEENR | SEQ ID 149 |
| KRF-184 | KRPI-184 | 6.2 | 45,875 | LPSDVVTAVR | SEQ ID 147 |
| | | | | TEQGPPSSEYIFER | SEQ ID 216 |
| KRF-185 | KRPI-185 | 5.7 | 44,444 | EQIDIFEGIK | SEQ ID 60 |
| | | | | ETYLAILMDR | SEQ ID 61 |
| | | | | INFDDNAEFR | SEQ ID 117 |
| | | | | LEGTNVQEAQNILK | SEQ ID 135 |
| | | | | MAENLGFLGSLK | SEQ ID 156 |
| | | | | SENEPIENEAAR | SEQ ID 193 |
| | | | | VMVAEALDISR | SEQ ID 243 |
| KRF-186 | KRPI-186 | 6.2 | 35,095 | DLDVAVLVGSMPR | SEQ ID 36 |
| | | | | ENFSCLTR | SEQ ID 57 |
| | | | | FVEGLPINDFSR | SEQ ID 81 |
| | | | | GEFITTVQQR | SEQ ID 88 |
| KRF-188 | KRPI-188 | 6.3 | 42,667 | RPEFQALR | SEQ ID 186 |
| | | | | SVSLQYLEAVR | SEQ ID 204 |
| KRF-189 | KRPI-189.1 | 7.5 | 37,358 | ALGLSNFSSR | SEQ ID 9 |
| | | | | DAGHPLYPFNDPY | SEQ ID 28 |
| | | | | GLEVTAYSPLGSSDR | SEQ ID 93 |
| | | | | HHPEDVEPAVR | SEQ ID 106 |
| | | | | MPLIGLGTWK | SEQ ID 162 |
| | | | | QIDDVLSVASVR | SEQ ID 181 |
| | | | | YALSVGYR | SEQ ID 251 |
| | | | | YIVPMITVDGK | SEQ ID 257 |
| KRF-189 | KRPI-189.2 | 7.5 | 37,358 | MGLALISGYNLFR | SEQ ID 158 |
| | | | | YHPAQPLHMK | SEQ ID 255 |
| | | | | GQIIQVEAPWIK, | SEQ ID 98 |
| KRF-192 | KRPI-192 | 6.8 | 22,439 | VTQSNFAVGYK | SEQ ID 247 |
| | | | | WTEYGLTFTEK | SEQ ID 250 |
| KRF-196 | KRPI-196 | 4.7 | 26,603 | DSTLIMQLLR | SEQ ID 42 |
| | | | | EKIEMELR | SEQ ID 53 |
| | | | | FLIPNASQPESK | SEQ ID 72 |
| | | | | NLLSVAYK | SEQ ID 169 |
| | | | | SVTEQGAELSNEER | SEQ ID 205 |
| | | | | YLAEVAAGDDKK | SEQ ID 258 |
| KRF-202 | KRPI-202 | 5.5 | 31,104 | EPPFPLSTR | SEQ ID 59 |
| | | | | FANTMGLVIER | SEQ ID 67 |
| | | | | QGEIFLLPAR | SEQ ID 180 |
| | | | | SWVEENR | SEQ ID 207 |
| | | | | TGKPNPDQLLK | SEQ ID 219 |
| KRF-203 | KRPI-203 | 7.5 | 30,601 | VLVAQHDAYK | SEQ ID 241 |
| KRF-206 | KRPI-206 | 7.6 | 54,603 | DMDLYSYR | SEQ ID 38 |
| | | | | EGASILLDGR | SEQ ID 49 |
| | | | | TLADAEGDVFR | SEQ ID 222 |
| KRF-208 | KRPI-208 | 4.8 | 15,906 | NGQGSDPAVTYYR | SEQ ID 167 |
| KRF-210 | KRPI-210 | 8.0 | 35,549 | LISWYDNEYGYSNR | SEQ ID 139 |
| KRF-219 | KRPI-219 | 7.4 | 13,463 | GDFCIQVGR | SEQ ID 86 |
| | | | | LVAMKFLR | SEQ ID 151 |
| | | | | SCAHDWVYE | SEQ ID 191 |
| | | | | VMLGETNPADSKPGT | SEQ ID 242 |
| | | | | IR | |
| KRF-222 | KRPI-222 | 7.5 | 57,015 | EDGGGWWYNR | SEQ ID 47 |
| | | | | TENGGWTVIQNR | SEQ ID 215 |
| KRF-225 | KRPI-225 | 6.0 | 50,369 | NPDSLELIR | SEQ ID 172 |
| KRF-229 | KRPI-229 | 7.5 | 22,173 | DPQHDLER | SEQ ID 41 |
| | | | | VPDFSDYR | SEQ ID 245 |
| KRF-232 | KRPI-232 | 7.1 | 40,809 | AIDVGQGQTR | SEQ ID 7 |
| | | | | ASSVVVSGTPIR | SEQ ID 17 |
| | | | | FGEPIPISK | SEQ ID 70 |
| | | | | HLFTGPVLSK | SEQ ID 107 |
| KRF-234 | KRPI-234 | 4.9 | 18,919 | EVAGFWVK | SEQ ID 63 |
| KRF-235 | KRPI-235.1 | 7.1 | 43,682 | ATDFVVPGPG | K SEQ ID 18 |
| | | | | TVEAEAAHGTVTR | SEQ ID 228 |
| KRF-235 | KRPI-235.2 | 7.1 | 43,682 | HFMAPGVR | SEQ ID 102 |
| | | | | SCWDEPLSITVR | SEQ ID 192 |
| | | | | IEYFEEAVNYLR | SEQ ID 112 |
| | | | | ADAGGELDLAR | SEQ ID 2 |
| | | | | GLAPEQPVTLR, | SEQ ID 91 |
| KRF-236 | KRPI-236 | 5.5 | 13,445 | EKIEENGSMR | SEQ ID 52 |
| | | | | ELYLVAYK | SEQ ID 56 |
| | | | | LCEAHGITR | SEQ ID 132 |
| | | | | LNGDWFSIVVASNK | SEQ ID 143 |
| | | | | NGETFQLMVLYGR | SEQ ID 166 |
| | | | | YVMFHLINFK, | SEQ ID 266 |
| KRF-237 | KRPI-237 | 9.1 | 23,172 | FIQSPEDLEK | SEQ ID 71 |
| | | | | SHGQDYLVGNR | SEQ ID 194 |
| KRF-240 | KRPI-240 | 7.5 | 22,637 | FNVWDTAGQEK | SEQ ID 75 |
| | | | | HLTGEFEK | SEQ ID 108 |
| | | | | NLQYYDISAK | SEQ ID 171 |
| | | | | NVPNWHR | SEQ ID 176 |
| | | | | SNYNFEKPFLWLAR | SEQ ID 198 |
| KRF-245 | KRPI-245 | 4.7 | 43,086 | FTPGTFTNQIQAAFR | SEQ ID 80 |
| | | | | LLVVTDPR | SEQ ID 141 |
| KRF-247 | KRPI-247 | 7.3 | 59,544 | DAQLFIQR | SEQ ID 31 |
| | | | | FSTVAGESGSADTVR | SEQ ID 79 |
| | | | | LAQEDPDYGLR | SEQ ID 129 |
| | | | | LNIMTAGPR | SEQ ID 144 |
| | | | | NLPVEEAGR | SEQ ID 170 |
| KRF-249 | KRPI-249 | 5.4 | 70,401 | DAGTIAGLNVLR | SEQ ID 30 |
| | | | | FEELNADLFR | SEQ ID 68 |
| | | | | FELTGIPPAPR | SEQ ID 69 |
| | | | | NQVAMNPTNTVFDA | SEQ ID 175 |
| | | | | K | |
| | | | | RFDDAVVQSDMK | SEQ ID 185 |
| | | | | SQIHDIVLVGGSTR | SEQ ID 200 |
| | | | | STAGDTHLGGEDFDN | SEQ ID 203 |
| | | | | R | |
| | | | | TTPSYVAFTDTER | SEQ ID 227 |
| | | | | VEIIANDQGNR | SEQ ID 234 |
| KRF-250 | KRPI-250 | 7.6 | 59,990 | FNSANEDNVTQVR | SEQ ID 74 |
| | | | | LAQEDPDYGLR | SEQ ID 129 |
| | | | | LNIMTAGPR | SEQ ID 144 |
| KRF-252 | KRPI-252 | 4.9 | 53,963 | AVLVDLEPGTMDSVR | SEQ ID 22 |
| | | | | FPGQLNADLR | SEQ ID 76 |
| | | | | ISEQFTAMFR | SEQ ID 122 |
| | | | | LAVNMVPFPR | SEQ ID 131 |
| | | | | LHFFMPGFAPLTSR | SEQ ID 138 |
| | | | | TAVCDIPPR | SEQ ID 211 |
| | | | | YLTVAAVFR | SEQ ID 260 |
| KRF-253 | KRPI-253 | 9.6 | 48,151 | IGGIGTVPVGR | SEQ ID 115 |
| | | | | LPLQDVYK | SEQ ID 146 |
| KRF-256 | KRPI-256 | 5.3 | 13,604 | DNIIDLTK | SEQ ID 39 |
| | | | | ELYLVAYK | SEQ ID 56 |
| | | | | LCEAHGITR | SEQ ID 132 |
| | | | | LNGDWFSIVVASNK | SEQ ID 143 |
| | | | | NGETFQAMVLYGR | SEQ ID 165 |
| | | | | YVMFHLINFK | SEQ ID 265 |
| KRF-257 | KRPI-257 | 4.7 | 12,867 | LCVAHGITR | SEQ ID 133 |
| | | | | LNGDWFSIVVASDK | SEQ ID 142 |
| KRF-263 | KRPI-263 | 7.7 | 42,563 | ELFAQEAFAPFR | SEQ ID 55 |
| | | | | AEVQTLVSR | SEQ ID 3 |
| KRF-266 | KRPI-266 | 5.5 | 23,258 | GATQQILDEAER | SEQ ID 84 |
| KRF-267 | KRPI-267 | 5.6 | 28,492 | GAEIVADTFR | SEQ ID 83 |
| | | | | RPEVDGVR | SEQ ID 187 |
| KRF-273 | KRPI-273 | 4.7 | 31,342 | IQLVEEELDR | SEQ ID 120 |
| | | | | IQVLQQQADDAEER | SEQ ID 121 |
| | | | | LVIIEGDLER | SEQ ID 152 |
| KRF-278 | KRPI-278 | 7.2 | 78,547 | IPSHAVVAR | SEQ ID 119 |
| | | | | TVLPADGPR | SEQ ID 229 |
| KRF-280 | KRPI-280 | 6.5 | 43,914 | AFEEEQALR | SEQ ID 4 |
| | | | | MGFEPLAYK | SEQ ID 157 |
| | | | | MPINEPAPGR | SEQ ID 160 |
| | | | | TEDIITTIR | SEQ ID 161 |
| KRF-282 | KRPI-282 | 4.7 | 15,768 | EAFNMIDQNR | SEQ ID 45 |
| | | | | GNFNYIEFTR | SEQ ID 96 |

The third group consists of KRPIs that are decreased in the blood of subjects having kidney response as compared with the blood of subjects free from kidney response, where the differential presence is significant. The amino acid sequences of tryptic digest peptides of these KRPIs identified by tandem mass spectrometry and database searching as described in the Examples, infra are listed in Table IX in addition to the pIs and MWs of these KRPIs.

TABLE IX

KRPIs Decreased in the Blood of Subjects Having Kidney Response

| Table | | | | Amino Acid Sequences of | |
|---|---|---|---|---|---|
| IX KRF | KRPI | pI | MW(Da) | Tryptic Digest Peptides | Seq ID |
| KRF-313 | KRPI-313 | 4.9 | 53,018 | CNADPGLSALLSDHR | SEQ ID 26 |
| | | | | DYFISCPGR | SEQ ID 44 |
| | | | | FNPVTGEVPPR | SEQ ID 73 |
| | | | | GECQSEGVLFFQGNR | SEQ ID 87 |
| | | | | NPVTSVDAAFR | SEQ ID 174 |
| | | | | VWVYPPEK | SEQ ID 248 |

The fourth group consists of KRPIs that are increased in the blood of subjects having kidney response as compared with the blood of subjects free from kidney response, where the differential presence is significant. The amino acid sequences of tryptic digest peptides of these KRPIs identified by tandem mass spectrometry and database searching as described in the Examples, infra are listed in Table X in addition to the pIs and MWs of these KRPIs.

TABLE X

KRPIs increased in the Blood of Subjects Having Kidney Response

| Table X | | | | Amino Acid Sequences of | |
|---|---|---|---|---|---|
| KRF | KRPI | pI | MW (Da) | Tryptic Digest Peptides | Seq ID |
| KRF-314 | KRPI-314.1 | 5.7 | 35,921 | AADKDNCFATEGPNLVAR | SEQ ID 1 |
| | | | | APQVSTPTLVEAAR | SEQ ID 12 |
| | | | | CCSGSLVER | SEQ ID 24 |
| | | | | CCTLPEAQR | SEQ ID 25 |
| | | | | FPNAEFAEITK | SEQ ID 77 |
| | | | | LCVLHEK | SEQ ID 134 |
| | | | | SIHTLFGDK | SEQ ID 195 |
| KRF-314 | KRPI-314.2 | 5.7 | 35,921 | DYFISCPGR | SEQ ID 44 |
| | | | | FNPVTGEVPPR | SEQ ID 73 |
| | | | | GECQSEGVLFFQGNR | SEQ ID 87 |
| | | | | NPVTSVDAAFR | SEQ ID 174 |
| | | | | VWVYPPEK | SEQ ID 248 |
| KRF-327 | KRPI-327.1 | 5.9 | 47,074 | FNPVTGEVPPR | SEQ ID 73 |
| | | | | NPVTSVDAAFR | SEQ ID 174 |
| | | | | VWVYPPEK | SEQ ID 248 |
| | | | | YYCFQGNK | SEQ ID 267 |
| KRF-327 | KRPI-327.2 | 5.9 | 47,074 | APQVSTPTLVEAAR | SEQ ID 12 |
| | | | | CCSGSLVER | SEQ ID 24 |
| | | | | CCTLPEAQR | SEQ ID 25 |
| | | | | ECCHGDLLECADDR | SEQ ID 46 |
| | | | | SIHTLFGDK | SEQ ID 195 |
| | | | | YLHEVAR | SEQ ID 259 |
| KRF-339 | KRPI-339 | 6.4 | 44,084 | HGGPFCAGDATR | SEQ ID 103 |

As will be evident to one of skill in the art, based upon the present description, a given KRPI can be described according to the data provided for that KRPI in Table VII, VIII, 1× or X. The KRPI is a protein comprising a peptide sequence described for that KRPI (preferably comprising a plurality o$ more preferably all of, the peptide sequences described for that KRPI) and has a pI of about the value stated for that KRPI (preferably within 10%, more preferably within 5% still more preferably within 1% of the stated value) and has a MW of about the value stated for that KRPI (preferably within 10%, more preferably within 5%, still more preferably within 1% of the stated value).

In one embodiment, kidney tissue from a subject is analyzed for quantitative detection of one or more of the following KRPIs: KRPI-2, KRPI-8, KRPI-11, KRPI-13, KRPI-14, KRPI-15, KRPI-16, KRPI-19, KRPI-21, KRPI-23, KRPI-27, KRPI-28, KRPI-35, KRPI-40, KRPI-41, KRPI-42, KRPI-43, KRPI-45.1, KRPI-45.2, KRPI-57, KRPI-59, KRPI-60, KRPI-63, KRPI-70, KRPI-72, KRPI-73, KRPI-76, KRPI-84, KRPI-85, KRPI-86, KRPI-88, KRPI-90, KRPI-91, KRPI-98, KRPI-101, KRPI-104, KRPI-105, KRPI-113, KRPI-122, KRPI-123, KRPI-128, KRPI-131, KRPI-132, KRPI-134, KRPI-138, KRPI-139, KRPI-142, KRPI-143, KRPI-144, KRPI-149, KRPI-152, KRPI-153, KRPI-158, KRPI-159, KRPI-168, KRPI-170, KRPI-178, KRPI-179, KRPI-285, KRPI-286 or any combination of them, wherein a decreased abundance of the KRPI or KRPIs (or any combination of them) in the kidney tissue from the subject relative to kidney tissue from a subject or subjects free from kidney response (e.g., a control sample or a previously determined reference range) indicates the presence of kidney response.

In another embodiment of the invention, kidney tissue from a subject is analyzed for quantitative detection of one or more of the following KRPIs: KRPI-8, KRPI-27, KRPI-28, KRPI-142, KRPI-144, KRPI-149, KRPI-158, KRPI-183, KRPI-184, KRPI-185, KRPI-186, KRPI-188, KRPI-189.1, KRPI-189.2, KRPI-192, KRPI-196, KRPI-202, KRPI-206, KRPI-208, KRPI-210, KRPI-219, KRPI-222, KRPI-229, KRPI-232, KRPI-235.1, KRPI-235.2, KRPI-236, KRPI-237, KRPI-240, KRPI-245, KRPI-247, KRPI-249, KRPI-250, KRPI-252, KRPI-253, KRPI-256, KRPI-257, KRPI-263, KRPI-267, KRPI-273, KRPI-278, KRPI-280, KRPI-282, or any combination of them, wherein an increased abundance of the KRPI or KRPIs (or any combination of them) in kidney tissue from the subject relative to kidney tissue from a subject or subjects free from kidney response (e.g., a control sample or a previously determined reference range) indicates the presence of kidney response.

In a further embodiment, kidney tissue from a subject is analyzed for quantitative detection of (a) one or more KRPIs, or any combination of them, whose decreased abundance indicates the presence of kidney response, i.e., KRPI-2, KRPI-8, KRPI-11, KRPI-13, KRPI-14, KRPI-15, KRPI-16, KRPI-19, KRPI-21, KRPI-23, KRPI-27, KRPI-28, KRPI-35, KRPI-40, KRPI-41, KRPI-42, KRPI-43, KRPI-45.1, KRPI-45.2, KRPI-57, KRPI-59, KRPI-60, KRPI-63, KRPI-70, KRPI-72, KRPI-73, KRPI-76, KRPI-84, KRPI-85, KRPI-86, KRPI-88, KRPI-90, KRPI-91, KRPI-98, KRPI-101, KRPI-104, KRPI-105, KRPI-113, KRPI-122, KRPI-123, KRPI-128, KRPI-131, KRPI-132, KRPI-134, KRPI-138, KRPI-139, KRPI-142, KRPI-143, KRPI-144, KRPI-149, KRPI-152, KRPI-153, KRPI-158, KRPI-159, KRPI-168, KRPI-170, KRPI-178, KRPI-179, KRPI-285, KRPI-286; and (b) one or more KRPIs, or any combination of them, whose increased abundance indicates the presence of kidney response, i.e., KRPI-8, KRPI-27, KRPI-28, KRPI-142, KRPI-144, KRPI-149, KRPI-158, KRPI-183, KRPI-184, KRPI-185, KRPI-186, KRPI-188, KRPI-189.1, KRPI-189.2, KRPI-192, KRPI-196, KRPI-202, KRPI-206, KRPI-208, KRPI-210, KRPI-219, KRPI-222, KRPI-229, KRPI-232, KRPI-235.1, KRPI-235.2, KRPI-236, KRPI-237, KRPI-240, KRPI-245, KRPI-247, KRPI-249, KRPI-250, KRPI-252, KRPI-253, KRPI-256, KRPI-257, KRPI-263, KRPI-267, KRPI-273, KRPI-278, KRPI-280, KRPI-282.

In yet a further embodiment, kidney tissue from a subject is analyzed for quantitative detection of one or more KRPIs and one or more previously known biomarkers of kidney response (e.g., histology, soft tissue imaging). In accordance with this embodiment, the abundance of each KRPI and known biomarker relative to a control or reference range indicates whether a subject has kidney response.

In one embodiment, blood from a subject is analyzed for quantitative detection of KRPI-313, wherein a decreased abundance of the KRPI in the blood from the subject relative to blood from a subject or subjects free from kidney response (e.g., a control sample or a previously determined reference range) indicates the presence of kidney response.

In another embodiment of the invention, blood from a subject is analyzed for quantitative detection of one or more of the following KRPIs: KRPI-314.1, KRPI-314.2, KRPI-327.1, KRPI-327.2, KRPI-339, or any combination of them, wherein an increased abundance of the KRPI or KRPIs (or any combination of them) in blood from the subject relative to blood from a subject or subjects free from kidney response (e.g., a control sample or a previously determined reference range) indicates the presence of kidney response.

In a further embodiment, blood from a subject is analyzed for quantitative detection of (a) one or more KRPIs, or any combination of them, whose decreased abundance indicates the presence of kidney response, i.e., KRPI-313; and (b) one or more KRPIs, or any combination of them, whose increased abundance indicates the presence of kidney response, i.e., KRPI-314.1, KRPI-314.2, KRPI-327.1, KRPI-327.2, KRPI-339.

In yet a further embodiment, blood from a subject is analyzed for quantitative detection of one, or more KRPIs and one or more previously known biomarkers of kidney response (e.g., histology, soft tissue imaging). In accordance with this embodiment, the abundance of each KRPI and known biomarker relative to a control or reference range indicates whether a subject has kidney response.

Preferably, the abundance of a KRPI is normalized to an Expression Reference Protein Isoform (ERPI). ERPIs (examples listed in Table XI) can be identified by partial amino acid sequencing of ERFs, which are described above (Tables V and VI), using the methods and apparatus of the Preferred Technology.

TABLE XI

Expression Reference Protein Isoforms (ERPIs) in Kidney Tissue

| Table XI | | | | Amino Acid Sequences of | |
|---|---|---|---|---|---|
| ERF | ERPI | pI | MW (Da) | Tryptic Digest Peptides | Seq ID |
| ERF-1 | ERPI-1 | 5.32 | 20135 | VDYGGVTVDELGK | SEQ ID 233 |
| | | | | VLTPTQVMNR | SEQ ID 240 |
| | | | | GNDISSGTVLSEYVGSGPPK | SEQ ID 95 |
| | | | | LYTLVLTDPDAPSR | SEQ ID 154 |
| ERF-2 | ERPI-2.1 | 4.61 | 28930 | YLAEFATGNDR | SEQ ID 268 |
| | | | | HLIPAANTGESK | SEQ ID 269 |
| | | | | EAAENSLVAYK | SEQ ID 270 |
| | | | | VAGMDVELTVEER | SEQ ID 271 |
| | | | | NLLSVAYK | SEQ ID 169 |
| ERF-2 | ERPI-2.2 | 4.61 | 28930 | CNFYDNK | SEQ ID 272 |

As shown above, the KRPs described herein include previously known proteins, as well as variants of known proteins where the variants were not previously known to be associated with kidney response. For each KRPI, the present invention additionally provides: (a) a preparation comprising the isolated KRPI; (b) a preparation comprising one or more fragments of the KRPI; and (c) antibodies that bind to said KRPI, to said fragments, or both to said KRPI and to said fragments. As used herein, a KRPI is “isolated” when it is present in a preparation that is substantially free of contaminating proteins, i.e., a preparation in which less than 10% (preferably less than 5%, more preferably less than 1%) of the total protein present is contaminating protein(s). A contaminating protein is a protein or protein isoform having a significantly different pI or MW from those of the isolated KRPI, as determined by 2D electrophoresis. As used herein, a “significantly different” pI or MW is one that permits the contaminating protein to be resolved from the KRPI on 2D electrophoresis, performed according to the Reference Protocol.

In one embodiment, an isolated protein is provided, said protein comprising a peptide with the amino acid sequence identified in Table VII, VIII, 1× or X for a KRPI, said protein having a pI and MW within 10% (preferably within 5%, more preferably within 1%) of the values identified in Table VII, VIII, 1× or X for that KRPI.

The KRPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the KRPIs are separated on a 2-D gel by virtue of their MWs and pIs and visualized by staining the gel. In one embodiment, the KRPIs are stained with a fluorescent dye and imaged with a fluorescence scanner. Sypro Red (Molecular Probes, Inc., Eugene, Oreg.) is a suitable dye for this purpose. A preferred fluorescent dye is Pyridinium, 4-[2-[4-(dipentylamino)-2-trifluoromethylphenyl]ethenyl-]-(sulfobutyl)-, inner salt. See U.S. application Ser. No. 09/412,168, filed on Oct. 5, 1999, which is incorporated herein by reference in its entirety.

Alternatively, KRPIs can be detected in an immunoassay. In one embodiment, an immunoassay is performed by contacting a sample from a subject to be tested with an anti-KRPI antibody under conditions such that immunospecific binding can occur if the KRPI is present, and detecting or measuring the amount of any immunospecific binding by the antibody. Preferably, the anti-KRPI antibody preferentially binds to the KRPI rather than to other isoforms of the same protein. Anti-KRPI antibodies can be produced by the methods and techniques described herein; examples of such antibodies known in the art which have been reported to recognize a protein having an amino acid sequence of a KRPI, or which have been reported to recognize a protein named in the database selected by searching with the KRPI sequence corresponding to a sequence of a KRPI, are set forth in Table XII. These antibodies shown in Table XII are already reported to bind to the protein of which the KRPI is itself predicted to be a family member. Particularly, the anti-KRPI antibody preferentially binds to the KRPI rather than to other variants of the same protein.

TABLE XII
Known Antibodies That Recognize KRPI or KRPI-Related Polypeptides

| | | | Human | | | |
|---|---|---|---|---|---|---|
| | | Rat | Homologue | | | |
| | | Accession | accession | | | Catalogue |
| KRF | KRPI | number | number | Antibody | Manufacturer | Number |
| KRF- | KRPI- | P02770 | P02768 | Albumin, Human, | ACCURATE | IMS-01-026- |
| 101 | 101 | | | Chicken anti- | CHEMICAL & | 02 |
| | | | | | SCIENTIFIC | |
| | | | | | CORPORATION | |
| KRF- | KRPI- | P26040 | P15311 | Mouse monoclonal | Lab Vision | MS-661-P0 |
| 142 | 142 | | | anti-ezrin | Corporation | |
| KRF- | KRPI- | P04797 | P04406 | Glyceraldehyde-3- | BIODESIGN | H86504M |
| 210 | 210 | | | Phosphate | INTERNATIONAL | |
| | | | | Dehydrogenase | | |
| KRF- | KRPI- | P14480 | P02675 | Fibrinogen, Fibrin | ACCURATE | NYB-18C6 |
| 222 | 222 | | | I, B-beta chain (Bβ | CHEMICAL & | |
| | | | | 1-42), Clone: | SCIENTIFIC | |
| | | | | 18C6, Mab anti- | CORPORATION | |
| | | | | Human | | |
| KRF- | KRPI- | P20059 | P02790 | Goat anti- | STRATEGIC | |
| 313 | 313 | | | Hemopexin, | BIOSOLUTIONS | |

* Further information about these antibodies can be obtained from their commercial sources at: Abcam Ltd - http://www.abcam.com ACCURATE CHEMICAL & SCIENTIFIC CORPORATION http://www.accuratechemical.com/; BIODESIGN INTERNATIONAL - http://www.biodesign.com/; Lab Vision Corporation - http://www.labvision.com; STRATEGIC BIOSOLUTIONS - http://www.strategicbiosolutions.com.

In a particular embodiment, the anti-KRPI antibody binds to the KRPI with at least 2-fold greater affinity, more preferably at least 5-fold greater affinity, still more preferably at least 10-fold greater affinity, than to said other isoforms of the same protein. When the antibodies in Table XII do not display the required preferential selectivity for the target KRPI, one skilled in the art can generate additional antibodies by using the KRPI itself for the generation of such antibodies.

KRPIs can be transferred from the gel to a suitable membrane (e.g. a PVDF membrane) and subsequently probed in suitable assays that include, without limitation, competitive and non-competitive assay systems using techniques such as western blots and “sandwich” immunoassays using anti-KRPI antibodies which can be identified as described herein, or others raised against the KRPIs of interest. The immunoblots can be used to identify those anti-KRPI antibodies displaying the selectivity required to immuno-specifically differentiate a KRPI from other isoforms encoded by the same gene.

In one embodiment, binding of antibody in tissue sections can be used to detect aberrant KRPI localization or an aberrant level of one or more KRPIs in a specific embodiment, antibody to a KRPI can be used to assay a tissue sample (e.g., a kidney biopsy) from a subject for the level of the KRPI where an aberrant level of KRPI is indicative of kidney response. As used herein, an “aberrant level” means a level that is increased or decreased compared with the level in a subject free from kidney response or a reference level. If desired, the comparison can be performed with a matched sample from the same subject, taken from a portion of the body not affected by kidney response.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a KRPI can be detected in a fluid sample (e.g., spinal fluid, blood, plasma, urine, or tissue homogenate) by means of a two-step sandwich assay. In the first step, a capture reagent (e.g., an anti-KRPI antibody) is used to capture the KRPI. Examples of such antibodies known in the art can be identified as described infra. The capture reagent can optionally be immobilized on a solid phase. In the second step, a directly or indirectly labeled detection reagent is used to detect the captured KRPI. In one embodiment, the detection reagent is a lectin. Any lectin can be used for this purpose that preferentially binds to the KRPI rather than to other isoforms that have the same core protein as the KRPI or to other proteins that share the antigenic determinant recognized by the antibody. In a preferred embodiment, the chosen lectin binds to the KRPI with at least 2-fold greater affinity, more preferably at least 5-fold greater affinity, still more preferably at least 10-fold greater affinity, than to said other isoforms that have the same core protein as the KRPI or to said other proteins that share the antigenic determinant recognized by the antibody. Based on the present description, a lectin that is suitable for detecting a given KRPI can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the KRPI in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding a KRPI, a related gene, or related nucleic acid sequences or subsequences, including complementary sequences, can also be used in hybridization assays. A nucleotide encoding a KRPI, or subsequences thereof comprising at least 8 nucleotides, preferably at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of genes encoding KRPIs, or for differential diagnosis of subjects with signs or symptoms suggestive of kidney response. In particular, such a hybridization assay can be carried out by a method comprising contacting a subject's sample containing nucleic acid with a nucleic acid probe capable of hybridizing to a DNA or RNA that encodes a KRPI, under conditions such that hybridization can occur, and detecting or measuring any resulting hybridization. Nucleotides can be used for therapy of subjects having kidney response, as described below.

The invention also provides diagnostic kits, comprising an anti-KRPI antibody. In addition, such a kit may optionally comprise one or more of the following: (1) instructions for using the anti-KRPI antibody for diagnosis, prognosis, therapeutic monitoring or any combination of these applications; (2) a labeled binding partner to the antibody; (3) a solid phase (such as a reagent strip) upon which the anti-KRPI antibody is immobilized; and (4) a label or insert indicating regulatory approval for diagnostic, prognostic or therapeutic use or any combination thereof. If no labeled binding partner to the antibody is provided, the anti-KRPI antibody itself can be labeled with a detectable marker, e.g., a chemiluminescent, enzymatic, fluorescent, or radioactive moiety.

The invention also provides a kit comprising a nucleic acid probe capable of hybridizing to RNA encoding a KRPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a KRPI, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

Kits are also provided which allow for the detection of a plurality of KRPIs or a plurality of nucleic acids each encoding a KRPI. A kit can optionally further comprise a predetermined amount of an isolated KRPI protein or a nucleic acid encoding a KRPI, e.g., for use as a standard or control.

Statistical Techniques for Identifying KRPIs and KRPI Clusters

The uni-variate differential analysis tools, such as fold changes, wilcoxon rank sum test and t-test, are useful in identifying individual KRFs or KRPIs that are diagnostically associated with kidney response or in identifying individual KRPIs that regulate the disease process. In most cases, however, those skilled in the art appreciate that the disease process is associated with a combination of KRFs or KRPIs (and to be regulated by a combination of KRPIs), rather than individual KRFs and KRPIs in isolation. The strategies for discovering such combinations of KRFs and KRPIs differ from those for discovering individual KRFs and KRPIs. In such cases, each individual KRF and KRPI can be regarded as one variable and the disease can be regarded as a joint, multi-variate effect caused by interaction of these variables.

The following steps can be used to identify markers from data produced by the Preferred Technology.

The first step is to identify a collection of KRFs or KRPIs that individually show significant association with kidney response. The association between the identified KRFs or KRPIs and kidney response need not be as highly significant as is desirable when an individual KRF or KRPI is used as a diagnostic. Any of the tests discussed above (fold changes, wilcoxon rank sum test, etc.) can be used at this stage. Once a suitable collection of KRFs or KRPIs has been identified, a sophisticated multi-variate analysis capable of identifying clusters can then be used to estimate the significant multivariate associations with kidney response.

Linear Discriminant Analysis (LDA) is one such procedure, which can be used to detect significant association between a cluster of variables (i.e., KRFs or KRPIs) and kidney response. In performing LDA, a set of weights is associated with each variable (i.e., KRF or KRPI) so that the linear combination of weights and the measured values of the variables can identify the disease state by discriminating between subjects having kidney response and subjects free from kidney response. Enhancements to the LDA allow stepwise inclusion (or removal) of variables to optimize the discriminant power of the model. The result of the LDA is therefore a cluster of KRFs or KRPIs which can be used, without limitation, for diagnosis, prognosis, therapy or drug development. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of KRFs or KRPIs can be identified by qualitative measures by comparing the percentage feature presence of a KRF or KRPI of one group of samples (e.g., samples from diseased subjects) with the percentage feature presence of a KRF or KRPI in another group of samples (e.g., samples from control subjects). The “percentage feature presence” of a KRF or KRPI is the percentage of samples in a group of samples in which the KRF or KRPI is detectable by the detection method of choice. For example, if a KRF is detectable in 95 percent of samples from diseased subjects, the percentage feature presence of that KRF in that sample group is 95 percent. If only 5 percent of samples from non-diseased subjects have detectable levels of the same KRF, detection of that KRF in the sample of a subject would suggest that it is likely that the subject suffers from kidney response.

Use in Clinical Studies

The diagnostic methods and compositions of the present invention can assist in monitoring a clinical study, e.g. to evaluate drugs for therapy of kidney response. In one embodiment, candidate molecules are tested for their ability to restore KRF or KRPI levels in a subject having kidney response to levels found in subjects free from kidney response or, in a treated subject (e.g. after treatment with a toxic agent), to preserve KRF or KRPI levels at or near non-kidney response values. The levels of one or more KRFs or KRPIs can be assayed.

In another embodiment, the methods and compositions of the present invention are used to screen candidates for a clinical study to identify individuals having kidney response; such individuals can then be either excluded from or included in the study or can be placed in a separate cohort for treatment or analysis. If desired, the candidates can concurrently be screened to identify individuals with elevated alanine aminotransferase and/or aspartate aminotransferase levels; procedures for these screens are well known in the art.

Purification of KRPIs

In particular aspects, the invention provides isolated mammalian KRPIs, preferably human KRPIs, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) KRPI, e.g., binding to a KRPI substrate or KRPI binding partner, antigenicity (binding to an anti-KRPI antibody), immunogenicity, enzymatic activity and the like.

In specific embodiments, the invention provides fragments of a KRPI comprising at least 5 amino acids, at least 10 amino acids, at least 50 amino acids, or at least 75 amino acids. Fragments lacking some or all of the regions of a KRPI are also provided, as are proteins (e.g., fusion proteins) comprising such fragments. Nucleic acids encoding the foregoing are provided.

Once a recombinant nucleic acid which encodes the KRPI, a portion of the KRPI, or a precursor of the KRPI is identified, the gene product can be analyzed. This is achieved by assays based on the physical or functional properties of the product, including radioactive labeling of the product followed by analysis by gel electrophoresis, immunoassay, etc.

The KRPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the KRPI is identified, the entire amino acid sequence of the KRPI can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native KRPIs can be purified from natural sources, by standard methods such as those described above (e.g., immunoaffinity purification).

In a preferred embodiment, KRPIs are isolated by the Preferred Technology described supra. For preparative-scale runs, a narrow-range “zoot gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated KRPI that can be recovered from the gel. When used in this way for preparative-scale runs, the Preferred Technology typically provides up to 100 ng, and can provide up to 1000 ng, of an isolated KRPI in a single run. Those of skill in the art will appreciate that a zoom gel can be used in any separation strategy which employs gel isoelectric focusing.

The invention thus provides an isolated KRPI, an isolated KRPI-related polypeptide, and an isolated derivative or fragment of a KRPI or a KRPI-related polypeptide; any of the foregoing can be produced by recombinant DNA techniques or by chemical synthetic methods.

Isolation of DNA Encoding a KRPI

Specific embodiments for the cloning of a gene encoding a KRPI, are presented below by way of example and not of limitation.

The nucleotide sequences of the present invention, including DNA and RNA, and comprising a sequence encoding a KRPI or a fragment thereof, or a KRPI-related polypeptide, may be synthesized using methods known in the art, such as using conventional chemical approaches or polymerase chain reaction (PCR) amplification. The nucleotide sequences of the present invention also permit the identification and cloning of the gene encoding a KRPI homolog or KRPI ortholog including, for example, by screening cDNA libraries, genomic libraries or expression libraries.

For example, to clone a gene encoding a KRPI by PCR techniques, anchored degenerate oligonucleotides (or a set of most likely oligonucleotides) can be designed for all KRPI peptide fragments identified as part of the same protein. PCR reactions under a variety of conditions can be performed with relevant cDNA and genomic DNAs (e.g., from kidney tissue or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer K D, Biotechniques, 1995, 19(4):550-2). Vectorette PCR may be performed with probes that are, for example, anchored degenerate oligonucleotides (or most likely oligonucleotides) coding for KRPI peptide fragments, using as a template a genomic library or cDNA library pools.

Anchored degenerate oligonucleotides (and most likely oligonucleotides) can be designed for all KRPI peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding a KRPI or KRPI fragment of the present invention are useful for their ability to hybridize selectively with complementary stretches of genes encoding other proteins. Depending on the application, a variety of hybridization conditions may be employed to obtain nucleotide sequences at least 30%, 35%, 40%, 45%, 50%, 55%, 60%, 65%, 70%, 75%, 80%, 85%, 90%, 95% or 99% identical, or 100% identical, to the sequence of a nucleotide encoding a KRPI.

For a high degree of selectivity, relatively stringent conditions are used to form the duplexes, such as low salt or high temperature conditions. As used herein, “highly stringent conditions” means hybridization to filter-bound DNA in 0.5 M NaHPO₄, 7% sodium dodecyl sulfate (SDS), 1 mM EDTA at 65_C, and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding a KRPI, 37° C. for 90 to 95% identity and 32° C. for 70 to 90% identity.

In the preparation of genomic libraries, DNA fragments are generated, some of which will encode parts or the whole of a KRPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T₄, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened with labeled degenerate oligonucleotide probes corresponding to the amino acid sequence of any peptide of the KRPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

In Tables VII, VIII, 1× and X above, some KRPIs disclosed herein were found to correspond to isoforms of previously identified proteins encoded by genes whose sequences are publicly known. (Sequence analysis and protein identification of KRPIs was carried out using the methods described in Section 6.1.14). To screen such a gene, any probe may be used that is complementary to the gene or its complement; preferably the probe is 10 nucleotides or longer, more preferably 15 nucleotides or longer.

The SWISS-PROT and trEMBL databases (held by the Swiss Institute of Bioinformatics (SIB) and the European Bioinformatics Institute (EBI) which are available at http://www.expasy.ch/) and the GenBank database (held by the National Institute of Health (N1H) which is available at http://www.ncbi.nlm.nih.gov/) provide protein sequences for the KRPIs listed in Tables VII, VIII, 1× or X under the following accession numbers and each sequence is incorporated herein by reference (see Table XIII). In many cases the protein sequence in the database will cross reference a nucleic acid or gene sequence encoding the protein or related protein

TABLE XIII
Nucleotide sequences encoding KRPIs,
KRPI Related Proteins or ERPIs

| | | | Human |
|---|---|---|---|
| | | Rat/mouse | Homologue |
| Feature | Isoform | Accession | Accession |
| KRF | KRPI | Number | Number |
| KRF-2 | KRPI-2 | P51635 | P14550 |
| KRF-8 | KRPI-8 | BAB21527* | P52565 |
| KRF-11 | KRPI-11 | P19112 | P09467 |
| KRF-13 | KRPI-13 | P02770 | P02768 |
| KRF-14 | KRPI-14 | P24329 | Q16762 |
| KRF-15 | KRPI-15 | P48508 | P48507 |
| KRF-16 | KRPI-16 | P07724* | P02768 |
| KRF-19 | KRPI-19 | P14942 | — |
| KRF-21 | KRPI-21 | 6636119 | O43707 |
| KRF-23 | KRPI-23 | P15999 | P25705 |
| KRF-27 | KRPI-27 | P14668 | P08758 |
| KRF-28 | KRPI-28 | 3766203* | AAH07716.1 |
| KRF-35 | KRPI-35 | 3435296 | P32754 |
| KRF-40 | KRPI-40 | 6636119 | O43707 |
| KRF-41 | KRPI-41 | P02770 | P02768 |
| KRF-42 | KRPI-42 | AAH03981 | P06733 |
| KRF-43 | KRPI-43 | 3747085 | P40925 |
| KRF-45 | KRPI-45.1 | P11348 | P09417 |
| KRF-45 | KRPI-45.2 | P10860 | P49448 |
| KRF-57 | KRPI-57 | Q07523 | Q9NYQ3 |
| KRF-59 | KRPI-59 | P70473 | Q9UHK6 |
| KRF-60 | KRPI-60 | P31044 | P30086 |
| KRF-63 | KRPI-63 | — | P13639 |
| KRF-70 | KRPI-70 | P32551 | P22695 |
| KRF-72 | KRPI-72 | P51635 | P14550 |
| KRF-73 | KRPI-73 | Q02253 | Q02252 |
| KRF-76 | KRPI-76 | 118542* | P49448 |
| KRF-84 | KRPI-84 | P15999 | P25705 |
| KRF-85 | KRPI-85 | Q02253 | Q02252 |
| KRF-86 | KRPI-86 | Q07523 | Q9NYQ3 |
| KRF-88 | KRPI-88 | P51635 | P14550 |
| KRF-90 | KRPI-90 | P00507 | P00505 |
| KRF-91 | KRPI-91 | P07632 | P00441 |
| KRF-98 | KRPI-98 | P31399 | O75947 |
| KRF-101 | KRPI-101 | P02770 | P02768 |
| KRF-104 | KRPI-104 | P10860 | P49448 |
| KRF-105 | KRPI-105 | Q64718 | Q05639 |
| KRF-113 | KRPI-113 | O35078 | — |
| KRF-122 | KRPI-122 | P12075 | P10606 |
| KRF-123 | KRPI-123 | 3462887 | — |
| KRF-128 | KRPI-128 | Q9WVK7 | — |
| KRF-131 | KRPI-131 | P08109* | P11142 |
| KRF-132 | KRPI-132 | P10860 | P49448 |
| KRF-134 | KRPI-134 | P07150 | P04083 |
| KRF-138 | KRPI-138 | 13435897* | Q15365 |
| KRF-139 | KRPI-139 | P02535* | P13645 |
| KRF-139 | KRPI-285 | AF284573* | 9367116 |
| KRF-139 | KRPI-286 | Q07523 | Q9UJM8 |
| KRF-140 | KRPI-140 | 12229956 | AAF72806.1 |
| KRF-142 | KRPI-142 | P26040* | P15311 |
| KRF-143 | KRPI-143 | P12346 | P02787 |
| KRF-144 | KRPI-144 | 203341 | P09668 |
| KRF-149 | KRPI-149 | P10860 | P49448 |
| KRF-152 | KRPI-152 | 71620* | P02571 |
| KRF-153 | KRPI-153 | Q11136* | P12955 |
| KRF-158 | KRPI-158 | P02770 | P02768 |
| KRF-159 | KRPI-159 | P15651 | P16219 |
| KRF-168 | KRPI-168 | 3747085 | P40925 |
| KRF-170 | KRPI-170 | Q60932* | P21796 |
| KRF-178 | KRPI-178 | P48500 | P00938 |
| KRF-179 | KRPI-179 | P15999 | P25705 |
| KRF-183 | KRPI-183 | P05065 | P04075 |
| KRF-184 | KRPI-184 | P04182 | P04181 |
| KRF-185 | KRPI-185 | 3766203* | — |
| KRF-186 | KRPI-186 | P14152* | P40925 |
| KRF-188 | KRPI-188 | AAH05631* | Q9BQ75 |
| KRF-189 | KRPI-189.1 | P51635 | P14550 |
| KRF-189 | KRPI-189.2 | O35078 | P14920 |
| KRF-192 | KRPI-192 | Q60932* | P21796 |
| KRF-196 | KRPI-196 | 1051270 | — |
| KRF-202 | KRPI-202 | P46953 | P46952 |
| KRF-203 | KRPI-203 | P13803 | P13804 |
| KRF-206 | KRPI-206 | Q02253 | Q02252 |
| KRF-208 | KRPI-208 | P04166 | O43169 |
| KRF-210 | KRPI-210 | P04797 | P04406 |
| KRF-219 | KRPI-219 | P19804 | O60361 |
| KRF-222 | KRPI-222 | P14480 | P02675 |
| KRF-225 | KRPI-225 | P20673 | P04424 |
| KRF-229 | KRPI-229 | P20788 | P47985 |
| KRF-232 | KRPI-232 | P25093 | P16930 |
| KRF-234 | KRPI-234 | Q60648* | P17900 |
| KRF-235 | KRPI-235.1 | P41562 | O475874 |
| KRF-235 | KRPI-235.2 | O55171 | — |
| KRF-236 | KRPI-236 | P02761 | — |
| KRF-237 | KRPI-237 | 2143765 | P09210 |
| KRF-240 | KRPI-240 | P17080 | P17080 |
| KRF-245 | KRPI-245 | P38983 | P08865 |
| KRF-247 | KRPI-247 | P04762 | P04040 |
| KRF-249 | KRPI-249 | P08109* | P11142 |
| KRF-250 | KRPI-250 | P04762 | P04040 |
| KRF-252 | KRPI-252 | 13542680* | P05217 |
| KRF-253 | KRPI-253 | Q64718 | Q05639 |
| KRF-256 | KRPI-256 | 8307686 | — |
| KRF-257 | KRPI-257 | 1196815 | — |
| KRF-263 | KRPI-263 | Q64644 | Q9NY17 |
| KRF-266 | KRPI-266 | P80314* | P78371 |
| KRF-267 | KRPI-267 | AAH03328 | O95865 |
| KRF-273 | KRPI-273 | P21107* | P12324 |
| KRF-278 | KRPI-278 | P12346 | P02787 |
| KRF-280 | KRPI-280 | P32755 | P32754 |
| KRF-282 | KRPI-282 | P13832 | P19105 |
| KRF-313 | KRPI-313 | P20059 | P02790 |
| KRF-314 | KRPI-314.1 | P02770 | P02768 |
| KRF-314 | KRPI-314.2 | P20059 | P02790 |
| KRF-327 | KRPI-327.1 | P20059 | P02790 |
| KRF-327 | KRPI-327.2 | P02770 | P02768 |
| KRF-339 | KRPI-339 | P11680* | P27918 |

For any KRPI, degenerate probes, or probes taken from the sequences described above by accession number may be used for screening. In the case of degenerate probes, they can be constructed from the partial amino sequence information obtained from tandem mass spectra of tryptic digest peptides of the KRPI. To screen such a gene, any probe may be used that is complementary to the gene or its complement; preferably the probe is 10 nucleotides or longer, more preferably 15 nucleotides or longer. When a library is screened, clones with insert DNA encoding the KRPI or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the washing conditions described supra for highly stringent or moderately stringent hybridization.

In yet another aspect of the invention, clones containing nucleotide sequences encoding the entire KRPI, a fragment of a KRPI, a KRPI-related polypeptide, or a fragment of a KRPI-related polypeptide any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed KRPI or KRPI-related polypeptides. In one embodiment, the various anti-KRPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes a KRPI, a fragment of a KRPI, a KRPI-related polypeptide, or a fragment of a KRPI-related polypeptide can be detected using DYNA Beads according to Olsvick et al., 29th ICAAC, Houston, Tex. 1989, incorporated herein by reference. Anti-KRPI antibodies are crosslinked to tosylated DYNA Beads M280, and these antibody-containing beads are then contacted with colonies or plaques expressing recombinant polypeptides. Colonies or plaques expressing a KRPI or KRPI-related polypeptide are identified as any of those that bind the beads.

Alternatively, the anti-KRPI antibodies can be nonspecifically immobilized to a suitable support, such as silica or Celite⁷ resin. This material is then used to adsorb to bacterial colonies expressing the KRPI protein or KRPI-related polypeptide as described herein.

In another aspect, PCR amplification may be used to isolate from genomic DNA a substantially pure DNA (i.e., a DNA substantially free of contaminating nucleic acids) encoding the entire KRPI or a part thereof. Preferably such a DNA is at least 95% pure, more preferably at least 99% pure. Oligonucleotide sequences, degenerate or otherwise, that correspond to peptide sequences of KRPIs disclosed herein can be used as primers.

PCR can be carried out, e.g., by use of a Perkin-Elmer Cetus thermal cycler and Taq polymerase (Gene Amp⁷ or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding a KRPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding a KRPI can also be identified by mRNA selection by nucleic acid hybridization followed by in vitro translation. In this procedure, fragments are used to isolate complementary mRNAs by hybridization. Such DNA fragments may represent available, purified DNA encoding a KRPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize a KRPI. A radiolabelled cDNA encoding a KRPI can be synthesized using the selected mRNA (from the adsorbed polysomes) as a template. The radiolabelled mRNA or cDNA may then be used as a probe to identify the DNA fragments encoding a KRPI from among other genomic DNA fragments.

Alternatives to isolating genomic DNA encoding a KRPI include, but are not limited to, chemically synthesizing the gene sequence itself from a known sequence or making cDNA to the mRNA which encodes the KRPI. For example, RNA for cDNA cloning of the gene encoding a KRPI can be isolated from cells which express the KRPI. Those skilled in the art will understand from the present description that other methods may be used and are within the scope of the invention.

Any suitable eukaryotic cell can serve as the nucleic acid source for the molecular cloning of the gene encoding a KRPI. The nucleic acid sequences encoding the KRPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, IT.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the only limitation is that the vector system chosen be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding a KRPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the KRPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native KRPIs, nucleotide sequences encoding amino acid sequences with functionally equivalent amino acids, nucleotide sequences encoding KRPIs, a fragments of KRPIs, KRPI-related polypeptides, or fragments of KRPI-related polypeptides.

In a specific embodiment, an isolated nucleic acid molecule encoding a KRPI-related polypeptide can be created by introducing one or more nucleotide substitutions, additions or deletions into the nucleotide sequence of a KRPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, bistidine), acidic side chains (e.g., aspartic acid, glutamic acid), uncharged polar side chains (e.g., glycine, asparagine, glutamine, serilie, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Expression of DNA Encoding KRPIs

The nucleotide sequence coding for a KRPI, a KRPI analog, a KRPI-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the KRPI or its flanking regions, or the native gene encoding the KRPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human KRPI) is expressed. In yet another embodiment, a fragment of a KRPI comprising a domain of the KRPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding a KRPI or fragment thereof may be regulated by a second nucleic acid sequence so that the KRPI or fragment is expressed in a host transformed with the recombinant DNA molecule. For example, expression of a KRPI may be controlled by any promoter or enhancer element known in the art. Promoters which may be used to control the expression of the gene encoding a KRPI or a KRPI-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the _-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus ³⁵S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al, 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to a KRPI-encoding nucleic acid, one or more origins of replication, and, optionally, one or more selectable markers (e.g., an antibiotic resistance gene).

In a specific embodiment, an expression construct is made by subcloning a KRPI or a KRPI-related polypeptide coding sequence into the EcoRI restriction site of each of the three pGEX vectors (Glutathione S-Transferase expression vectors; Smith and Johnson, 1988, Gene 7:31-40). This allows for the expression of the KRPI product or KRPI-related polypeptide from the subclone in the correct reading frame.

In mammalian host cells, a number of viral-based expression systems may be utilized. In cases where an adenovirus is used as an expression vector, the KRPI coding sequence or KRPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzytnol. 153:51-544).

Expression vectors containing inserts of a gene encoding a KRPI or a KRPI-related polypeptide can be identified by three general approaches: (a) nucleic acid hybridization, (b) presence or absence of “marker” gene functions, and (c) expression of inserted sequences. In the first approach, the presence of a gene encoding a KRPI inserted in an expression vector can be detected by nucleic acid hybridization using probes comprising sequences that are homologous to an inserted gene encoding a KRPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a KRPI in the vector. For example, if the gene encoding the KRPI is inserted within the marker gene sequence of the vector, recombinants containing the gene encoding the KRPI insert can be identified by the absence of the marker gene function. In the third approach, recombinant expression vectors can be identified by assaying the gene product (i.e., KRPI) expressed by the recombinant. Such assays can be based, for example, on the physical or functional properties of the KRPI in in vitro assay systems, e.g., binding with anti-SPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered KRPI or KRPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, HEK293, 3T3, W138, and in particular, endothelial cell lines, and normal human cell lines such as, for example, normal human endothelial cells. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other specific embodiments, the KRPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding a KRPI, a fragment of a KRPI, a KRPI-related polypeptide, or a fragment of a KRPI-related polypeptide can fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

Fusion proteins can be made by ligating the appropriate nucleic acid sequences encoding the desired amino acid sequences to each other by methods known in the art, in the proper coding frame, and expressing the chimeric product by methods commonly known in the art. Alternatively, a fusion protein may be made by protein synthetic techniques, e.g., by use of a peptide synthesizer.

Both cDNA and genomic sequences can be cloned and expressed.

Domain Structure of KRPIs

Domains of some KRPIs are known in the art and have been described in the scientific literature. Moreover, domains of a KRPI can be identified using techniques known to those of skill in the art. For example, one or more domains of a KRPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html. Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occurring transmembrane proteins (see, e.g., http://www.ch.embnet. org/software/TMPRED form.html; Hofmann & Stoffel. (1993) ATMbase—A database of membrane spanning proteins segments Biol. Chem. Hoppe-Seyler 347,166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, the skilled artisan can identify domains of a KRPI having enzymatic or binding activity, and further can identify nucleotide sequences encoding such domains. These nucleotide sequences can then be used for recombinant expression of a KRPI fragment that retains the enzymatic or binding activity of the KRPI.

Based on the present description, the skilled artisan can identify domains of a KRPI having enzymatic or binding activity, and further can identify nucleotide sequences encoding such domains. These nucleotide sequences can then be used for recombinant expression of KRPI fragments that retain the enzymatic or binding activity of the KRPI.

In one embodiment, a KRPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

A KRPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay. In a preferred embodiment, the function of a domain of a KRPI is determined using an assay.

Production of Antibodies to KRPIs

According to the invention a KRPI, KRPI analog, KRPI-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab=) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognize gene products of genes encoding KRPIs may be prepared. Certain antibodies are already known and can be purchased from commercial sources as shown in Table XII.

In another embodiment, methods known to those skilled in the art are used to produce antibodies that recognize a KRPI, a KRPI analog, a KRPI-related polypeptide, or a derivative or fragment of any of the foregoing.

In one embodiment of the invention, antibodies to a specific domain of a KRPI are produced. In a specific embodiment, hydrophilic fragments of a KRPI are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of a KRPI, one may assay generated hybridomas for a product which binds to a KRPI fragment containing such domain. For selection of an antibody that specifically binds a first KRPI homolog but which does not specifically bind to (or binds less avidly to) a second KRPI homolog, one can select on the basis of positive binding to the first KRPI homolog and a lack of binding to (or reduced binding to) the second KRPI homolog. Similarly, for selection of an antibody that specifically binds a KRPI but which does not specifically bind to (or binds less avidly to) a different isoform of the same protein (such as a different glycoform having the same core peptide as the KRPI), one can select on the basis of positive binding to the KRPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to a KRPI than to a different isoform or isoforms (e.g., glycoforms) of the KRPI.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a KRPI, a fragment of a KRPI, a KRPI-related polypeptide, or a fragment of a KRPI-related polypeptide. In a particular embodiment, rabbit polyclonal antibodies to an epitope of a KRPI or a KRPI-related polypeptide can be obtained. For example, for the production of polyclonal or monoclonal antibodies, various host animals can be immunized by injection with the native or a synthetic (e.g., recombinant) version of a KRPI, a fragment of a KRPI, a KRPI-related polypeptide, or a fragment of a KRPI-related polypeptide, including but not limited to rabbits, mice, rats, etc. The Preferred Technology described herein provides isolated KRPIs suitable for such immunization. If the KRPI is purified by gel electrophoresis, the KRPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward a KRPI, a fragment of a KRPI, a KRPI-related polypeptide, or a fragment of a KRPI-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a KRPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labelled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 1879-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this, asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology, 1986, 121:210.

The invention provides functionally active fragments, derivatives or analogs of the anti-KRPI immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e., by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the KRPIs of the invention, e.g., for imaging these proteins, measuring levels thereof in appropriate physiological samples, in diagnostic methods, etc.

Expression of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 198, Gene 45:101; Cockett et al., 1990, Bio/Technology 8:2).

A variety of host-expression vector systems may be utilized to express an antibody molecule of the invention. Such host-expression systems represent vehicles by which the coding sequences of interest maybe produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (G ST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccilia virus are loaded onto Ni2+ nitriloacetic acid-agarose columns and histidine-tagged proteins are selectively eluted with imidazole-containing buffers.

Conjugated Antibodies

In a preferred embodiment, anti-KRPI antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinyl amine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

An anti-KRPI antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-blood), granulocyte colony stimulating factor (G-blood), nerve growth factor (NGF) or other growth factor. Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

Diagnosis of Kidney Response

In accordance with the present invention, test samples of blood, serum, plasma, urine or kidney tissue are obtained from a subject suspected of having or known to have kidney response can be used for diagnosis or monitoring. In one embodiment, a decreased abundance of one or more KRFs or KRPIs (or any combination of them) in a test sample relative to a control sample (from a subject or subjects free from kidney response) or a previously determined reference range indicates the presence of kidney response; KRFs and KRPIs suitable for this purpose are identified in Tables I, III, VII and IX, respectively, as described in detail above. In another embodiment of the invention, an increased abundance of one or more KRFs or KRPIs (or any combination of them) in a test sample compared to a control sample or a previously determined reference range indicates the presence of kidney response; KRFs and KRPIs suitable for this purpose are identified in Tables II, IV, VIII and X respectively, as described in detail above. In another embodiment, the relative abundance of one or more KRFs or KRPIs (or any combination of them) in a test sample compared to a control sample or a previously determined reference range indicates a subtype of kidney response (e.g., familial or sporadic kidney response). In yet another embodiment, the relative abundance of one or more KRFs or KRPIs (or any combination of them) in a test sample relative to a control sample or a previously determined reference range indicates the degree or severity of kidney response. In any of the aforesaid methods, detection of one or more KRPIs described herein may optionally be combined with detection of one or more additional biomarkers for kidney response. Any suitable method in the art can be employed to measure the level of KRFs and KRPIs, including but not limited to the Preferred Technology described herein, kinase assays, immunoassays to detect and/or visualize the KRPI (e.g., Western blot, immunoprecipitation followed by sodium dodecyl sulfate polyacrylamide gel electrophoresis, immunocytochemistry, etc.). In cases where a KRPI has a known function, an assay for that function may be used to measure KRPI expression. In a further embodiment, a decreased abundance of mRNA including one or more KRPIs identified in Table VII or IX (or any combination of them) in a test sample relative to a control sample or a previously determined reference range indicates the presence of kidney response. In yet a further embodiment, an increased abundance of mRNA encoding one or more KRPIs identified in Table VIII or X (or any combination of them) in a test sample relative to a control sample or previously determined reference range indicates the presence of kidney response. Any suitable hybridization assay can be used to detect KRPI expression by detecting and/or visualizing mRNA encoding the KRPI (e.g., Northern assays, dot blots, in situ hybridization, etc.).

In another embodiment of the invention, labeled antibodies, derivatives and analogs thereof, which specifically bind to a KRPI can be used for diagnostic purposes to detect, diagnose, or monitor kidney response. Preferably, kidney response is detected in an animal, more preferably in a mammal and most preferably in a human.

Screening Assays

The invention provides methods for identifying agents (e.g., drug candidates or test compounds) that bind to a KRPI or have a stimulatory or inhibitory effect on the expression or activity of a KRPI. Preferably the KRPI is one of: KRPI-2, KRPI-8, KRPI-11, KRPI-13, KRPI-14, KRPI-15, KRPI-16, KRPI-19, KRPI-21, KRPI-23, KRPI-27, KRPI-28, KRPI-35, KRPI-40, KRPI-41, KRPI-42, KRPI-43, KRPI-45.1, KRPI-45.2, KRPI-57, KRPI-59, KRPI-60, KRPI-63, KRPI-70, KRPI-72, KRPI-73, KRPI-76, KRPI-84, KRPI-85, KRPI-86, KRPI-88, KRPI-90, KRPI-91, KRPI-98, KRPI-101, KRPI-104, KRPI-105, KRPI-113, KRPI-122, KRPI-123, KRPI-128, KRPI-131, KRPI-132, KRPI-134, KRPI-138, KRPI-139, KRPI-142, KRPI-143, KRPI-144, KRPI-149, KRPI-152, KRPI-153, KRPI-158, KRPI-159, KRPI-168, KRPI-170, KRPI-178, KRPI-179, KRPI-183, KRPI-184, KRPI-185, KRPI-186, KRPI-188, KRPI-189.1, KRPI-189.2, KRPI-192, KRPI-196, KRPI-202, KRPI-206, KRPI-208, KRPI-210, KRPI-219, KRPI-222, KRPI-229, KRPI-232, KRPI-235.1, KRPI-235.2, KRPI-236, KRPI-237, KRPI-240, KRPI-245, KRPI-247, KRPI-249, KRPI-250, KRPI-252, KRPI-253, KRPI-256, KRPI-257, KRPI-263, KRPI-267, KRPI-273, KRPI-278, KRPI-280, KRPI-282, KRPI-285, KRPI-286, KRPI-313, KRPI-314.1, KRPI-314.2, KRPI-327.1, KRPI-327.2, or KRPI-339. The invention also provides methods of identifying agents that bind to a KRPI-related polypeptide or a KRPI fusion protein or have a stimulatory or inhibitory effect on the expression or activity of a KRPI-related polypeptide or a KRPI fusion protein.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that do or do not interact with (i.e., bind to) a KRPI, a KRPI fragment (e.g. a functionally active fragment), a KRPI-related polypeptide, a fragment of a KRPI-related polypeptide, or a KRPI fusion protein are identified in a cell-based assay system. In accordance with this embodiment, cells expressing a KRPI, a fragment of a KRPI, a KRPI-related polypeptide, a fragment of a KRPI-related polypeptide, or a KRPI fusion protein are contacted with an agent, such as a drug candidate, or a control and the ability of the agent to interact with the KRPI is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the KRPI, fragment of the KRPI, KRPI-related polypeptide, a fragment of the KRPI-related polypeptide, or a KRPI fusion protein endogenously or be genetically engineered to express the KRPI, fragment of the KRPI, KRPI-related polypeptide, a fragment of the KRPI-related polypeptide, or a KRPI fusion protein. In certain instances, the KRPI, fragment of the KRPI, KRPI-related polypeptide, a fragment of the KRPI-related polypeptide, or a KRPI fusion protein or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a KRPI and and agent, such as a drug candidate. The ability of the candidate compound to interact directly or indirectly with a KRPI, a fragment of a KRPI, a KRPI-related polypeptide, a fragment of a KRPI-related polypeptide, or a KRPI fusion protein can be determined by methods known to those of skill in the art. For example, the interaction between a candidate compound and a KRPI, a fragment of a KRPI, a KRPI-related polypeptide, a fragment of a KRPI-related polypeptide, or a KRPI fusion protein can be determined by flow cytometry, a scintillation assay, immunoprecipitation or western blot analysis.

In another embodiment, agents that do or do not interact with (i.e., bind to) a KRPI, a KRPI fragment (e.g., a functionally active fragment) a KRPI-related polypeptide, a fragment of a KRPI-related polypeptide, or a KRPI fusion protein are identified in a cell-free assay system. In accordance with this embodiment, a native or recombinant KRPI or fragment thereof, or a native or recombinant KRPI-related polypeptide or fragment thereof, or a KRPI-fusion protein or fragment thereof, is contacted with an agent or a control and the ability of the agent to interact with the KRPI or KRPI-related polypeptide, or KRPI fusion protein is determined. If desired, this assay may be used to screen a plurality (e.g. a library) of agents. Preferably, the KRPI, KRPI fragment, KRPI-related polypeptide, a fragment of a KRPI-related polypeptide, or a KRPI-fusion protein is first immobilized, by, for example, contacting the KRPI, KRPI fragment, KRPI-related polypeptide, a fragment of a KRPI-related polypeptide, or a KRPI fusion protein with an immobilized antibody which specifically recognizes and binds it, or by contacting a purified preparation of the KRPI, KRPI fragment, KRPI-related polypeptide, fragment of a KRPI-related polypeptide, or a KRPI fusion protein with a surface designed to bind proteins. The KRPI, KRPI fragment, KRPI-related polypeptide, a fragment of a KRPI-related polypeptide, or a KRPI fusion protein may be partially or completely purified (e.g., partially or completely free of other polypeptides) or part of a cell lysate. Further, the KRPI, KRPI fragment, KRPI-related polypeptide, a fragment of a KRPI-related polypeptide may be a fusion protein comprising the KRPI or a biologically active portion thereof, or KRPI-related polypeptide and a domain such as glutathionine-S-transferase. Alternatively, the KRPI, KRPI fragment, KRPI-related polypeptide, fragment of a KRPI-related polypeptide or KRPI fusion protein can be biotinylated using techniques well known to those of skill in the art (e.g., biotinylation kit, Pierce Chemicals; Rockford, Ill.). The ability of the agent to interact with a KRPI, KRPI fragment, KRPI-related polypeptide, a fragment of a KRPI-related polypeptide, or a KRPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a KRPI or is responsible for the post-translational modification of a KRPI. In a primary screen, a plurality (e.g., a library) of agents e.g. drug candidates are contacted with cells that naturally or recombinantly express: (i) a KRPI, an isoform of a KRPI, a KRPI homolog a KRPI-related polypeptide, a KRPI fusion protein, or a biologically active fragment of any of the foregoing; and (ii) a protein that is responsible for processing of the KRPI, KRPI isoform, KRPI homolog, KRPI-related polypeptide, KRPI fusion protein, or fragment in order to identify compounds that modulate the production, degradation, or post-translational modification of the KRPI, KRPI isoform, KRPI homolog, KRPI-related polypeptide, KRPI fusion protein or fragment. If desired, agents identified in the primary screen can then be assayed in a secondary screen against cells naturally or recombinantly expressing the specific KRPI of interest. The ability of the agent to modulate the production, degradation or post-translational modification of a KRPI, isoform, homolog, KRPI-related polypeptide, or KRPI fusion protein can be determined by methods known to those of skill in the art, including without limitation, flow cytometry, a scintillation assay, immunoprecipitation and western blot analysis.

In another embodiment, agents that do or do not competitively interact with (i.e., bind to) a KRPI, KRPI fragment, KRPI-related polypeptide, a fragment of a KRPI-related polypeptide, or a KRPI fusion protein are identified in a competitive binding assay. In accordance with this embodiment, cells expressing a KRPI, KRPI fragment, KRPI-related polypeptide, a fragment of a KRPI-related polypeptide, or a KRPI fusion protein are contacted with an agent and a compound known to interact with the KRPI, KRPI fragment, KRPI-related polypeptide, a fragment of a KRPI-related polypeptide or a KRPI fusion protein; the ability of the agent to competitively interact with the KRPI, KRPI fragment, KRPI-related polypeptide, fragment of a KRPI-related polypeptide, or a KRPI fusion protein is then determined. Alternatively, agents that competitively interact with (i.e., bind to) a KRPI, KRPI fragment, KRPI-related polypeptide or fragment of a KRPI-related polypeptide are identified in a cell-free assay system by contacting a KRPI, KRPI fragment, KRPI-related polypeptide, fragment of a KRPI-related polypeptide, or a KRPI fusion protein with a candidate agent and a compound known to interact with the KRPI, KRPI-related polypeptide or KRPI fusion protein. As stated above, the ability of the candidate agent to interact with a KRPI, KRPI fragment, KRPI-related polypeptide, a fragment of a KRPI-related polypeptide, or a KRPI fusion protein can be determined by methods known to those of skill in the art. These assays, whether cell-based or cell-free, can be used to screen a plurality (e.g., a library) of candidate agents.

In another embodiment, agents that do or do not modulate (i.e., upregulate or downregulate) the expression of a KRPI, or a KRPI-related polypeptide are identified by contacting cells (e.g., cells of prokaryotic origin or eukaryotic origin) expressing the KRPI, or KRPI-related polypeptide with a candidate agent or a control (e.g., phosphate buffered saline (PBS)) and determining the expression of the KRPI, KRPI-related polypeptide, or KRPI fusion protein, mRNA encoding the KRPI, or mRNA encoding the KRPI-related polypeptide. The level of expression of a selected KRPI, KRPI-related polypeptide, mRNA encoding the KRPI, or mRNA encoding the KRPI-related polypeptide in the presence of the candidate agent is compared to the level of expression of the KRPI, KRPI-related polypeptide, mRNA encoding the KRPI, or mRNA encoding the KRPI-related polypeptide in the absence of the candidate agent (e.g., in the presence of a control). The candidate agent can then be identified as a modulator of the expression of the KRPI, or a KRPI-related polypeptide based on this comparison. For example, when expression of the KRPI or mRNA is significantly greater in the presence of the candidate agent than in its absence, the candidate agent is identified as a stimulator of expression of the KRPI or mRNA. Alternatively, when expression of the KRPI or mRNA is significantly less in the presence of the candidate compound than in its absence, the candidate compound is identified as an inhibitor of the expression of the KRPI or mRNA. The level of expression of a KRPI or the mRNA that encodes it can be determined by methods known to those of skill in the art. For example, mRNA expression can be assessed by Northern blot analysis or RT-PCR, and protein levels can be assessed by western blot analysis.

In another embodiment, agents that do or do not modulate the activity of a KRPI, or a KRPI-related polypeptide are identified by contacting a preparation containing the KRPI or KRPI-related polypeptide, or cells (e.g., prokaryotic or eukaryotic cells) expressing the KRPI or KRPI-related polypeptide with a test agent or a control and determining the ability of the test agent to modulate (e.g., stimulate or inhibit) the activity of the KRPI or KRPI-related polypeptide. The activity of a KRPI or a KRPI-related polypeptide can be assessed by detecting induction of a cellular signal transduction pathway of the KRPI or KRPI-related polypeptide (e.g., intracellular Ca2+, diacylglycerol, IP3, etc.), detecting catalytic or enzymatic activity of the target on a suitable substrate, detecting the induction of a reporter gene (e.g., a regulatory element that is responsive to a KRPI or a KRPI-related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference). The candidate agent can then be identified as a modulator of the activity of a KRPI or KRPI-related polypeptide by comparing the effects of the candidate agent to the control. Suitable control compounds include phosphate buffered saline (PBS) and normal saline (NS).

In another embodiment, agents that do or do not modulate (i.e., upregulate or downregulate) the expression, activity or both the expression and activity of a KRPI or KRPI-related polypeptide are identified in an animal model. Examples of suitable animals include, but are not limited to, mice, rats, rabbits, monkeys, guinea pigs, dogs and cats. Preferably, the animal used represent a model of kidney response. In accordance with this embodiment, the test agent or a control is administered (e.g., orally, rectally or parenterally such as intraperitoneally or intravenously) to a suitable animal and the effect on the expression, activity or both expression and activity of the KRPI or KRPI-related polypeptide is determined. Changes in the expression of a KRPI or KRPI-related polypeptide can be assessed by the methods outlined above. As the method for screening drug candiaites for their potential to induce a kidney response, the agents tested are advantageously agents which will be administered systemically, e.g. intravenously, since it is such agents that are most likely to induce an unwanted kidney response.

In yet another embodiment, a KRPI or KRPI-related polypeptide is used as a “bait protein” in a two-hybrid assay or three hybrid assay to identify other proteins that bind to or interact with a KRPI or KRPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the KRPIs of the invention as, for example, upstream or downstream elements of a signaling pathway involving the KRPIs of the invention.

More particularly, in one aspect, the invention provides methods for the identification of agents which will not have an effect on the expression or activity of a KRPI, KRPI-related polypeptide or KRPI fusion protein, and as such will not induce a kidney response. When such agents are drug candidiates they can be progressed into development with a greater level of confidence that they will not produce unwanted kidney responses when administered clinically.

This aspect of the invention allows for toxicity screening to be carried out at a much earlier stage. In particular, it can show whether an agent will or will not induce kidney response. In relation to the screening of agents for their potential to induce an unwanted kidney response, The term “agent” is used herein to describe a wide variety of physical, chemical or biological factors. For example, physical agents include, without limitation, the diet of a subject, a change in temperature or humidity, exposure to ultraviolet radiation and the like. Biological and chemical agents include exogenous factors such as pharmaceutical compounds (including candidate compounds and test compounds), toxic compounds, proteins, peptides, chemical compositions, natural pathogens, such as microbial agents including bacteria, viruses and lower eukaryotic cells such as fungi, yeast and simple multicellular organisms, as well as endogenous factors which occur naturally in the body, including, without limitation, hormones, enzymes, receptors, ligands and the like, which may or may not be recombinant.

This invention further provides novel agents identified by the above-described screening assays and uses thereof for treatments as described herein. Therapeutic Uses of KRPIs

The invention provides for treatment or prevention of various diseases and disorders by administration of a therapeutic compound. Such compounds include but are not limited to: KRPIs, KRPI analogs, KRPI-related polypeptides and derivatives (including fragments) thereof; antibodies to the foregoing; nucleic acids encoding KRPIs, KRPI analogs, KRPI-related polypeptides and fragments thereof; antisense nucleic acids to a gene encoding a KRPI or KRPI-related polypeptide; and modulator (e.g., agonists and antagonists) of a gene encoding a KRPI or KRPI-related polypeptide. An important feature of the present invention is the identification of genes encoding KRPIs involved in kidney response. Kidney response can be treated (e.g. to ameliorate symptoms or to retard onset or progression) or prevented by administration of a therapeutic compound that promotes function or expression of one or more KRPIs that are decreased in the blood or kidney tissue of subjects having kidney response, or by administration of a therapeutic compound that reduces function or expression of one or more KRPIs that are increased in the blood or kidney tissue of subjects having kidney response.

In one embodiment, one or more antibodies each specifically binding to a KRPI are administered alone or in combination with one or more additional therapeutic compounds or treatments.

Preferably, a biological product such as an antibody is allogeneic to the subject to which it is administered. In a preferred embodiment, a human KRPI or a human KRPI-related polypeptide, a nucleotide sequence encoding a human KRPI or a human KRPI-related polypeptide, or an antibody to a human KRPI or a human KRPI-related polypeptide, is administered to a human subject for therapy (e.g. to ameliorate symptoms or to retard onset or progression) or prophylaxis.

Treatment and Prevention of Kidney Response

Kidney response is treated or prevented by administration to a subject suspected of having or known to have kidney response or to be at risk of developing kidney response of a compound that modulates (i.e., increases or decreases) the level or activity (i.e., function) of one or more KRPIs—or the level of one or more KRFs—that are differentially present in the blood or kidney tissue of subjects having kidney response compared with blood or kidney tissue of subjects free from kidney response. In one embodiment, kidney response is treated or prevented by administering to a subject suspected of having or known to have kidney response or to be at risk of developing kidney response a compound that upregulates (i.e., increases) the level or activity (i.e., function) of one or more KRPIs—or the level of one or more KRFs—that are decreased in the blood of subjects having kidney response. In another embodiment, a compound is administered that upregulates the level or activity (i.e., function) of one or more KRPIs—or the level of one or more KRFs—that are increased in the blood of subjects having kidney response. Examples of such a compound include but are not limited to: KRPIs, KRPI fragments and KRPI-related polypeptides; nucleic acids encoding a KRPI, a KRPI fragment and a KRPI-related polypeptide (e.g., for use in gene therapy); and, for those KRPIs or KRPI-related polypeptides with enzymatic activity, compounds or molecules known to modulate that enzymatic activity. Other compounds that can be used, e.g., KRPI agonists, can be identified using in vitro assays.

Kidney response is also treated or prevented by administration to a subject suspected of having or known to have kidney response or to be at risk of developing kidney response of a compound that downregulates the level or activity of one or more KRPIs—or the level of one or more KRFs—that are increased in the blood or kidney tissue of subjects having kidney response. In another embodiment, a compound is administered that downregulates the level or activity of one or more KRPIs—or the level of one or more KRFs—that are decreased in the blood or kidney tissue of subjects having kidney response. Examples of such a compound include, but are not limited to, KRPI antisense oligonucleotides, ribozymes, antibodies directed against KRPIs, and compounds that inhibit the enzymatic activity of a KRPI. Other useful compounds e.g., KRPI antagonists and small molecule KRPI antagonists, can be identified using in vitro assays.

In a preferred embodiment, therapy or prophylaxis is tailored to the needs of an individual subject. Thus, in specific embodiments, compounds that promote the level or function of one or more KRPIs, or the level of one or more KRFs, are therapeutically or prophylactically administered to a subject suspected of having or known to have kidney response, in whom the levels or functions of said one or more KRPIs, or levels of said one or more KRFs, are absent or are decreased relative to a control or normal reference range. In further embodiments, compounds that promote the level or function of one or more KRPIs, or the level of one or more KRFs, are therapeutically or prophylactically administered to a subject suspected of having or known to have kidney response in whom the levels or functions of said one or more KRPIs, or levels of said one or more KRFs, are increased relative to a control or to a reference range. In further embodiments, compounds that decrease the level or function of one or more KRPIs, or the level of one or more KRFs, are therapeutically or prophylactically administered to a subject suspected of having or known to have kidney response in whom the levels or functions of said one or more KRPIs, or levels of said one or more KRFs, are increased relative to a control or to a reference range. In further embodiments, compounds that decrease the level or function of one or more KRPIs, or the level of one or more KRFs, are therapeutically or prophylactically administered to a subject suspected of having or known to have kidney response in whom the levels or functions of said one or more KRPIs, or levels of said one or more KRFs, are decreased relative to a control or to a reference range. The change in KRPI function or level, or KRF level, due to the administration of such compounds can be readily detected, e.g., by obtaining a sample (e.g., a sample of blood, blood or urine or a tissue sample such as biopsy tissue) and assaying in vitro the levels of said KRFs or the levels or activities of said KRPIs, or the levels of mRNAs encoding said KRPIs or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the kidney response KRPI or KRF profile towards normal.

Gene Therapy

In a specific embodiment, nucleic acids comprising a sequence encoding a KRPI, a KRPI fragment, KRPI-related polypeptide or fragment of a KRPI-related polypeptide, are administered to promote KRPI function by way of gene therapy. Gene therapy refers to administration to a subject of an expressed or expressible nucleic acid. In this embodiment, the nucleic acid produces its encoded polypeptide that mediates a therapeutic effect by promoting KRPI function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the compound comprises a nucleic acid encoding a KRPI or fragment or chimeric protein thereof, said nucleic acid being part of an expression vector that expresses a KRPI or fragment or chimeric protein thereof in a suitable host. In particular, such a nucleic acid has a promoter operably linked to the KRPI coding region, said promoter being inducible or constitutive (and, optionally, tissue-specific). In another particular embodiment, a nucleic acid molecule is used in which the KRPI coding sequences and any other desired sequences are flanked by regions that promote homologous recombination at a desired site in the genome, thus providing for intrachromosomal expression of the KRPI nucleic acid (Koller and Snuthies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject; this approach is known as ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO92/22635 dated Dec, 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains a nucleic acid encoding a KRPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the KRPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a KRPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of genetherapy comprises an inducible promoter operably linked to the coding region, such that expression of the nucleic acid is controllable by controlling the presence or absence of the appropriate inducer of transcription.

Direct injection of a DNA coding for a KRPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a KRPI and (b) a promoter are injected into a subject to elicit an immune response to the KRPI.

Inhibition of KRPIs to Treat Kidney Response

In one embodiment of the invention, kidney response is treated or prevented by administration of a compound that antagonizes (inhibits) the level(s) and/or function(s) of one or more KRPIs which are elevated in the blood or kidney tissue of subjects having kidney response as compared with blood or kidney tissue of subjects free from kidney response. Compounds useful for this purpose include but are not limited to anti-KRPI antibodies (and fragments and derivatives containing the binding region thereof), KRPI antisense or ribozyme nucleic acids, and nucleic acids encoding dysfunctional KRPIs that are used to “knockout” endogenous KRPI function by homologous recombination (see, e.g., Capecchi, 1989, Science 244:1288-1292). Other compounds that inhibit KRPI function can be identified by use of known in vitro assays, e.g., assays for the ability of a test compound to inhibit binding of a KRPI to another protein or a binding partner, or to inhibit a known KRPI function. Preferably such inhibition is assayed in vitro or in cell culture, but genetic assays may also be employed. The Preferred Technology can also be used to detect levels of the KRPI before and after the administration of the compound. Preferably, suitable in vitro or in vivo assays are utilized to determine the effect of a specific compound and whether its administration is indicated for treatment of the affected tissue, as described in more detail below.

In a specific embodiment, a compound that inhibits a KRPI function is administered therapeutically or prophylactically to a subject in whom an increased blood level or functional activity of the KRPI (e.g., greater than the normal level or desired level) is detected as compared with blood or kidney tissue of subjects free from kidney response or a predetermined reference range. Methods standard in the art can be employed to measure the increase in a KRPI level or function, as outlined above. Preferred KRPI inhibitor compositions include small molecules, i.e., molecules of 1000 daltons or less. Such small molecules can be identified by the screening methods described herein.

Antisense Regulation of KRPIs

In a specific embodiment, KRPI expression is inhibited by use of KRPI antisense nucleic acids. The present invention provides the therapeutic or prophylactic use of nucleic acids comprising at least six nucleotides that are antisense to a gene or cDNA encoding a KRPI or a portion thereof. As used herein, a KRPI “antisense” nucleic acid refers to a nucleic acid capable of hybridizing by virtue of some sequence complementarity to a portion of an RNA (preferably mRNA) encoding a KRPI. The antisense nucleic acid may be complementary to a coding and/or noncoding region of an mRNA encoding a KRPI. Such antisense nucleic acids have utility as compounds that inhibit KRPI expression, and can be used in the treatment or prevention of kidney response.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the KRPI antisense nucleic acids of the invention in a pharmaceutically acceptable carrier, as described infra.

In another embodiment, the invention provides methods for inhibiting the expression of a KRPI nucleic acid sequence in a prokaryotic or eukaryotic cell comprising providing the cell with an effective amount of a composition comprising a KRPI antisense nucleic acid of the invention.

KRPI antisense nucleic acids and their uses are described in detail below.

KRPI Antisense Nucleic Acids

The KRPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a KRPI antisense oligonucleotide is provided, preferably of single-stranded DNA. The oligonucleotide may be modified at any position on its structure with substituents generally known in the art.

The KRPI antisense oligonucleotide may comprise at least one of the following modified base moieties: 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylanunomethyluracil, 5-methoxyaminomethyl-2-thiouracil, beta-D-mannosylqueosine, 5N-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-dianunopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphoniate, an alkyl phosphotriester, a formacetal, or an analog of formacetal.

In yet another embodiment, the oligonucleotide is an α-anomeric oligonucleotide. An a-anomeric oligonucleotide forms specific double-stranded hybrids with complementary RNA in which, contrary to the usual (3-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In a specific embodiment, the KRPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the KRPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the KRPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a KRPI, preferably a human gene encoding a KRPI. However, absolute complementarity, although preferred, is not required. A sequence “complementary to at least a portion of an RNA,” as referred to herein, means a sequence having sufficient complementarity to be able to hybridize under stringent conditions (e.g., highly stringent conditions comprising hybridization in 7% sodium dodecyl sulfate (SDS), 1 mM EDTA at 65_C and washing in 0.1×SSC/0.1% SDS at 68° C., or moderately stringent conditions comprising washing in 0.2×SSC/0.1% SDS at 42° C.) with the RNA, forming a stable duplex; in the case of double-stranded KRPI antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding a KRPI it may contain and still form a stable duplex (or triplex, as the case may be). One skilled in the art can ascertain a tolerable degree of mismatch by use of standard procedures to determine the melting point of the hybridized complex.

Therapeutic Use of KRPI Antisense Nucleic Acids

The KRPI antisense nucleic acids can be used to treat or prevent kidney response when the target KRPI is overexpressed in the blood of subjects suspected of having or suffering from kidney response. In a preferred embodiment, a single-stranded DNA antisense KRPI oligonucleotide is used.

Cell types which express or overexpress RNA encoding a KRPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with a KRPI-specific nucleic acid (e.g., by Northern hybridization, dot blot hybridization, in situ hybridization), observing the ability of RNA from the cell type to be translated in vitro into a KRPI, immunoassay, etc. In a preferred aspect, primary tissue from a subject can be assayed for KRPI expression prior to treatment, e.g., by immunocytochemistry or in situ hybridization.

Pharmaceutical compositions of the invention, comprising an effective amount of a KRPI antisense nucleic acid in a pharmaceutically acceptable carrier, can be administered to a subject having kidney response.

The amount of KRPI antisense nucleic acid which will be effective in the treatment of kidney response can be determined by standard clinical techniques.

In a specific embodiment, pharmaceutical compositions comprising one or more KRPI antisense nucleic acids are administered via liposomes, microparticles, or microcapsules. In various embodiments of the invention, such compositions may be used to achieve sustained release of the KRPI antisense nucleic acids.

Inhibitory Ribozyme and Triple Helix Approaches

In another embodiment, symptoms of kidney response may be ameliorated by decreasing the level of a KRPI or KRPI activity by using gene sequences encoding the KRPI in conjunction with well-known gene “knock-out,” ribozyme or triple helix methods to decrease gene expression of a KRPI. In this approach ribozyme or triple helix molecules are used to modulate the activity, expression or synthesis of the gene encoding the KRPI, and thus to ameliorate the symptoms of kidney response. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding a KRPI can be used to prevent translation of target gene mRNA and, therefore, expression of the gene product. (See, e.g., PCT International Publication WO09/11364, published Oct. 4, 1990; Sarver et al., 1990, Science 247:1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding a KRPI, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334,585-591, each of which is incorporated herein by reference in its entirety.

Preferably the ribozyme is engineered so that the cleavage recognition site is located near the 5′ end of the mRNA encoding the KRPI, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224,574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47,207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the KRPI.

As in the antisense approach, the ribozymes can be composed of modified oligonucleotides (e.g., for improved stability, targeting, etc.) and should be delivered to cells that express the KRPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the KRPI and inhibit translation. Because ribozymes, unlike antisense molecules, are catalytic, a lower intracellular concentration is required for efficacy.

Endogenous KRPI expression can also be reduced by inactivating or “knocking out” the gene encoding the KRPI, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra et al., 1989, Nature 342:435-438, each of which is incorporated by reference herein in its entirety). For example, a mutant gene encoding a non-functional KRPI (or a completely unrelated DNA sequence) flanked by DNA homologous to the endogenous gene (either the coding regions or regulatory regions of the gene encoding the KRPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, the endogenous expression of a gene encoding a KRPI can be reduced by targeting deoxyribonucleotide sequences complementary to the regulatory region of the gene (i.e., the gene promoter and/or enhancers) to form triple helical structures that prevent transcription of the gene encoding the KRPI in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of a KRPI that the situation may arise wherein the concentration of KRPI present may be lower than is necessary for a normal phenotype. In such cases, to ensure that substantially normal levels of activity of a gene encoding a KRPI are maintained, gene therapy may be used to introduce into cells nucleic acid molecules that encode and express the KRPI that exhibit normal gene activity and that do not contain sequences susceptible to whatever antisense, ribozyme, or triple helix treatments are being utilized. Alternatively, in instances whereby the gene encodes an extracellular protein, normal KRPI can be co-administered in order to maintain the requisite level of KRPI activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Assays for Therapeutic or Prophylactic Compounds

The present invention also provides assays for use in drug discovery in order to identify or verify the efficacy of compounds for treatment or prevention of kidney response. Test compounds can be assayed for their ability to restore KRF or KRPI levels in a subject having kidney response towards levels found in subjects free from kidney response or to produce similar changes in experimental animal models of kidney response. Compounds able to restore KRF or KRPI levels in a subject having kidney response towards levels found in subjects free from kidney response or to produce similar changes in experimental animal models of kidney response can be used as lead compounds for further drug discovery, or used therapeutically. KRF and KRPI expression can be assayed by the Preferred Technology, immunoassays, gel electrophoresis followed by visualization, detection of KRPI activity, or any other method taught herein or known to those skilled in the art. Such assays can be used to screen candidate drugs, in clinical monitoring or in drug development, where abundance of a KRF or KRPI can serve as a surrogate marker for clinical disease.

Preferably the KRPI is selected from one of: KRPI-2, KRPI-8, KRPI-11, KRPI-13, KRPI-14, KRPI-15, KRPI-16, KRPI-19, KRPI-21, KRPI-23, KRPI-27, KRPI-28, KRPI-35, KRPI-40, KRPI-41, KRPI-42, KRPI-43, KRPI-45.1, KRPI-45.2, KRPI-57, KRPI-59, KRPI-60, KRPI-63, KRPI-70, KRPI-72, KRPI-73, KRPI-76, KRPI-84, KRPI-85, KRPI-86, KRPI-88, KRPI-90, KRPI-91, KRPI-98, KRPI-101, KRPI-104, KRPI-105, KRPI-113, KRPI-122, KRPI-123, KRPI-128, KRPI-131, KRPI-132, KRPI-134, KRPI-138, KRPI-139, KRPI-142, KRPI-143, KRPI-144, KRPI-149, KRPI-152, KRPI-153, KRPI-158, KRPI-159, KRPI-168, KRPI-170, KRPI-178, KRPI-179, KRPI-183, KRPI-184, KRPI-185, KRPI-186, KRPI-188, KRPI-189.1, KRPI-189.2, KRPI-192, KRPI-196, KRPI-202, KRPI-206, KRPI-208, KRPI-210, KRPI-219, KRPI-222, KRPI-229, KRPI-232, KRPI-235.1, KRPI-235.2, KRPI-236, KRPI-237, KRPI-240, KRPI-245, KRPI-247, KRPI-249, KRPI-250, KRPI-252, KRPI-253, KRPI-256, KRPI-257, KRPI-263, KRPI-267, KRPI-273, KRPI-278, KRPI-280, KRPI-282, KRPI-285, KRPI-286, KRPI-313, KRPI-314.1, KRPI-314.2, KRPI-327.1, KRPI-327.2, or KRPI-339.

In various specific embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used It is also apparent to the skilled artisan that, based upon the present disclosure, transgenic animals can be produced with “knock-out” mutations of the gene or genes encoding one or more KRPIs. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of a KRPI are identified in non-human animals (e.g., mice, rats, monkeys, rabbits, and guinea pigs), preferably non-human animal models for kidney response, expressing the KRPI. In accordance with this embodiment, a test compound or a control compound is administered to the animals, and the effect of the test compound on expression of one or more KRPIs is determined. A test compound that alters the expression of a KRPI (or a plurality of KRPIs) can be identified by comparing the level of the selected KRPI or KRPIs (or mRNA(s) encoding the same) in an animal or group of animals treated with a test compound with the level of the KRPI(s) or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of a KRPI or a biologically active portion thereof are identified in non-human animals (e.g., mice, rats, monkeys, rabbits, and guinea pigs), preferably non-human animal models for kidney response, expressing the KRPI. In accordance with this embodiment, a test compound or a control compound is administered to the animals, and the effect of a test compound on the activity of a KRPI is determined. A test compound that alters the activity of a KIRPI (or a plurality of KRPIs) can be identified by assaying animals treated with a control compound and animals treated with the test compound. The activity of the KRPI can be assessed by detecting induction of a cellular second messenger of the KRPI (e.g., intracellular Ca2+, diacylglycerol, IP3, etc.), detecting catalytic or enzymatic activity of the KRPI or binding partner thereof, detecting the induction of a reporter gene (e.g., a regulatory element that is responsive to a KRPI of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of a KRPI (see, e.g., U.S. Pat. No. 5,401,639, which is incorporated herein by reference).

In yet another embodiment, test compounds that modulate the level or expression of a KRPI (or plurality of KRPIs) are identified in human subjects having kidney response, preferably those having kidney response and most preferably those having severe kidney response. In accordance with this embodiment, a test compound or a control compound is administered to the human subject, and the effect of a test compound on KRPI expression is determined by analyzing the expression of the KRPI or the mRNA encoding the same in a biological sample (e.g., blood, serum, plasma, or urine). A test compound that alters the expression of a KRPI can be identified by comparing the level of the KRPI or mRNA encoding the same in a subject or group of subjects treated with a control compound to that in a subject or group of subjects treated with a test compound. Alternatively, alterations in the expression of a KRPI can be identified by comparing the level of the KRPI or mRNA encoding the same in a subject or group of subjects before and after the administration of a test compound. Techniques known to those of skill in the art can be used to obtain the biological sample and analyze the mRNA or protein expression. For example, the Preferred Technology described herein can be used to assess changes in the level of a KRPI.

In another embodiment, test compounds that modulate the activity of a KRPI (or plurality of KRPIs) are identified in human subjects having kidney response, preferably those having kidney response and most preferably those with severe kidney response. In this embodiment, a test compound or a control compound is administered to the human subject, and the effect of a test compound on the activity of a KRPI is determined. A test compound that alters the activity of a KRPI can be identified by comparing biological samples from subjects treated with a control compound to samples from subjects treated with the test compound. Alternatively, alterations in the activity of a KRPI can be identified by comparing the activity of a KRPI in a subject or group of subjects before and after the administration of a test compound. The activity of the KRPI can be assessed by detecting in a biological sample (e.g., blood, serum, plasma, or urine) induction of a cellular signal transduction pathway of the KRPI (e.g., intracellular Ca2+, diacylglycerol, IP3, etc.), catalytic or enzymatic activity of the KRPI or a binding partner thereof, or a cellular response, for example, cellular differentiation, or cell proliferation. Techniques known to those of skill in the art can be used to detect changes in the induction of a second messenger of a KRPI or changes in a cellular response. For example, RT-PCR can be used to detect changes in the induction of a cellular second messenger.

In a preferred embodiment, a test compound that changes the level or expression of a KRPI towards levels detected in control subjects (e.g., humans free from kidney response) is selected for further testing or therapeutic use. In another preferred embodiment, a test compound that changes the activity of a KRPI towards the activity found in control subjects (e.g., humans free from kidney response) is selected for further testing or therapeutic use.

In another embodiment, test compounds that reduce the severity of one or more symptoms associated with kidney response are identified in human subjects having kidney response, preferably subjects having kidney response and most preferably subjects with severe kidney response. In accordance with this embodiment, a test compound or a control compound is administered to the subjects, and the effect of a test compound on one or more symptoms of kidney response is determined. A test compound that reduces one or more symptoms can be identified by comparing the subjects treated with a control compound to the subjects treated with the test compound. Techniques known to physicians familiar with kidney response can be used to determine whether a test compound reduces one or more symptoms associated with kidney response.

In a preferred embodiment, a test compound that reduces the severity of one or more symptoms associated with kidney response in a human having kidney response is selected for further testing or therapeutic use.

Therapeutic and Prophylactic Compositions and their Use

The invention provides methods of treatment (and prophylaxis) comprising administering to a subject an effective amount of a compound of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into blood or at the site (or former site) of kidney response or kidney tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the kidney, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of kidney response can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLE

### Identification of Proteins Differentially Expressed in the Blood and Kidney Tissue in Kidney Response

Gentamicin, a known kidney toxin, was used to treat rats at a range of doses known to produce varying degrees of bistopathologically evident kidney response. Groups of rats were treated with Gentamicin at the following dose levels: 0.1, 1.0, 10, 40 or 60 mg/kg/day. The rat groups included 10 male rats per treated group, and 20 male rats in the untreated (control) group. Blood samples from rats treated at 40 mg/kg/day after 8 days were taken for proteome analysis, and kidney cortex tissue samples from rats treated at 0.1, 1.0, 10, and 40 mg/kg/day after 8 and 22 days for each group were taken for proteome analysis. Kidney cortex tissue samples were also prepared for histologic examination according to standard tissue preparation protocols.

Clinical and Histologic Results

At 60 mg/kg/day there were changes in urine biochemistry parameters (raised NAG (N-acetyl-beta-D-glucosaminidase), GGT (gamma-glutamyl-transpeptidase) and urine volume, depressed specific gravity), consistent with kidney damage. Histologic examination revealed that there was single cell necrosis and loss of brush border of the proximal convoluted tubule epithelium of the kidney at 60 mg/kg. Seven days after the withdrawal of treatment, there was evidence of cortical tubular regeneration consistent with on going lesions between 8 and 22 days.

Using the following procedure, proteins in blood and kidney tissue samples from control animals and animals having kidney response were separated by isoelectric focusing followed by SDS-PAGE and analyzed. Parts 6.1.1 to 6.1.14 (inclusive) of the procedure set forth are hereby designated as the ‘Reference Protocol’

Materials and Methods

Sample Preparation

Kidney Sample Preparation

At the time of necropsy, a portion of tissue from the kidney corticomedullary region was removed to a conical tube and quick-frozen in liquid nitrogen. Approximately 10 mg of the kidney tissue was transferred to a chilled potter homogeniser mortar containing 10 μl of the protease inhibitor solution (Sigma P2714).

1.5 ml of the 2D sample buffer solution was added and the tissue was homogenised thoroughly on ice. The resulting supernatant was transferred to a labelled tube and frozen in liquid nitrogen.

Blood Sample Preparation

Approximately 2 ml of fresh venous blood was collected in pre-labelled EDTA collection tubes, (yields 0.8-1 ml plasma). The sample was mixed thoroughly and gently. The samples were then centrifuged, as soon as possible, after collection for 10 minutes exactly at 1500×g at 4° C. This results in the separation of the blood into two layers. The top layer (the plasma layer) was drawn off and added to another prelabelled tube containing protease inhibitor solution (Sigma P2714) (150 μl protease inhibitor solution/ml plasma). The contents were then mixed by gentle vortexing. The samples were then snap frozen and stored at −70° C.

A protein assay (Pierce BCA Cat # 23225) was performed on each sample as received. Prior to protein separation, each plasma sample was processed for selective depletion of certain proteins, in order to enhance and simplify protein separation and facilitate analysis by removing proteins that may interfere with or limit analysis of proteins of interest. See International Patent Patent Publication No. WO 99/63351, which is incorporated by reference in its entirety, with particular reference to pages 3 and 6.

Removal of albumin, haptoglobin, transferrin and immunoglobin G (IgG) from plasma (“plasma depletion”) was achieved by an affinity chromatography purification step in which the sample was passed through a series of “Hi-Trap” columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No.21013). The eluate containing unbound material was collected in fractions which were pooled and desalted/concentrated by centrifugal ultrafiltration. The sample was recovered in 2D Sample Buffer (see below) containing a cocktail of protease inhibitors (Sigma P2714) and stored at −70° C. to await further analysis by 2D PAGE.

Sample Preparation for 2D Analysis

An aliquot of the stored sample containing 300 microg of protein was prepared for 2D analysis by adding Resolytes 3.5-10 (BDH 443382x) to 2% (v/v), as well as a trace of Bromophenol Blue and further 2D Sample Buffer in a final volume of 370 microl.

2D Sample Buffer:

8M urea (BDH 452043w)

2M thiourea (Fluka 88810)

4% CHAPS (Sigma C3023)

65 mM dithiotheitol (DTT)

This mixture was vortexed, and centrifuged at 13000 rpm for 5 mins at 15° C., and the supernatant was analyzed by isoelectric focusing.

Isoelectric Focusing

Isoelectric focusing (IEF), was performed using the Immobiline7 DryStrip Kit (Pharmacia BioTech), following the procedure described in the manufacturer's instructions, see Instructions for Immobiline7 DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 501 of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to 5W. The temperature was held at 20° C. throughout the run.

Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of g-methacryl-oxypropyltrimethoxysilane in ethanol (BindSilaneJ; Pharmacia Cat. # 17-1330-01). The front plate was treated with (RepelSilaneJ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored at 4° C. in sealed polyethylene bags with 6 ml of gel buffer, and were used within 4 weeks.

SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2^{nd} D gel was filled with the agarose solution, and the equilibrated strip was placed into the agarose, and tapped gently with a palette knife until the gel was intimately in contact with the 2^{nd} D gel. The gels were placed in the 2^{nd} D running tank, as described by Amess et al., 1995, Electrophoresis 16: 1255-1267 (incorporated herein by reference in its entirety). The tank was filled with running buffer (as above) until the level of the buffer was just higher than the top of the region of the 2^{nd} D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016×), 50% (v/v) water (MilliQ-Millipore), which was continuously circulated over the gels. After an overnight incubation, the fixative was drained from the tank, and the gels were primed by immersionin 7.5% (v/v) acetic acid, 0.05% (w/v) SDS, 92.5% (v/v) water for 30 nuns. The priming solution was then drained, and the gels were stained by complete immersion for 4 hours in a staining solution of Pyridinium, 4-[2-[4-(dipentylamino)-2-trifluoromethylphenyl] ethenyl]-1-(sulfobutyl)-, inner salt, prepared by diluting a stock solution of this dye (2 mg/ml in DMSO) in 7.5% (v/v) aqueous acetic acid to give a final concentration of 1.2 mg/l; the staining solution was vacuum filtered through a 0.4 μm filter (Duropore) before use.

Imaging of the Gel

A computer-readable output was produced by imaging the fluorescently stained gels with the Apollo 2 scanner (Oxford Glycosciences, Oxford, UK) described in section 5.1, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

Digital Analysis of the Data

The data were processed as described in U.S. Pat. No. 6,064,754 as in the experimental protocol (incorporated herein by reference), and as set forth more particularly below.

The output from the scanner was first processed using the MELANIE7 II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in (IF format. Features were detected using the following parameters:

Smooths=1

Laplacian threshold 100

Partials threshold 50

Saturation=100

Peakedness=0

Minimum Perimeter=10

Assignment of pI and MW Values

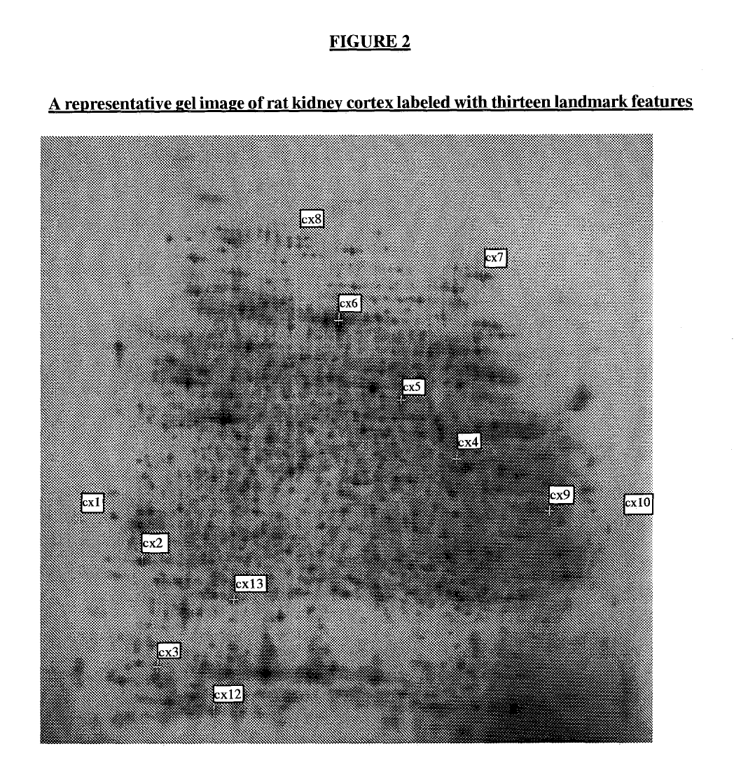

Landmark identification was used to determine the pI and MW of features detected in the images. Twelve landmark features, designated cx1, cx2, cx3, cx4, cx5, cx6, cx7, cx8, cx9, cx10, cx12, and cx13, were identified in a standard kidney cortex tissue image. These landmark features are identified in FIG. 2 and were assigned the pI and/or MW values identified in Table XIV.

TABLE XIV
Landmark Features of Kidney Cortex Tissue Used In This Study

| Name | pI | MW (Da) |
|---|---|---|
| cx1 | 4.28 | −1 |
| cx2 | 4.62 | 24092 |
| cx3 | 4.72 | 14017 |
| cx4 | 7.29 | 38372 |
| cx5 | 6.51 | 48595 |
| cx6 | 6.01 | 66684 |
| cx7 | −1 | 82005 |
| cx8 | 5.73 | 111549 |
| cx9 | 8.76 | 31342 |
| cx10 | 10 | −1 |
| cx12 | 5.14 | 10892 |
| cx13 | 5.32 | 20135 |

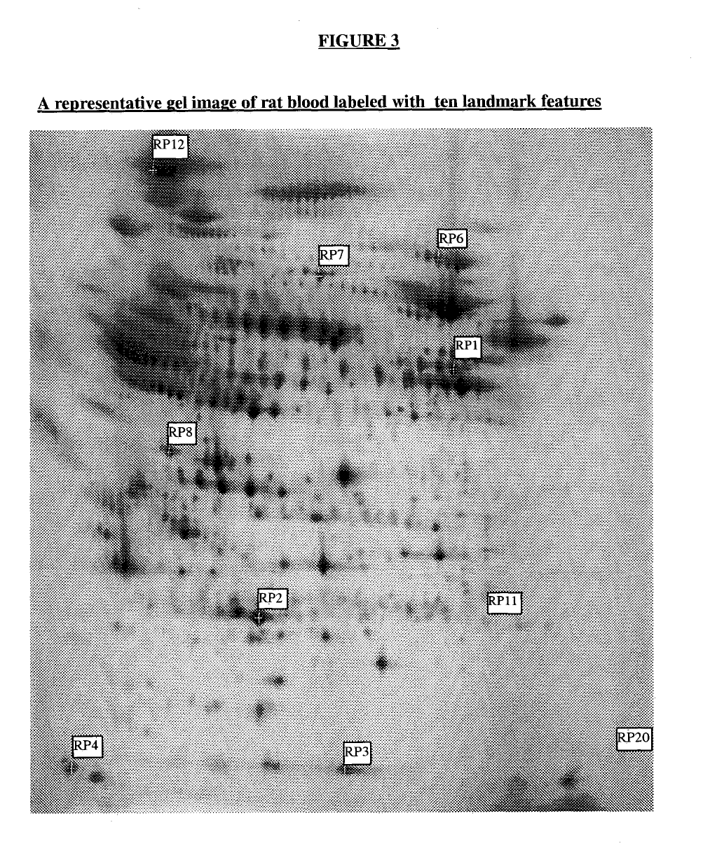

Ten landmark features, designated RP1, RP2, RP3, RP4, RP6, RP7, RP8, RP11, RP12, and RP20, were identified in a standard blood image obtained from a pooled sample. These landmark features are identified in FIG. 3 and were assigned the pI and/or MW values identified in Table XV.

TABLE XV
Landmark Features of Blood Used In This Study

| Name | pI | MW (Da) |
|---|---|---|
| RP1 | 7.25 | 55922 |
| RP2 | 5.55 | 22781 |
| RP3 | 6.12 | 11903 |
| RP4 | 4.41 | −1 |
| RP6 | −1 | 104343 |
| RP7 | 5.90 | 95547 |
| RP8 | 4.97 | 4149 |
| RP11 | 8 | −1 |
| RP12 | −1 | 150000 |
| RP20 | 10 | −1 |

As many of these landmarks as possible were identified in each gel image of the dataset. Each feature in the study gels was then assigned a pI value by linear interpolation or extrapolation (using the MELANIE7-II software) to the two nearest landmarks, and was assigned a MW value by linear interpolation or extrapolation (using the MELANIE7-II software) to the two nearest landmarks.

Matching with Primary Master Image

Images were edited to remove gross artefacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

Cross-Matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE7 II 2D PAGE analysis program was used to calculate and record approximately 500-700 matched feature pairs between the primary master and each of the other images. The accuracy of this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the KRFs, 4) the apparent molecular weight (MW) of the KRFs, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

Statistical Analysis of the Profiles

The complementary statistical strategies specified below were used in the order in which they are listed to identify KRFs from the MCIs within the mastergroup.

The Wilcoxon Rank-Sum test. This test was performed between the control and the kidney response samples for each MCI basis. The MCIs which recorded a p-value less than or equal to 0.05 were selected as statistically significant KRFs with 95% selectivity.

A second non-overlapping selection strategy is based on the fold change. A fold change representing the ratio of the average normalized protein abundances of the KRFs within an MCI, was calculated for each MCI between each set of controls and kidney response samples. A 95% confidence limit for the mean of the fold changes was calculated. The MCIs with fold changes which fall outside the confidence limit were selected as KRFs which met the criteria of the significant fold change threshold with 95% selectivity. Because the MCI fold changes are based on a 95% confidence limit, it follows that the significant fold change threshold is itself 95%,

A third non-overlapping selection strategy is based on qualitative presence or absence alone. Using this procedure, a percentage feature presence was calculated across the control samples and kidney response samples for each MCI which was a potential KRF based on such qualitative criteria alone, i.e. presence or absence. The MCIs which recorded a percentage feature presence of 95% or more on kidney response samples and a percentage feature presence of 5% or less on control samples, were selected as the qualitative differential KRFs with 95% selectivity. A second group of qualitative differential KRFs with 95% selectivity were formed by those MCIs which recorded a percentage feature presence of 95% or more on control samples and a percentage feature presence of 5% or less on kidney response samples.

Application of these three analysis strategies allowed KRFs to be selected on the basis of: (a) statistical significance as measured by the Wilcoxon Rank-Sum test, (c) a significant fold change threshold with a chosen selectivity, or (b) qualitative differences with a chosen selectivity.

Recovery and Analysis of Selected Proteins

Proteins in KRFs were robotically excised and processed to generate tryptic digest peptides. Tryptic peptides were analyzed by mass spectrometry using a PerSeptive Biosystems Voyager—DE™ STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U. K.) equipped with a nanoflow™ electrospray Z-spray source. For partial amino acid sequencing and identification of KRPIs uninterpreted tandem mass spectra of tryptic peptides were searched using the SEQUEST search program (Eng et al., 1994, J. Am. Soc. Mass Spectrom. 5:976-989), version v.C.I. Criteria for database identification included: the cleavage specificity of trypsin; the detection of a suite of a, b and y ions in peptides returned from the database, and a mass increment for all Cys residues to account for carbamidomethylation. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art and the method described in PCT Application No. PCT/GB01/04034, which is incorporated herein by reference in its entirety, was also used to interpret mass spectra. (In the case of interpretation of low-energy fragmentation mass spectra of peptide ions see Gaskell et al., 1992, Rapid Commun. Mass Spectrom. 6:658-662)

Results

These initial experiments identified: 180 features that were decreased and 136 features that were increased in tissue from subjects having kidney response as compared with tissue from subjects free from kidney response; 24 features that were decreased and 39 features that were increased in blood from subjects having kidney response as compared with blood from subjects free from kidney response. Details of these KRFs are provided in Tables I, II, III and IV. Partial amino acid sequences were determined for the differentially present KRPIs in these KRFs. Details of these KRPIs are provided in Tables VII, VIII, 1× and X.

Using the reference protocol, proteins in kidney cortex tissue and proteins in blood from animals having kidney response (gentamicin-treated animals) and control subjects were separated by isoelectric focusing followed by 2-D gel electrophoresis and analysed by mass spectrometry as described in Section 6.1.14

TABLE XVI
KRFs Identified in Kidney Cortex Tissue of Subjects
having Gentamicin induced kidney response

| | | | Fold Change | | | | | | | |
|---|---|---|---|---|---|---|---|---|---|---|
| | | | Day 8 | | | | Day 22 | | | |
| | | | 0.1 | 1 | 10 | 40 | 0.1 | 1 | 10 | 40 |
| KRF | pI | MW (Da) | mg/kg/day | | | | mg/kg/day | | | |
| KRF-1 | 5.1 | 43,557 | −8.62 | −1.99 | −1.26 | | | | | |
| KRF-2 | 7.3 | 35,621 | −6.17 | −1.16 | −1.4 | | | | | |
| KRF-3 | 4.9 | 39,951 | −5.52 | −1.41 | | −5.52 | | | | |
| KRF-4 | 5.1 | 101,577 | −4.28 | −1.22 | | −4.28 | | | | |
| KRF-5 | 4.9 | 33,363 | −2.42 | | | −2.37 | | | | |
| KRF-6 | 5.3 | 67,007 | −2.13 | | | −1.75 | | | | |
| KRF-7 | 5.4 | 28,601 | −2.12 | | | −1.76 | | | | |
| KRF-8 | 5 | 24,350 | −2.07 | | | | | −1.66 | −2.39 | 1.6 |
| KRF-9 | 6.5 | 37,386 | −1.98 | | | | | | | |
| KRF-10 | 7.2 | 46,674 | −1.88 | | | −2.25 | | | | |
| KRF-11 | 5.4 | 41,863 | −1.73 | −1.54 | −1.7 | | | | | |
| KRF-12 | 5.1 | 63,105 | −1.7 | | −1.2 | −1.57 | | | | |
| KRF-13 | 5.4 | 21,765 | −1.69 | | | −1.8 | | | | |
| KRF-14 | 6.8 | 12,639 | −1.63 | | | −2.95 | | | | |
| KRF-15 | 5 | 25,902 | −1.59 | | | −1.5 | | | | |
| KRF-16 | 5.2 | 21,913 | −1.58 | | | −1.51 | | | | |
| KRF-17 | 5.9 | 33,673 | −1.55 | | | −6.72 | | | | |
| KRF-18 | 5.2 | 81,710 | −1.52 | −1.06 | −1.06 | | | | | |
| KRF-19 | 7 | 21,399 | −1.51 | | | −1.45 | | | | |
| KRF-20 | 6.1 | 26,255 | −1.48 | | | | | | | −1.31 |
| KRF-21 | 5.4 | 80,627 | −1.44 | | | −1.75 | | | | |
| KRF-22 | 5.2 | 39,194 | −1.4 | −1.65 | 1.15 | | | | | |
| KRF-23 | 7.2 | 20,698 | −1.29 | −1.35 | −1.61 | −1.74 | | | | |
| KRF-24 | 8 | 23,594 | −1.29 | | | | | | | |
| KRF-25 | 5.3 | 20,828 | −1.22 | | | −1.6 | | | | |
| KRF-26 | 7.8 | 31,756 | −1.21 | −1.07 | | −6.85 | | | | |
| KRF-27 | 4.9 | 31,623 | −1.13 | | | 1.14 | | | | |
| KRF-28 | 5.6 | 42,298 | 1.16 | −1.86 | | | | | | 1.7 |
| KRF-29 | 5.6 | 38,745 | | −1.86 | −2.01 | −2.28 | | | | |
| KRF-30 | 5.5 | 17,155 | | −1.82 | | 1.83 | 1.58 | | | |
| KRF-31 | 5.1 | 65,723 | | −1.77 | | −1.56 | | | | |
| KRF-32 | 5.7 | 18,083 | | −1.75 | | −1.5 | | | | |
| KRF-33 | 5.2 | 18,968 | | −1.7 | | −1.76 | | | | |
| KRF-34 | 5.6 | 35,836 | | −1.7 | | −1.82 | | | | |
| KRF-35 | 5.7 | 34,167 | | −1.42 | | −1.43 | | | | |
| KRF-36 | 5.6 | 58,058 | | −1.38 | | 1.11 | | | | |
| KRF-37 | 4.7 | 14,017 | | −1.28 | | −1.29 | | | | |
| KRF-38 | 5.2 | 16,833 | | −1.23 | | 1.18 | | | | |
| KRF-39 | 5.7 | 25,316 | | −1.21 | −1.15 | −1.12 | | | | |
| KRF-40 | 5.3 | 80,900 | | −1.03 | | −1.31 | | | | |
| KRF-41 | 5.8 | 43,502 | | | −2.25 | −2.39 | | | | |
| KRF-42 | 5.8 | 39,836 | | | −1.93 | −2.18 | | | | |
| KRF-43 | 6.8 | 21,939 | | | −1.91 | −10.96 | | | | |
| KRF-44 | 5.3 | 41,834 | | | −1.85 | −1.57 | | | | |
| KRF-45 | 7.1 | 23,849 | | | −1.82 | −2.09 | | −2.52 | | |
| KRF-46 | 7.3 | 23,602 | | | −1.79 | −2.17 | | | | |
| KRF-47 | 6.1 | 37,336 | | | −1.78 | | | | | 1.15 |
| KRF-48 | 5 | 59,778 | | | −1.73 | −1.48 | | | | |
| KRF-49 | 6.1 | 42,207 | | | −1.53 | −1.69 | | | | |
| KRF-50 | 7.7 | 49,647 | | | −1.5 | −1.37 | | | | |
| KRF-51 | 6.9 | 34,872 | | | −1.49 | 1.29 | | | | |
| KRF-52 | 7.1 | 14,187 | | | −1.48 | −1.98 | | | | |
| KRF-53 | 6.7 | 28,930 | | | −1.43 | −1.51 | | | | |
| KRF-54 | 7.7 | 26,100 | 1.06 | 1.64 | −1.35 | −1.24 | | | | |
| KRF-55 | 5 | 18,626 | | | −1.33 | −1.4 | | | | |
| KRF-56 | 6 | 43,514 | | | −1.3 | | | | | −1.31 |
| KRF-57 | 6.8 | 11,462 | | | −1.28 | −1.57 | | | | |
| KRF-58 | 5.9 | 80,299 | | | −1.26 | −1.25 | | | | |
| KRF-59 | 5.7 | 27,218 | | | −1.25 | −1.75 | | | | −1.12 |
| KRF-60 | 5.3 | 20,135 | | | −1.24 | −1.49 | | | −1.15 | |
| KRF-61 | 4.7 | 12,754 | | | −1.22 | −6.92 | | | | |
| KRF-62 | 6 | 22,665 | | | −1.17 | −1.76 | | | | −3.47 |
| KRF-63 | 6.4 | 32,486 | | | −1.12 | −9.19 | | | | |
| KRF-64 | 6.5 | 38,483 | | | | −1.31 | | | | |
| KRF-65 | 5.9 | 38,705 | | | | −8.94 | | | | |
| KRF-66 | 6.9 | 22,363 | | | | −7.46 | | | | |
| KRF-67 | 7.6 | 45,480 | | 1.3 | | −5.82 | | | | |
| KRF-68 | 6.1 | 49,829 | 1.05 | | | −5.78 | | | | |
| KRF-69 | 7.4 | 21,692 | | | | −2.54 | | | | |
| KRF-70 | 7.7 | 20,347 | | | | −2.5 | | | | |
| KRF-71 | 6.5 | 23,591 | | | | −2.3 | | | | |
| KRF-72 | 7.6 | 37,026 | | | | −2.21 | | | | |
| KRF-73 | 7.3 | 27,831 | | | | −2.18 | | | | |
| KRF-74 | 5 | 11,914 | | | | −2.15 | | | | |
| KRF-75 | 5.3 | 59,546 | | | | −2.13 | | | | |
| KRF-76 | 7 | 24,556 | | | | −2.12 | | | | |
| KRF-77 | 6.2 | 53,362 | | | | −2.08 | | | | |
| KRF-78 | 8.3 | 33,363 | | | | −2.06 | | | | |
| KRF-79 | 5.7 | 22,899 | | | | −2.06 | | | | |
| KRF-80 | 6.7 | 13,087 | | | | −2.01 | | | | |
| KRF-81 | 5.2 | 64,776 | | | | −1.92 | | | | |
| KRF-82 | 5.7 | 43,557 | | | | −1.92 | −1.24 | | | |
| KRF-83 | 7.1 | 20,828 | | | | −1.9 | | | | |
| KRF-84 | 6.3 | 21,397 | | | | −1.85 | | | | |
| KRF-85 | 7.3 | 18,969 | | | | −1.84 | | | | |
| KRF-86 | 5.6 | 11,175 | | | | −1.81 | | | | |
| KRF-87 | 6 | 62,820 | | | | −1.8 | | | | |
| KRF-88 | 7.7 | 18,953 | | | | −1.8 | | | | |
| KRF-89 | 6.5 | 21,473 | | | | −1.79 | | | | |
| KRF-90 | 8.5 | 16,508 | | | | −1.78 | | | | |
| KRF-91 | 6 | 13,898 | | | | −1.76 | | | | |
| KRF-92 | 5 | 58,397 | | | | −1.74 | | | | |
| KRF-93 | 5.8 | 38,705 | | | | −1.73 | | | | |
| KRF-94 | 5.3 | 16,190 | | | | −1.73 | | | | |
| KRF-95 | 6.2 | 70,946 | | | | −1.7 | | | | |
| KRF-96 | 8 | 27,637 | | | | −1.69 | | | | |
| KRF-97 | 5.4 | 12,570 | | | | −1.68 | | | | 2.01 |
| KRF-98 | 6.1 | 20,618 | | | | −1.67 | | | | |
| KRF-99 | 5.2 | 36,031 | | | | −1.66 | | | | |
| KRF-100 | 7.6 | 24,966 | | | | −1.66 | | | | |
| KRF-101 | 7.7 | 24,269 | | | | −1.66 | | | | |
| KRF-102 | 5.6 | 25,071 | | | | −1.65 | | | | |
| KRF-103 | 5.9 | 45,139 | | | | −1.64 | | | | |
| KRF-104 | 7.1 | 26,948 | | | | −1.64 | | | | |
| KRF-105 | 9.4 | 34,066 | | | | −1.63 | | | | |
| KRF-106 | 7.5 | 31,908 | | | | −1.62 | | | | |
| KRF-107 | 7.1 | 12,919 | | | | −1.61 | | | | |
| KRF-108 | 7.9 | 12,011 | | | | −1.61 | | | | |
| KRF-109 | 6.1 | 55,825 | | | | −1.59 | | | | |
| KRF-110 | 6.8 | 20,454 | | | | −1.53 | | | | |
| KRF-111 | 5.8 | 18,533 | 1.39 | | | −1.5 | | | | |
| KRF-112 | 5.9 | 36,106 | | | | −1.5 | | | | 1.29 |
| KRF-113 | 7.1 | 35,304 | | | | −1.49 | | | | |
| KRF-114 | 5 | 19,067 | | | | −1.48 | | | | |
| KRF-115 | 7.7 | 40,678 | | | | −1.47 | | | | |
| KRF-116 | 6.9 | 34,066 | | | | −1.46 | | 1.1 | | |
| KRF-117 | 6.8 | 10,596 | | | | −1.46 | | | | |
| KRF-118 | 6.1 | 37,985 | | | | −1.45 | | | | |
| KRF-119 | 7.6 | 17,845 | | | | −1.45 | | | | |
| KRF-120 | 5.7 | 40,982 | | | | −1.44 | | | | −1.23 |
| KRF-121 | 4.8 | 46,728 | | | | −1.41 | | | | |
| KRF-122 | 5.3 | 11,763 | | | | −1.4 | | | | |
| KRF-123 | 5 | 44,701 | | | | −1.38 | | | | |
| KRF-124 | 5.6 | 33,463 | | | | −1.38 | | | | |
| KRF-125 | 7.2 | 22,363 | | | | −1.36 | | | | |
| KRF-126 | 5.2 | 64,776 | | | | −1.34 | | | | |
| KRF-127 | 4.6 | 38,483 | | | | −1.32 | | | | |
| KRF-128 | 7.5 | 28,930 | | | | −1.3 | | | | |
| KRF-129 | 6.3 | 19,571 | | | | −1.3 | | | | |
| KRF-130 | 7.3 | 23,929 | | | | −1.29 | | | | |
| KRF-131 | 7.9 | 37,143 | | | | −1.28 | | | | |
| KRF-132 | 7 | 36,051 | | | | −1.18 | | | | |
| KRF-133 | 4.6 | 27,322 | | | | −1.18 | | | | |
| KRF-134 | 5.6 | 24,011 | | | | −1.13 | | | | |
| KRF-135 | 5.2 | 31,880 | | | | −1.06 | | | | |
| KRF-136 | 4.5 | 13,709 | | | | −1.06 | | | | |
| KRF-137 | 6.4 | 40,102 | | | | −1.04 | | | | |
| KRF-138 | 7.6 | 35,652 | | | | | −1.71 | −1.81 | −2.64 | |
| KRF-139 | 7.1 | 27,742 | | | | | −1.21 | −1.42 | −1.34 | |
| KRF-140 | 7.1 | 34,055 | 1.44 | | | 2 | −1.2 | | | |
| KRF-141 | 8.6 | 33,255 | | | | | −1.17 | | | |
| KRF-141 | 8.6 | 33,255 | | | | 1.22 | | | | |
| KRF-142 | 6 | 78,163 | | | | 1.69 | −1.16 | | | −1.54 |
| KRF-143 | 7.7 | 26,909 | | | | | | −1.82 | −1.36 | −1.89 |
| KRF-144 | 6.8 | 23,369 | | | | 1.52 | | −1.53 | | |
| KRF-145 | 7.2 | 22,977 | | | | 1.19 | | −1.5 | −1.28 | |
| KRF-146 | 7.9 | 30,881 | | | | | | −1.31 | | −2.76 |
| KRF-147 | 5.8 | 25,350 | | | | | | −1.08 | | 1.13 |
| KRF-148 | 6.2 | 51,783 | | | | | | −1.02 | | 1.66 |
| KRF-149 | 7.4 | 51,414 | | | | 1.72 | | | −1.63 | −1.5 |
| KRF-150 | 7.4 | 39,580 | | | | 1.6 | | | −1.52 | |
| KRF-151 | 6.5 | 59,042 | | | | | | | −1.39 | 1.03 |
| KRF-152 | 5.2 | 57,842 | | | | | | | | −6.19 |
| KRF-153 | 5.7 | 55,401 | | | | | | | | −2.77 |
| KRF-154 | 8 | 41,346 | | | | | | | | −2.21 |
| KRF-155 | 5.4 | 75,406 | | | | | | | | −2.09 |
| KRF-156 | 5.3 | 27,323 | | | | | | | | −1.98 |
| KRF-157 | 7.5 | 27,600 | | | | | | | | −1.81 |
| KRF-158 | 5.5 | 67,349 | | | | 1.53 | | | | −1.78 |
| KRF-159 | 6.9 | 40,414 | | | | | | | | −1.78 |
| KRF-160 | 5.1 | 34,378 | | | | | | | | −1.74 |
| KRF-161 | 7.9 | 48,455 | | | | | | | | −1.69 |
| KRF-162 | 6.9 | 54,354 | | | | 1.7 | | | | −1.68 |
| KRF-163 | 6 | 79,341 | | | | | | | | −1.58 |
| KRF-164 | 5.9 | 36,047 | | | | | | | | −1.45 |
| KRF-165 | 6.3 | 23,223 | | | | | | | | −1.45 |
| KRF-166 | 6 | 55,886 | | | | | | | | −1.4 |
| KRF-167 | 5 | 38,259 | | | | | | | | −1.39 |
| KRF-168 | 8.9 | 24,933 | | | | | | | | −1.37 |
| KRF-169 | 5.5 | 17,857 | | | | | | | | −1.37 |
| KRF-170 | 8.8 | 26,806 | | | | | | | | −1.33 |
| KRF-171 | 5.5 | 48,755 | | | | | | | | −1.32 |
| KRF-172 | 5.6 | 38,758 | | | | | | | | −1.32 |
| KRF-173 | 8.3 | 20,702 | | | | | | | | −1.3 |
| KRF-174 | 5.8 | 56,049 | | | | | | | | −1.28 |
| KRF-175 | 6 | 72,833 | | | | | | | | −1.23 |
| KRF-176 | 6.9 | 53,667 | | | | | | | | −1.23 |
| KRF-177 | 5.2 | 60,527 | | | | | | | | −1.22 |
| KRF-178 | 6.6 | 22,591 | | | | | | | | −1.2 |
| KRF-179 | 8.7 | 27,848 | | | | | | | | −1.18 |
| KRF-180 | 5.5 | 57,804 | | | | | | | | −1.07 |
| KRF-181 | 8.1 | 19,167 | | | | | | 1.47 | | |
| KRF-182 | 5.6 | 49,449 | 1.2 | | | 1.26 | | | | |
| KRF-183 | 7.9 | 34,066 | 1.28 | | 1.37 | 1.46 | | | | |
| KRF-184 | 6.2 | 45,875 | 1.42 | | | 1.48 | | | | |
| KRF-185 | 5.7 | 44,444 | 1.44 | | 1.32 | 1.56 | | | | |
| KRF-186 | 6.2 | 35,095 | 1.59 | 1.39 | | 1.6 | | | | |
| KRF-187 | 6.3 | 23,924 | 1.61 | | | 1.75 | | | | |
| KRF-188 | 6.3 | 42,667 | 1.63 | 1.5 | | 1.81 | | | | |
| KRF-189 | 7.5 | 37,358 | 1.72 | | | 1.96 | | | | |
| KRF-190 | 4.9 | 35,233 | 1.76 | | | 1.41 | | | | |
| KRF-191 | 6.4 | 56,575 | 2.06 | 1.83 | 1.85 | 2.6 | | | | |
| KRF-192 | 6.8 | 22,439 | | | 1.35 | 1.67 | | | | 1.32 |
| KRF-193 | 5.9 | 94,481 | | | | 1.04 | | | | |
| KRF-194 | 7 | 27,848 | | | | 1.16 | | | | |
| KRF-195 | 6.9 | 35,471 | | | | 1.21 | | | | |
| KRF-196 | 4.7 | 26,603 | | | | 1.25 | | | | |
| KRF-197 | 6 | 24,011 | | | | 1.26 | | | | |
| KRF-198 | 6.8 | 70,766 | | | | 1.27 | | | | |
| KRF-199 | 6.1 | 50,793 | | | | 1.28 | | | | |
| KRF-200 | 6.1 | 31,963 | | | | 1.29 | | | | |
| KRF-201 | 6 | 46,540 | | | | 1.3 | | | | |
| KRF-202 | 5.5 | 31,104 | | | | 1.32 | | | | |
| KRF-203 | 7.5 | 30,601 | | | | 1.32 | | | | |
| KRF-204 | 5.2 | 40,414 | | | | 1.33 | | | | |
| KRF-205 | 7.1 | 81,188 | | | | 1.36 | | | | |
| KRF-206 | 7.6 | 54,603 | | | | 1.38 | | | | |
| KRF-207 | 7.5 | 81,314 | | | | 1.39 | | | | |
| KRF-208 | 4.8 | 15,906 | | | | 1.41 | | | | |
| KRF-209 | 5.7 | 95,301 | | | | 1.42 | | | | |
| KRF-210 | 8 | 35,549 | | | | 1.43 | | | | |
| KRF-211 | 6.3 | 64,776 | | | | 1.44 | | | | |
| KRF-212 | 5.7 | 67,595 | | | | 1.46 | | | | |
| KRF-213 | 8 | 30,983 | | | | 1.46 | | | | |
| KRF-214 | 6.1 | 51,951 | | | | 1.47 | | | | |
| KRF-215 | 8.2 | 27,487 | | | | 1.47 | | | | |
| KRF-216 | 5.6 | 54,508 | | | | 1.48 | | | | |
| KRF-217 | 5.7 | 64,234 | | | | 1.49 | | | | |
| KRF-218 | 5.9 | 48,123 | | | | 1.49 | | | | |
| KRF-219 | 7.4 | 13,463 | | | | 1.49 | | | | |
| KRF-220 | 6.5 | 12,044 | | | | 1.51 | | | | |
| KRF-221 | 7.7 | 57,174 | | | | 1.52 | | 1.04 | | |
| KRF-222 | 7.5 | 57,015 | | | | 1.52 | | | | |
| KRF-223 | 6.7 | 48,914 | | | | 1.52 | | | | |
| KRF-224 | 7.7 | 48,686 | | | | 1.54 | | | | |
| KRF-225 | 6 | 50,369 | | | | 1.56 | | | | |
| KRF-226 | 6.2 | 49,593 | | | | 1.57 | | | | |
| KRF-227 | 7.5 | 60,995 | | | | 1.58 | | | | |
| KRF-228 | 6.3 | 46,688 | | | | 1.59 | | | | |
| KRF-229 | 7.5 | 22,173 | | | | 1.59 | | | | |
| KRF-230 | 9 | 29,375 | | | | 1.6 | | | | |
| KRF-231 | 5.8 | 53,501 | | | | 1.61 | | 1.31 | | |
| KRF-232 | 7.1 | 40,809 | | | | 1.62 | | | | |
| KRF-233 | 5.5 | 68,054 | | | | 1.63 | | | | |
| KRF-234 | 4.9 | 18,919 | | | | 1.63 | | | | |
| KRF-235 | 7.1 | 43,682 | | | | 1.65 | | | | |
| KRF-236 | 5.5 | 13,445 | | | | 1.66 | | | | |
| KRF-237 | 9.1 | 23,172 | | | | 1.67 | | | | |
| KRF-238 | 7.6 | 60,624 | | | | 1.75 | | | | |
| KRF-239 | 7.8 | 59,197 | | | | 1.77 | | | | |
| KRF-240 | 7.5 | 22,637 | | | | 1.78 | | | | |
| KRF-241 | 5.3 | 73,537 | | | | 1.8 | | | | |
| KRF-242 | 7.6 | 69,306 | | | | 1.8 | | | | |
| KRF-243 | 5.5 | 34,330 | | | | 1.8 | | | | |
| KRF-244 | 6.8 | 63,473 | | | | 1.94 | | | | |
| KRF-245 | 4.7 | 43,086 | | | | 1.94 | | | | |
| KRF-246 | 6.3 | 35,903 | | | | 1.95 | | | | |
| KRF-247 | 7.3 | 59,544 | | | | 1.96 | | | | |
| KRF-248 | 4.8 | 18,268 | | | | 1.96 | | | | |
| KRF-249 | 5.4 | 70,401 | | | | 1.98 | | | | |
| KRF-250 | 7.6 | 59,990 | | | | 2.02 | | | | |
| KRF-251 | 7 | 53,029 | | | | 2.03 | | | | |
| KRF-252 | 4.9 | 53,963 | | | | 2.11 | | | | |
| KRF-253 | 9.6 | 48,151 | | | | 2.18 | | | | |
| KRF-254 | 6.7 | 87,067 | | | | 2.19 | | | | |
| KRF-255 | 4.8 | 12,818 | | | | 2.23 | | | | |
| KRF-256 | 5.3 | 13,604 | | | | 2.69 | | | | |
| KRF-257 | 4.7 | 12,867 | | | | 2.93 | | | | |
| KRF-258 | 5.9 | 16,238 | | | | 3.36 | | | | |
| KRF-259 | 5.6 | 86,368 | | | | 3.5 | | | | |
| KRF-260 | 5.5 | 58,378 | | | | 4.42 | | | | |
| KRF-261 | 5.4 | 47,412 | | | | 4.88 | | | | |
| KRF-262 | 7.8 | 23,749 | | | | 5.08 | | | | |
| KRF-263 | 7.7 | 42,563 | | | | 5.7 | | | | |
| KRF-264 | 5.4 | 31,429 | | | | | | 1.38 | | 1.05 |
| KRF-265 | 6.1 | 43,075 | | | | | | 2.02 | | 2.28 |
| KRF-266 | 5.5 | 23,258 | | | | | 2.49 | 2.35 | | 1.41 |
| KRF-267 | 5.6 | 28,492 | | | | | | | 1.11 | 1.08 |
| KRF-268 | 5.7 | 21,058 | | | | | | | | 1.03 |
| KRF-269 | 6 | 38,864 | | | | | | | | 1.05 |
| KRF-270 | 6.7 | 47,112 | | | | | | | | 1.08 |
| KRF-271 | 6.9 | 30,062 | | | | | | | | 1.1 |
| KRF-272 | 6.1 | 40,034 | | | | | | | | 1.12 |
| KRF-273 | 4.7 | 31,342 | | | | | | | | 1.2 |
| KRF-274 | 5.6 | 27,218 | | | | | | | | 1.2 |
| KRF-275 | 4.9 | 21,618 | | | | | | | | 1.2 |
| KRF-276 | 6.5 | 60,624 | | | | | | | | 1.22 |
| KRF-277 | 4.6 | 37,808 | | | | | | | | 1.29 |
| KRF-278 | 7.2 | 78,547 | | | | | | | | 1.31 |
| KRF-279 | 5.8 | 46,599 | | | | | | | | 1.33 |
| KRF-280 | 6.5 | 43,914 | | | | | | | | 1.36 |
| KRF-281 | 4.9 | 30,750 | | | | | | | | 1.36 |
| KRF-282 | 4.7 | 15,768 | | | | | | | | 1.38 |
| KRF-283 | 5 | 28,061 | | | | | | | | 1.4 |
| KRF-284 | 6 | 26,976 | | | | | 1.75 | | | 1.43 |
| KRF-285 | 5.8 | 46,740 | | | | | | | | 1.43 |
| KRF-286 | 5.6 | 22,363 | | | | | | | | 1.43 |
| KRF-287 | 5.5 | 36,325 | | | | | | | | 1.51 |
| KRF-288 | 5.1 | 40,583 | | | | | | | | 1.69 |
| KRF-289 | 5.5 | 20,307 | | | | | | | | 2 |

TABLE XVII
KRFs Identified in Blood of Subjects having
Gentamicin induced kidney response

| | | MW | Fold Change |
|---|---|---|---|
| KRF | pI | (Da) | 40 mg/kg/day |
| KRF-290 | 5.3 | 124,107 | −2.56 |
| KRF-291 | 8.7 | 69,580 | −1.87 |
| KRF-292 | 7.3 | 81,357 | −1.72 |
| KRF-293 | 5.6 | 136,203 | −1.65 |
| KRF-294 | 5.7 | 135,486 | −1.6 |
| KRF-295 | 5.7 | 123,856 | −1.56 |
| KRF-296 | 5.3 | 99,803 | −1.56 |
| KRF-297 | 5.3 | 23,260 | −1.5 |
| KRF-298 | 7 | 87,673 | −1.49 |
| KRF-299 | 4.8 | 52,986 | −1.47 |
| KRF-300 | 6.1 | 134,812 | −1.43 |
| KRF-301 | 4.9 | 52,180 | −1.38 |
| KRF-302 | 4.8 | 53,467 | −1.35 |
| KRF-303 | 5 | 77,747 | −1.34 |
| KRF-304 | 6.9 | 53,475 | −1.33 |
| KRF-305 | 7.2 | 50,919 | −1.31 |
| KRF-306 | 4.8 | 78,125 | −1.29 |
| KRF-307 | 6.3 | 136,964 | −1.24 |
| KRF-308 | 4.8 | 59,584 | −1.24 |
| KRF-309 | 6.8 | 49,184 | −1.24 |
| KRF-310 | 5.6 | 95,157 | −1.23 |
| KRF-311 | 5.3 | 114,923 | −1.22 |
| KRF-312 | 5.7 | 17,513 | −1.2 |
| KRF-313 | 4.9 | 53,018 | −1.16 |
| KRF-314 | 5.7 | 35,921 | 1.13 |
| KRF-315 | 6.2 | 88,662 | 1.15 |
| KRF-316 | 5.4 | 65,170 | 1.15 |
| KRF-317 | 6.3 | 87,681 | 1.17 |
| KRF-318 | 5.6 | 33,267 | 1.17 |
| KRF-319 | 4.7 | 33,621 | 1.18 |
| KRF-320 | 6.1 | 89,623 | 1.19 |
| KRF-321 | 6 | 58,883 | 1.2 |
| KRF-322 | 5.9 | 70,153 | 1.21 |
| KRF-323 | 5.9 | 32,933 | 1.22 |
| KRF-324 | 6.1 | 56,989 | 1.24 |
| KRF-325 | 5.4 | 24,595 | 1.24 |
| KRF-326 | 5.6 | 15,368 | 1.27 |
| KRF-327 | 5.9 | 47,074 | 1.28 |
| KRF-328 | 5.9 | 22,165 | 1.28 |
| KRF-329 | 5.7 | 100,420 | 1.3 |
| KRF-330 | 5.1 | 79,642 | 1.3 |
| KRF-331 | 7.1 | 47,142 | 1.3 |
| KRF-332 | 5.9 | 66,491 | 1.31 |
| KRF-333 | 5.8 | 67,137 | 1.32 |
| KRF-334 | 4.4 | 12,184 | 1.32 |
| KRF-335 | 5.9 | 95,725 | 1.33 |
| KRF-336 | 5.9 | 23,420 | 1.33 |
| KRF-337 | 5.8 | 97,397 | 1.39 |
| KRF-338 | 5.8 | 71,160 | 1.39 |
| KRF-339 | 6.4 | 44,084 | 1.39 |
| KRF-340 | 6 | 51,612 | 1.4 |
| KRF-341 | 5.8 | 48,456 | 1.4 |
| KRF-342 | 6.1 | 24,316 | 1.42 |
| KRF-343 | 7.8 | 46,948 | 1.44 |
| KRF-344 | 5.8 | 24,239 | 1.44 |
| KRF-345 | 5.6 | 91,497 | 1.49 |
| KRF-346 | 5.8 | 58,085 | 1.52 |
| KRF-347 | 4.6 | 67,652 | 1.57 |
| KRF-348 | 4.8 | 115,177 | 1.68 |
| KRF-349 | 5.3 | 49,677 | 1.69 |
| KRF-350 | 8.3 | 63,976 | 1.83 |
| KRF-351 | 8.5 | 49,211 | 4.79 |
| KRF-352 | 7.8 | 66,706 | 10.8 |

The present invention is not to be limited in terms of the particular embodiments described in this application; which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

When a reference is made herein to a method of treating or preventing a disease or condition using a particular agent or combination of agents, it is to be understood that such a reference is intended to include the use of that agent or combination of agents in the preparation of a medicament for the treatment or prevention of the disease or condition.

Preferred features of each aspect of the invention are as for each of the other aspects mutatis mutandis.

1. A method for screening, diagnosis or prognosis of kidney response in a subject, for determining the stage or severity of kidney response in a subject, for identifying a subject at risk of developing kidney response, or for monitoring the effect of therapy administered to a subject having kidney response, said method comprising:
(a) analyzing a test sample of tissue or body fluid from the subject by two dimensional electrophoresis to generate a two-dimensional array of features, said array comprising one or more Kidney Response-Associated Features (KRF)s selected from the group consisting of KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-105, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288, KRF-289, KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313, KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351 and KRF-352, whose relative abundance correlates with the presence, absence, stage or severity of kidney response or predicts the onset or course of kidney response; and
(b) comparing the abundance of each selected feature in the test sample with the abundance of that chosen feature in tissue or body fluid from one or more subjects free from kidney response, or with a previously determined reference range for that feature in subjects free from kidney response, or with the abundance at least one Expression Reference Feature (ERF) in the test sample.

2. The method according to claim 1, wherein said method is for determining the ability of drug candidates to induce an unwanted kidney response.

3. The method of claim 1, wherein the tissue is kidney tissue and the KRFs are selected from the group consisting of KRF-1, KRF-2, KRF-3, KRF-4, KRF-5, KRF-6, KRF-7, KRF-8, KRF-9, KRF-10, KRF-11, KRF-12, KRF-13, KRF-14, KRF-15, KRF-16, KRF-17, KRF-18, KRF-19, KRF-20, KRF-21, KRF-22, KRF-23, KRF-24, KRF-25, KRF-26, KRF-27, KRF-28, KRF-29, KRF-30, KRF-31, KRF-32, KRF-33, KRF-34, KRF-35, KRF-36, KRF-37, KRF-38, KRF-39, KRF-40, KRF-41, KRF-42, KRF-43, KRF-44, KRF-45, KRF-46, KRF-47, KRF-48, KRF-49, KRF-50, KRF-51, KRF-52, KRF-53, KRF-54, KRF-55, KRF-56, KRF-57, KRF-58, KRF-59, KRF-60, KRF-61, KRF-62, KRF-63, KRF-64, KRF-65, KRF-66, KRF-67, KRF-68, KRF-69, KRF-70, KRF-71, KRF-72, KRF-73, KRF-74, KRF-75, KRF-76, KRF-77, KRF-78, KRF-79, KRF-80, KRF-81, KRF-82, KRF-83, KRF-84, KRF-85, KRF-86, KRF-87, KRF-88, KRF-89, KRF-90, KRF-91, KRF-92, KRF-93, KRF-94, KRF-95, KRF-96, KRF-97, KRF-98, KRF-99, KRF-100, KRF-101, KRF-102, KRF-103, KRF-104, KRF-105, KRF-106, KRF-107, KRF-108, KRF-109, KRF-110, KRF-111, KRF-112, KRF-113, KRF-114, KRF-115, KRF-116, KRF-117, KRF-118, KRF-119, KRF-120, KRF-121, KRF-122, KRF-123, KRF-124, KRF-125, KRF-126, KRF-127, KRF-128, KRF-129, KRF-130, KRF-131, KRF-132, KRF-133, KRF-134, KRF-135, KRF-136, KRF-137, KRF-138, KRF-139, KRF-140, KRF-141, KRF-142, KRF-143, KRF-144, KRF-145, KRF-146, KRF-147, KRF-148, KRF-149, KRF-150, KRF-151, KRF-152, KRF-153, KRF-154, KRF-155, KRF-156, KRF-157, KRF-158, KRF-159, KRF-160, KRF-161, KRF-162, KRF-163, KRF-164, KRF-165, KRF-166, KRF-167, KRF-168, KRF-169, KRF-170, KRF-171, KRF-172, KRF-173, KRF-174, KRF-175, KRF-176, KRF-177, KRF-178, KRF-179, KRF-180, KRF-181, KRF-182, KRF-183, KRF-184, KRF-185, KRF-186, KRF-187, KRF-188, KRF-189, KRF-190, KRF-191, KRF-192, KRF-193, KRF-194, KRF-195, KRF-196, KRF-197, KRF-198, KRF-199, KRF-200, KRF-201, KRF-202, KRF-203, KRF-204, KRF-205, KRF-206, KRF-207, KRF-208, KRF-209, KRF-210, KRF-211, KRF-212, KRF-213, KRF-214, KRF-215, KRF-216, KRF-217, KRF-218, KRF-219, KRF-220, KRF-221, KRF-222, KRF-223, KRF-224, KRF-225, KRF-226, KRF-227, KRF-228, KRF-229, KRF-230, KRF-231, KRF-232, KRF-233, KRF-234, KRF-235, KRF-236, KRF-237, KRF-238, KRF-239, KRF-240, KRF-241, KRF-242, KRF-243, KRF-244, KRF-245, KRF-246, KRF-247, KRF-248, KRF-249, KRF-250, KRF-251, KRF-252, KRF-253, KRF-254, KRF-255, KRF-256, KRF-257, KRF-258, KRF-259, KRF-260, KRF-261, KRF-262, KRF-263, KRF-264, KRF-265, KRF-266, KRF-267, KRF-268, KRF-269, KRF-270, KRF-271, KRF-272, KRF-273, KRF-274, KRF-275, KRF-276, KRF-277, KRF-278, KRF-279, KRF-280, KRF-281, KRF-282, KRF-283, KRF-284, KRF-285, KRF-286, KRF-287, KRF-288 and KRF-289.

4. The method according to claim 3, wherein said method is for determining the ability of drug candidates to induce an unwanted kidney response.

5. The method of claim 1, wherein the body fluid is blood or serum or plasma and the KRFs are selected from the group consisting of KRF-290, KRF-291, KRF-292, KRF-293, KRF-294, KRF-295, KRF-296, KRF-297, KRF-298, KRF-299, KRF-300, KRF-301, KRF-302, KRF-303, KRF-304, KRF-305, KRF-306, KRF-307, KRF-308, KRF-309, KRF-310, KRF-311, KRF-312, KRF-313, KRF-314, KRF-315, KRF-316, KRF-317, KRF-318, KRF-319, KRF-320, KRF-321, KRF-322, KRF-323, KRF-324, KRF-325, KRF-326, KRF-327, KRF-328, KRF-329, KRF-330, KRF-331, KRF-332, KRF-333, KRF-334, KRF-335, KRF-336, KRF-337, KRF-338, KRF-339, KRF-340, KRF-341, KRF-342, KRF-343, KRF-344, KRF-345, KRF-346, KRF-347, KRF-348, KRF-349, KRF-350, KRF-351 and KRF-352.

6. The method according to claim 5, wherein said method is for determining the ability of drug candidates to induce an unwanted kidney response.

7. A method for screening, diagnosis or prognosis of kidney response in a subject, for determining the stage or severity of kidney response in a subject, for identifying a subject at risk of developing kidney response, or for monitoring the effect of therapy administered to a subject having kidney response, said method comprising:
(a) quantitatively detecting, in a sample of kidney tissue from the subject, at least one Kidney Response-Associated Protein Isoform (KRPI) selected from the group consisting of: KRPI-2, KRPI-8, KRPI-11, KRPI-13, KRPI-14, KRPI-15, KRPI-16, KRPI-19, KRPI-21, KRPI-23, KRPI-27, KRPI-28, KRPI-35, KRPI-40, KRPI-41, KRPI-42, KRPI-43, KRPI-45.1, KRPI-45.2, KRPI-57, KRPI-59, KRPI-60, KRPI-63, KRPI-70, KRPI-72, KRPI-73, KRPI-76, KRPI-84, KRPI-85, KRPI-86, KRPI-88, KRPI-90, KRPI-91, KRPI-98, KRPI-101, KRPI-104, KRPI-105, KRPI-113, KRPI-122, KRPI-123, KRPI-128, KRPI-131, KRPI-132, KRPI-134, KRPI-138, KRPI-139, KRPI-142, KRPI-143, KRPI-144, KRPI-149, KRPI-152, KRPI-153, KRPI-158, KRPI-159, KRPI-168, KRPI-170, KRPI-178, KRPI-179, KRPI-183, KRPI-184, KRPI-185, KRPI-186, KRPI-188, KRPI-189.1, KRPI-189.2, KRPI-192, KRPI-196, KRPI-202, KRPI-206, KRPI-208, KRPI-210, KRPI-219, KRPI-222, KRPI-229, KRPI-232, KRPI-235.1, KRPI-235.2, KRPI-236, KRPI-237, KRPI-240, KRPI-245, KRPI-247, KRPI-249, KRPI-250, KRPI-252, KRPI-253, KRPI-256, KRPI-257, KRPI-263, KRPI-267, KRPI-273, KRPI-278, KRPI-280, KRPI-282, KRPI-285 and KRPI-286, and
(b) comparing the level or amount of said isoform or isoforms detected in step (a) with a control.

8. The method according to claim 7, wherein the step of quantitatively detecting comprises testing at least one aliquot of the sample, said step of testing comprising: contacting the aliquot with an antibody that is immunospecific for a preselected KRPI;
(a) quantitatively measuring any binding that has occurred between the antibody and at least one species in the aliquot; and
(b) comparing the results of step (b) to a control.

9. The method according to claim 7, wherein said method is for determining the ability of drug candidates to induce an unwanted kidney response.

10. The method according to claim 7, wherein the subject is a rat.

11. A method for screening, diagnosis or prognosis of kidney response in a subject, for determining the stage or severity of kidney response in a subject, for identifying a subject at risk of developing kidney response, or for monitoring the effect of therapy administered to a subject having kidney response, said method comprising:
(a) quantitatively detecting, in a sample of blood, serum or plasma from the subject, at least one Kidney Response-Associated Protein Isoform (KRPI) selected from the group consisiting of: KRPI-313, KRPI-314.1, KRPI-314.2, KRPI-327.1, KRPI-327.2 and KRPI-339, and
(b) comparing the level or amount of said isoform or isoforms detected in step (a) with a control.

12. The method according to claim 11, wherein the step of quantitatively detecting comprises testing at least one aliquot of the sample, said step of testing comprising:
(a) contacting the aliquot with an antibody that is immunospecific for a preselected KRPI;
(b) quantitatively measuring any binding that has occurred between the antibody and at least one species in the aliquot; and
(c) comparing the results of step (b) to a control.

13. The method according to claim 11, wherein said method is for determining the ability of drug candidates to induce an unwanted kidney response.

14. The method according to claim 11, wherein the subject is a rat.

15. A method for screening, diagnosis or prognosis of kidney response in a subject, for determining the stage or severity of kidney response in a subject, for identifying a subject at risk of developing kidney response, or for monitoring the effect of therapy administered to a subject having kidney response, said method comprising:
(a) contacting at least one oligonucleotide probe comprising 10 or more consecutive nucleotides complementary to a nucleotide sequence encoding a KRPI selected from the group consisting of KRPI-2, KRPI-8, KRPI-11, KRPI-13, KRPI-14, KRPI-15, KRPI-16, KRPI-19, KRPI-21, KRPI-23, KRPI-27, KRPI-28, KRPI-35, KRPI-40, KRPI-41, KRPI-42, KRPI-43, KRPI-45.1, KRPI-45.2, KRPI-57, KRPI-59, KRPI-60, KRPI-63, KRPI-70, KRPI-72, KRPI-73, KRPI-76, KRPI-84, KRPI-85, KRPI-86, KRPI-88, KRPI-90, KRPI-91, KRPI-98, KRPI-101, KRPI-104, KRPI-105, KRPI-113, KRPI-122, KRPI-123, KRPI-128, KRPI-131, KRPI-132, KRPI-134, KRPI-138, KRPI-139, KRPI-142, KRPI-143, KRPI-144, KRPI-149, KRPI-152, KRPI-153, KRPI-158, KRPI-159, KRPI-168, KRPI-170, KRPI-178, KRPI-179, KRPI-183, KRPI-184, KRPI-185, KRPI-186, KRPI-188, KRPI-189.1, KRPI-189.2, KRPI-192, KRPI-196, KRPI-202, KRPI-206, KRPI-208, KRPI-210, KRPI-219, KRPI-222, KRPI-229, KRPI-232, KRPI-235.1, KRPI-235.2, KRPI-236, KRPI-237, KRPI-240, KRPI-245, KRPI-247, KRPI-249, KRPI-250, KRPI-252, KRPI-253, KRPI-256, KRPI-257, KRPI-263, KRPI-267, KRPI-273, KRPI-278, KRPI-280, KRPI-282, KRPI-285, KRPI-286, KRPI-313, KRPI-314.1, KRPI-314.2, KRPI-327.1, KRPI-327.2 and KRPI-339 and orthologs thereof, with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

16. The method of claim 15, wherein step (a) includes the step of hybridizing the nucleotide sequence to a DNA array, wherein one or more members of the array are the probes complementary to a plurality of nucleotide sequences encoding distinct KRPIs.

17. The method according to claim 15, wherein said method is for determining the ability of drug candidates to induce an unwanted kidney response.

18. A diagnostic kit adapted for use in the method of claim 7 comprising a capture reagent capable of capturing a KRPI may additionally optionally comprise one or more of the following:
(1) instructions for using the capture reagent;
(2) a labeled binding partner to the capture reagent;
(3) a solid phase upon which the capture reagent is immobilized; and
(4) a label or insert indicating regulatory approval for use, or any combination thereof.

19. A diagnostic kit adapted for use in the method of claim 11 comprising a capture reagent capable of capturing a KRPI may additionally optionally comprise one or more of the following:
(1) instructions for using the capture reagent;
(2) a labeled binding partner to the capture reagent;
(3) a solid phase upon which the capture reagent is immobilized; and
(4) a label or insert indicating regulatory approval for use, or any combination thereof.
